SEQUENCE LISTING (3 sequences)

SEQ ID NO: 1 - DNA, 19 bp, artificial sequence
  right primer for CYP24
gcagctcgactggagtgac

SEQ ID NO: 2 - DNA, 21 bp, artificial sequence
  left primer for CYP24
catcatggccatcaaaacaat

SEQ ID NO: 3 - DNA, 27 bp, artificial sequence
  oligonucleotide sequence from involucrin
gcgggaggcagatctggcagatactga

(19) United States
(12) Patent Application Publication
(10) Pub. No.: US 2011/0118228 A1
(43) Pub. Date: May 19, 2011

(54) Enzymatic production or chemical synthesis and uses for 5,7-dienes and UVB conversion products thereof

(76) Inventors: Andrzej Slominski, Memphis, TN (US); Wei Li, Germantown, TN (US); Blazej Zbytek, Decatur, GA (US); Robert C. Tuckey, Nedlands (AU); Jordan K. Zjawiony, Oxford, MS (US); Minh Ngoc Nguyen, Morley (AU); Zorica Janjetovic, Memphis, TN (US); Michal A. Zmijewski, Gdansk (PL); Trevor W. Sweatman, Memphis, TN (US); Duane D. Miller, Memphis, TN (US); Jianjun Chen, Memphis, TN (US); Arnold E. Postlethwaite, Eads, TN (US)

(21) Appl. No.: 12/807,178

(22) Filed: Aug. 30, 2010

Related U.S. Application Data
(63) Continuation-in-part of application No. PCT/US2009/001324, filed on Mar. 2, 2009.
(60) Provisional application No. 61/189,798, filed on Aug. 22, 2008.
(60) Provisional application No. 61/067,461, filed on Feb. 28, 2008.

(51) Int. Cl. A61K 31/57; C07J 5/00; C12N 5/00; A61P 35/00; A61P 35/02; A61P 17/00; A61P 19/02; A61P 3/10; A61P 3/04; A61P 9/10; C12P 33/06

(52) U.S. Cl. 514/182; 552/592; 435/375; 435/58

(57) ABSTRACT

Provided herein are steroidal compounds that are androsta-5,7-dienes or a pregna-5,7-dienes and ultraviolet B (UVB) conversion products thereof which includes pharmaceutical compositions of the steroidal compounds as shown in Tables 1 and 2. Also provided is a method for producing hydroxylated metabolites of cholecalciferol or ergocalciferol via the P450scc (CYP11A1) or CYP27B1 enzyme systems where the hydroxylase has an activity to hydroxylate position C20 of a secosteroid or its 5,7-dieneal precursor and the hydroxylated metabolites so produced. In addition, a method for inhibiting proliferation of either a normally or abnormally proliferating cell by contacting the cell with any of the compounds described herein. A related method is provided of treating a condition associated with the proliferating cell such as a cancer, a skin disorder, a defect in cell differentiation, cosmetic, prophylaxsis, or healthy cell maintenance.

### CROSS-REFERENCE TO RELATED APPLICATIONS

This continuation-in-part application claims benefit of priority under 35 U.S.C. §120 of pending international application PCT/US2009/001324, filed Mar. 2, 2009, which claims benefit of priority under 35 U.S.C. §119(e) of provisional U.S. Ser. No. 61/189,798, filed Aug. 22, 2008, and provisional U.S. Ser. No. 61/067,461, filed Feb. 28, 2008, now abandoned, the entirety of all of which are hereby incorporated by reference.

### FEDERAL FUNDING LEGEND

This invention was produced in part using funds obtained through grant R01 AR052190 from the National Institutes of Health. Consequently, the federal government has certain rights in this invention.

### BACKGROUND OF THE INVENTION

1. Field of the Invention

The present invention relates to the fields of steroid chemistry and medicine. More specifically, the present invention relates to the chemical or enzymatic production and therapeutic use of androsta- and pregna-5,7-dienes and their secosteroidal, tachysterol-like and lumisterol-like UVB conversion products.

2. Description of the Related Art

The UVB driven photolysis of the steroidal B ring of cholesta-5,7-dien-3β-ol (7-dehydrocholesterol, 7DHC) w ith further rearrangement of the activated molecule (pre-D3) generates vitamin D3 ((5Z,7E)-9,10-secocholesta-5,7,10(19)-trien-3β-ol, cholecalciferol, D3), tachysterol (6E)-9,10-secocholesta-5(10),6,8-trien-3β-ol, T3) and luminosterol (9β,10α-colesta-5,7-dien-3β-ol, L3) (1-3). Vitamin D3 (D3), the main product of the process plays a fundamental role in biology, serving as a precursor for the hormone 1,25-dihydroxyvitamin D3 ((5Z,7E)-9,10-secocholesta-5,7,10(19)-triene-1α,3β,25-triol, 1,25(OH)₂D3, calcitiol) with its most fundamental role in the regulation of body calcium homeostasis (2, 4-5).

Conversion of 7DHC was demonstrated by the Holick group as a two-step process. The first and rapid step is photolysis of the unsaturated B ring of 7DHC and formation of pre-D3 product. After irradiation, pre-D3 undergoes slow isomerization to three main products: D3, T3 and L3. T3 has shifted double bonds when compared with D3, and L3 is formed by recyclization of the B ring, with reversed configuration at C-9 and C-10. The process of isomerization is accelerated by increased temperature; product formation depends on the absorbed energy and the UVB wavelength.

Recent studies have revealed that mammalian cytochrome P450scc (CYP11A1), in addition to its role in the conversion of cholesterol to pregnenolone for steroid synthesis, can also metabolize vitamins D2 and D3, as well as their provitamin precursors ergosterol and 7-dehydrocholesterol (cholesta-5,7-dien-3β-ol, 7DHC) (6-10). P450scc converts vitamin D3 to 20-hydroxycholecalciferol ((5Z,7E)-9,10-secocholesta-5,7,10(19)-triene-3β,20-diol, 20(OH)D3) and di- and tri-hydroxycholecalciferol in a sequential and stereospecific, manner with initial formation of 20(S)-hydroxycholecalciferol (9). 20-hydroxycholecalciferol is the major product of the reaction indicating that it can be released from the active site of the enzyme with only a minor portion remaining or rebinding for further hydroxylation. It is also the only product of vitamin D3 hydroxylation detected in incubations of isolated adrenal mitochondria. Thus in organs expressing high levels of P450scc such as the adrenal cortex, corpus luteum, follicles and placenta, production of 20-hydroxycholecalciferol could possibly have systemic effects, while in organs expressing low levels of P450scc such as skin (10), it could serve local para-, auto- or intracrine roles.

In humans, after entering the circulation, vitamin D3 can be hydroxylated in the liver to 25-hydroxycholecalciferol ((5Z,7E)-9,10-secocholesta-5,7,10(19)-trien-3β,25-diol, 25(OH)D3) by mitochondrial CYP27A1 (11). On the cellular level, 1,25(OH)₂D3 binds to specific vitamin D receptors (VDR) that heterodimerize with the retinoid X receptors (RXR). Complexes receptor-vitamin affect expression of genes that have vitamin D response elements (VDRE) in their promoter (12). 1α,25-dihydroxycholecalciferol is also synthesized locally by epidermal keratinocytes which contain both 25-hydroxylase and CYP27B1 (13-16). The 1α-hydroxylase activity required to convert 25-hydroxycholecalciferol to calcitriol has also been detected in many other peripheral tissues (14, 16). CYP24 hydroxylates 1α,25-dihydroxycholecalciferol as well as 25-hydroxycholecalciferol to yield metabolically inactive products in the kidney or in a plethora of peripheral tissues (17-18). 1α,25-dihydroxycholecalciferol stimulates CYP24 gene expression and inhibits expression of both CYP27B1 and CYP27A1 genes (11,13,15,17).

While the biological role of 20-hydroxycholecalciferol is unknown, it is well documented that, in addition to its fundamental role in calcium metabolism, 1α,25-dihydroxycholecalciferol and its derivatives have immune and neuroendocrine activities, and tumorostatic and anticarcinogenic properties, affecting proliferation, differentiation and apoptosis in cells of different lineages, and protecting DNA against oxidative damage (19-21). 1α,25-dihydroxycholecalciferol and its derivatives also have significant local actions on formation and functional differentiation of adnexal structures and the epidermis, modulation of skin immune system and protection against UVB-induced DNA damage (2,19,20,22,23).

However, the use of vitamin D3 or its hydroxylated derivatives in treatment of cancer or hyperproliferative disorders is limited, because of hypercalcemic toxicity when used at pharmacological concentrations. Interestingly, the calcemic effect can be strongly reduced by shortening of the side chain (24-25). Also, significantly, there is a paucity of information on the photolytic transformation of steroidal 5,7-dienes to the corresponding D-, L- or T-like compounds.

Thus, there is a need in the art for improved secosteroidal, tachysterol-like and lumisterol-like compounds that are useful as therapeutics. Specifically, the prior art is deficient in androsta- and pregna-5,7-dienes and their UVB irradiation products and there use as therapeutic compounds for cancer and other pathological conditions. The present invention fulfills this long- standing need and desire in the art.

### SUMMARY OF THE INVENTION

The present invention is directed to a steroidal compound that is an androsta-5,7-diene or a pregna-5,7-diene or an ultraviolet B (UVB) conversion product thereof or pharmaceutical compositions thereof. The present invention is directed to a related steroidal compound that is further derivatized with an ester or an ether substituent.

The present invention also is directed to a method for inhibiting proliferation of a cell. The method comprises contacting the cell in vitro or in vivo with one or more compounds identified in one or both of Tables 1 or 2. The present invention is directed to a related method wherein the cell is contacted with a Table 1 or 2 compound(s) derivatized with an ester or ether moiety.

The present invention is directed further to a method for producing one or more hydroxylated metabolites of (5Z,7E)-9,10-secocholesta-5,7,10(19)-trien-3b-ol (cholecalciferol) or (5Z,7E,22E)-9,10-secoergosta-5,7,9(10),22-tetraene-3β-ol (ergocalciferol). The method comprises hydroxylating a substrate of one or both of a cytochrome P450scc (CYP11A1) or CYP27B1 enzyme system in at least one position where the substrate is enzymatically convertible to the hydroxylated cholecalciferol metabolites. The hydroxylase comprises a plant or animal hydroxylase having an activity that hydroxylates position C20 of secosteroid or its 5,7-dieneal precursor.

The present invention is directed further still to a hydroxylated cholecalciferol or ergocalciferol derivative or analog compound that is (5Z,7E)-9,10-secocholesta-5,7,10(19)-triene-3β,20-diol, (5Z,7E)-9,10-secocholesta-5,7,10(19)-triene-3β,20,23-triol, (5Z,7E)-9,10-secocholesta-5,7,10(19)-triene-3β,17α,20,23-tetrol, (5Z,7E)-9, 10-secochalesta-5,7,10(19-triene-1α,3β,20,23-tetrol, or (5Z,7E)-9,10-secocholesta-5,7,10(19)-triene-1α,3β,17α,20,23-pentol (6E)-9,10-secocholesta-5(10),6,8-triene-3β,20α-diol, (6E)-9,10-secocholesta-5(10),6,8-triene-3β,20β-diol, 9β,10α-cholesta-5,7-diene-3β,20α-diol, 9β,10α-cholesta-5,7-diene-3β,20β-diol, cholesta-5,7-diene-3β,20α-diol, cholesta-5,7-diene-β,20β-diol, (5Z,7E,22E)-9,10-secoergosta-5,7,9(10),22-tetraene-3β,20α-diol, (5Z,7E,22E)-9,10-secoergosta-5,7,9(10),22-tetraene-β,20β-diol, (6E,22E)-9,10-secoergosta-5(10),6,8,22-tetraene-3β,20α-diol, 9,10-secoergosta-5(10),6,8,22-tetraene-3β,20α-diol, 9β,10α-ergosta-5,7,22-triene-3β,20α-diol, 9β,10α-ergosta-5,7,22-triene-β,20β-diol, ergosta-5,7,22-triene-3β,20α-diol, or ergosta-5,7,22-triene-β,20β-diol.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 depicts the chemical synthesis of androsta- and pregna-5,7-dienes. Reagents and conditions: (a) Ac₂O, microwave, p-toluenesulfonic acid monohydrate; (b) dibromantin, 2,2′-azobisisobutyronitrile, benzene/hexane (1:1), 100° C., reflux; (c) Bu₄NBr, Bu₄NF, THF, room temperature; (d) LiAlH₄, THF, 0° C.; (e) K₂CO₃,MeOH-THF, room temperature.

FIG. 2 depicts the chemical synthesis of epimers 20R (20R)-9β,10α-pregna-5,7-dien-3b,17a,20-triol; 7L) and 20S (20S)-9β,10α-pregna-5,7-diene-3β,17α,20-triol; 6L) Reagents and conditions: (a) Ac₂O, microwave, p-toluenesulfonic acid monohydrate; (b) dibromantin, 2,2′-azobisisobutyronitrile, benzene/hexane (1:1), 100° C., reflux; (c) Bu₄NBr, Bu₄NF, THF, room temperature; (d) LiAlH₄, THF, 0° C.

FIGS. 3A-3C depict chemical syntheses (FIG. 3A), the retention times (FIG. 3B) and UV absorption spectra (FIG. 3C) of (5Z,7E)-9,10-secopregna-5,7,10(19)-triene-3β,20-diol and (5Z,7E)-9,10-secocholesta-5,7,9(10)-triene-3β,20-diol and their tachysterol-like and lumisterol-like analogs. FIG. 3A reagents and conditions: (a) dibromantin, 2,2′azobisisobutyronitrile, benzene/hexane (1:1), 100° C.; (b) Bu₄NBr, Bu₄NF, THF, room temperature; (c) Mg, THF, 45° C.; (d) THF, 0° C-RT. FIGS. 3D-3E depict the natural enzymatic synthesis of 9β,10α-cholesta-5,7-diene-3β,20-diol, or 20(OH)-ergosterol, via the P450scc enzyme system (FIG. 3D) and the chemical synthesis (FIG. 3E) of 20(OH)-ergosterol and its (5Z,7E,22E)-9,10-secoergosta-5,7,9(10),22-tetraene-3β,20-diol, tachysterol-like a lumisterol-like analogs. FIG. 3D conditions: (a) UVB photolysis of ergosterol, (b) P450scc and hydrolysis. FIG. 3E reagents and conditions: (a) Dibromantin, 2,2′-azobisisobutyronitrile, benzene/hexane (1:1), 100° C., reflux; (b) Bu₄NBr, Bu₄NF, THF, room temperature; (c) CrCl₂, CH₃; (d) Mg, THF, 0° C.; (e) THF, −78° C.; (f) UVB photolysis, g. HPLC separation.

FIG. 4 depicts the chemical synthesis of vitamin D3-like 17-carboxy-acid (5Z,7E)-3hydroxy-9,10-secoandrosta-5,7,9(10)-triene-17β-carboxylic acid) and its tachysterol-like and lumisterol-like analogs. Reagents and conditions: (a) Ac₂O, microwave, p-toluenesulfonic acid monohydrate; (b) Dibromantin, 2,2′-azobisisobutyronitrile, benzene/hexane (1:1), 100° C., reflux; (c) Bu₄NBr, Bu₄NF, THF, room temperature; (d) K₂CO₃, MeOH-THF, room temperature, overnight.

FIG. 5 depicts the photolysis of androsta- and pregna-5,7-dienes.

FIGS. 6A-6E illustrate the dynamics of UVB-driven photolysis of 3β-hydroxypregna-5,7-dien-20-one (5c). In FIG. 6A compound 5c was irradiated for 20 minutes (top chromatogram) or 60 minutes (others). Samples were incubated in the dark, at room temperature (20° C.) and analyzed by RP-HPLC 1, 24 and 96 hours after irradiation. Chromatograms were recorded at 280 nm. FIG. 6B depicts representative UV spectra of 5c and products of its irradiation. FIG. 6C illustrates that UVB dose (time of irradiation) dependent conversion of 5c monitored by relative quantification of substrate to products. Equal amounts of 5c were irradiated for 0, 2, 5, 15, 30 and 60 minutes, incubated for 24 hours at room temperature and analyzed by RP-HPLC. FIGS. 6D-6E depict temperature dependent isomerization of 5c irradiation products. The relative changes in amount of substrate and products after irradiation for 15 minutes followed by incubation for various time (as shown) at 20° C. (FIG. 6D) or 37° C. (FIG. 6E). The results in FIGS. 6C-6E were expressed as a percentage of total area under the selected peak (at 280 nm) to the total area of all peaks at 280 nm.

FIGS. 7A-7D depicts proton NMR spectra of androsta- and pregna-5,7-dienes and main products of their irradiation with 5a as an example; 3β,17α-dihydroxypregna-5,7-dien-20-one (5a) (FIG. 7A), 3β,17α-dihydroxy-9,10-pregna-5,7-dien-20-one (5a-L) (FIG. 7B), (5Z,7E)-3β,17α-dihydroxy-9,10-secopregna-5,7,10(19)-trien-20-one (5a-D) (FIG. 7C), a (6E)-3β,17α-dihydroxy-9,10-secopregna-5(10),6,8-trien-20-one (5a-T) (FIG. 7D). The main peaks used for structural identification of compounds are marked by number of carbon. Impurities and solvents are described or marked with a star (*).

FIG. 8 depicts the photolysis of pregna-5,7-diene-3β,17α,20-triols.

FIGS. 9A-9B depict the UVB-driven photolysis of 4S FIG. 9A shows the RP-HPLC separation of 4S irradiation products after treatment with UVB for 15 minutes. FIG. 9B is the representative UV spectra of irradiated samples. Peak number (FIG. 9A) corresponds to predicted structure based on UV spectra (FIG. 9B). Peaks were assigned as follows: 1-4, 10, 11—isoT-like and oxidized isoT-like; 5—5S; 6—4S-L; 7—4S-D; 8—4S; 9—4S-T; 12—4S-pD. The UV spectra for RP-HPLC (FIG. 8A) were measured at 280 nm.

FIGS. 10A-10H illustrates the dose (time of irradiation) dependence of pregna-5,7-diene-3β,17α,20-triols (4R and 4S) photo-conversion. The compound 4R (FIGS. 10A, 10C, 10E, and 10G) or 4S (FIGS. 10B, 10D, 10F, and 10H) was irradiated for 5 min (FIGS. 10A-10B), 15 min (FIGS. 10C-10D), 30 min (FIGS. 10E-10F) or 60 min (FIGS. 10G-10H) min and products were separated by using RP-HPLC. The absorption profiles of products were recorded by using diode-array detector. The samples were separated after incubation for 2 hours at room temperature.

FIGS. 11A-11B show the identity of oxidized derivatives of 4S (FIG. 11A) and 4R (FIG. 11B) by mass spectrometry. Sample were purified after UV treatment using RP-HPLC and analyzed with LC-MS.

FIGS. 12A-12D are chromatograms showing products of and time course for vitamin D3 metabolism by P450scc. Vitamin D3 (50 μM) dissolved in cyclodextrin to a final concentration of 0.45%, was incubated with 1.0 μM P450scc for 1 h in a reconstituted system containing adrenodoxin and adrenodoxin reductase. Samples were extracted and analyzed by reverse-phase HPLC. FIG. 12A is the test reaction. FIG. 12B shows the control incubation (zero time) showing the vitamin D3 substrate and the methanol gradient used for elution. RT, retention time in min. Abbreviations for products are: monohydroxyvitamin D3, (OH)D3; dihydroxyvitamin D3, (OH)2D3; trihydroxyvitamin D3, (OH)3D3. The time course for metabolism of vitamin D3 in cyclodextrin showing consumption of cholecalciferol and major metabolites (FIG. 12C) and minor metabolites (FIG. 12D). Vitamin D3 (50 AA) dissolved in cyclodextrin to a final concentration of 0.45% was incubated with 1.0 μM P450scc for the indicated times.

FIGS. 13A-13D demonstrate hydroxylation of 20-hydroxycholecalciferol and 20,23-dihydroxycholecalciferol by P450scc. FIG. 13A is the time-course for metabolism of 20-hydroxycholecalciferol (50 μM) dissolved in 0.45% cyclodextrin and incubated with 1.0 μM P450scc. HPLC chromatograms showing metabolism of 20,23-dihydroxycholecalciferol by P450scc from a 1 h incubation in 0.45% cyclodextrin in a test reaction (FIG. 13B), zero-time control where the reaction mixture was extracted at the end of the pre-incubation (FIG. 13C) and in a test reaction spiked with 1 nmol standard trihydroxyvitamin D3 purified as for the WR experiments (FIG. 13D).

FIGS. 14A-14E depict proton NMR spectra and expansion of proton-carbon HSQC of vitamin D3, dihydroxyvitamin D3 and trihydroxyvitamin D3. The NMR spectra are cholecalciferol (FIG. 14A), dihydroxy metabolite identified as 20,23-dihydroxycholecalciferol (FIG. 14B) and trihydroxy metabolite identified as 17α,20,23-trihydroxycholecalciferol. The peaks marked by * are from unidentified impurities. Expansion of proton-carbon HSQC of the two metabolites for 3-CH and 23-CH are shown in FIGS. 14C-14D, respectively. Abbreviations are as for FIGS. 11A-11D.

FIGS. 15A-15C depict the identification of 20,23-dihydroxycholecalciferol. Expansion of proton-proton COSY correlations for 3-CH and 23-CH (FIG. 15A), expansion of proton-proton TOCSY correlations for 3-CH and 23-CH (FIG. 15B) and expansion of proton-carbon HSQC showing groups having correlation to 3-CH and 23-CH (FIG. 15C) are shown.

FIGS. 16A-16C depict the identification of 17α,20,23-trihydroxycholecalciferol. Expansion of proton-carbon HSQC of 20,23-dihydroxycholecalciferol showing the three methine groups (FIG. 16A), expansion of the same region of 17α,20,23-trihydroxycholecalciferol. 25-CH is intact where 17-CH is missing and 14-CH is shifted (FIG. 16B) and expansion of proton-proton COSY showing the correlation from 14-CH to 15-CH₂, and from 15-CH₂ to 16-CH₂ (FIG. 16C), are shown. Abbreviations are as for FIGS. 1A-1D.

FIGS. 17A-17C show products of and the time course for 1α-dihydroxycholecalciferol metabolism by P450scc. In FIG. 17A 1α-dihydroxycholecalciferol (50 μM) dissolved in cyclodextrin to a final concentration of 0.45%, was incubated with 1.0 μM P450scc for 1 h in a reconstituted system containing adrenodoxin and adrenodoxin reductase. Samples were extracted and analyzed by reverse-phase HPLC. FIG. 17B is a control incubation (zero time) showing the 1α-hydroxycholecalciferol substrate. Abbreviations for products are: monohydroxyvitamin D3, (OH)D3; dihydroxyvitamin D3, (OH)₂D3; trihydroxyvitamin D3 (OH)₃D3. RT, retention time in min. FIG. 17C is a time course for metabolism of 1α-hydroxycholecalciferol by P450scc in cyclodextrin. 1α-hydroxycholecalciferol (50 μM) dissolved in cyclodextrin to a final concentration of 0.45%, was incubated with 2.0 μM P450scc.

FIG. 18 depicts pathways for metabolism of vitamin D3 by P450scc. The major pathway is highlighted with bolded arrows.

FIGS. 19A-19D demonstrate that 20-hydroxycholecaciferol inhibits keratinocyte proliferation. HaCaT keratinocytes were treated for 48 hours in DMEM containing 5% charcoal-treated FBS. [³H]-thymidine was added for last 12 hours of incubation and then DNA synthesis was assessed (FIG. 19A). HaCaT keratinocytes were cultured for 10 days in DMEM containing 5% charcoal-treated FBS. Then colonies were fixed, stained with crystal violet and counted. Representative pictures of HaCaT treated with 20(OH)D3 at 10⁻⁸ M or controls are shown (FIG. 19B). Graphs showing the dose-dependent effect (FIG. 19C) and comparison to the effects of other secosteroids at 10⁻⁸ M (FIG. 19D) are presented. Data are presented as mean±SEM ([³H]-thymidine assay: n=36; colony forming assay: n=4), *P<0.05 **P<0.005, ***P<0.0005.

FIGS. 20A-20C demonstrates that 20-hydroxycholecalciferol stimulates expression of involucrin mRNA in normal human epidermal keratinocytes. Keratinocytes were incubated in EpiLife medium containing EDGS supplement with either 20-hydroxycholecalciferol, 25-hydroxycholecalciferol or 1α,25-dihydroxycholecalciferol, then lysed and total RNA extracted and reverse transcribed. Involucrin mRNA levels were measured with reagent Hs00846307_s1 according to the manufacturer's protocol (Applied Biosystems, Foster City, Calif.) and normalized to 18SrRNA content. Data are presented as mean±SEM (n=3). *P<0.05 versus control, **P<0.005 versus control, ***P<0.0005 versus control, #p<0.05 versus 1α,25-dihydroxycholecalciferol. FIG. 20A is the time response at 10⁻⁸ M. FIG. 20B is the dose response at 6 hours. FIG. 20C is a comparison of vitamin D3 hydroxy-derivatives at 10⁻⁸ M and 6 hours.

FIGS. 21A-21B demonstrate that 20-hydroxycholecalciferol stimulates expression of involucrin in keratinocytes. In FIG. 21A HaCaT keratinocytes were incubated for 48 hours in DMEM medium containing 5% FBS with 20-hydroxycholecalciferol or vehicle and then fixed and stained with involucrin antibody followed by secondary antibody linked to FTC. Cells were then read with a flow cytometer as described previously. Data are presented as mean±SEM (n=4). Black: isotype control, blue: cells treated with vehicle only, red: cells treated with 20-hydroxycholecalciferol. In FIG. 21B normal epidermal keratinocytes treated with 20-hydroxycholecalciferol or vehicle and then stained with anti-involucrin antibody followed by secondary antibody linked to RTC. Cells were photographed as described in Example 1. Magnification: 20×.

FIGS. 22A-22C demonstrate that 20-hydroxycholecalciferol inhibits expression of CYP27B1 mRNA. Normal epidermal keratinocytes were incubated in EpiLife medium containing EDGS supplement with either 20-hydroxycholecalciferol, 25-hydroxycholecalciferol or 1α,25-dihydroxycholecalciferol, then lysed and total RNA extracted and reverse transcribed. CYP27B1 mRNA levels were measured with reagent Hs00168017_m1 according to manufacturer's protocol (Applied Biosystems, Foster City, Calif.) and normalized to 18SrRNA content as described. Data are presented as mean±SEM (n=3). *P<0.05 versus control, **P<0.005 versus control, ***P<0.0005 versus control. FIG. 22A is the time response at 10⁻⁸ M. FIG. 22B is the dose response at 1 h. FIG. 22C is the comparison of relative potencies at 10⁻⁸ M and 1 hour.

FIGS. 23A-23B demonstrate that 20-hydroxycholecalciferol stimulates expression of CYP24 mRNA in keratinocytes. Normal epidermal keratinocytes were incubated in EpiLife medium containing EDGS supplement with 20-hydroxycholecalciferol, then lysed and total RNA extracted and reverse transcribed. CYP24 mRNA levels were measured as described in Methods section. Data are presented as mean±SEM (n=3). *P<0.05 versus control, **P<0.005 versus control, ***P<0.0005 versus control. FIG. 23A is the time response at 10⁻⁶ M. FIG. 23B is the dose response (at 24 h).

FIGS. 24A-24D demonstrate that 20-hydroxycholecalciferol stimulates VDRE through VDR in HaCaT keratinocytes. In FIG. 24A cells were stimulated for 24 h with 10 nM 20-hydroxycholecalciferol, then nuclear extracts prepared and incubated with labelled VDRE. Arrows indicate protein-DNA complex that contains RXR. Result representative of three experiments. FIG. 24B shows control cells and cells stimulated with 1α,25-dihydroxycholecalciferol. In FIG. 24C cells were transfected with VDRE-Luc and with scrambled or VDR siRNA and then incubated for 24 h with 10 nM 20(OH)D3 or with vehicle (control). Data are presented as mean±SEM (n=4), **P<0.005, versus untreated control, #P<0.00005 versus scrambled siRNA and treatment with 20-hydroxycholecalciferol. In FIG. 24D cells were transfected with scrambled or VDR siRNA and after 24 h whole cell lysates were prepared and expression of VDR and beta-actin was assessed with Western blot using the same amount of proteins.

FIG. 25A-25D demonstrate that 20-hydroxycholecalciferol induces S arrest and apoptosis in human breast carcinoma cell line MD-MBA-231 (FIG. 25A), in human osteosarcoma cell line MG-63 (FIG. 25B) and in human prostate carcinoma cell line PC-3(FIG. 25C) and induces G1/G0 arrest and apoptosis in human radial growth phase amelanotic melanoma WM35 cells (FIG. 25D). Cells were seeded in 96-well plate and incubated with 20-hydroxycholecalciferol in DMEM medium containing 5% FBS for 48 h (FIGS. 25A) or 72 h (FIG. 25C) or for 24 h after a 12 h synchronization in serum free medium (FIG. 25D). [3^{H}]-thymidine (1 μM/ml) was added for a final 18 h (FIGS. 25A, 25D) or 12 h (FIG. 25C) of incubation or cells were assessed with sulforhodamine assay (FIG. 25C). Data is shown as mean±SEM (n=5; FIGS. 24C) or mean ±SEM (n=6; FIGS. 24A, 24C, 24D) and was analyzed using GraphPrism 4.0.

FIGS. 26A-26B demonstrate suppression of [³H]-thymidine incorporation into DNA by 1α,20-dihydroxycholecalciferol. HaCaT keratinocytes were treated with 1α,20-dihydroxycholecalciferol for 24 h (FIG. 26A) or 48 h (FIG. 26B). Differences to control (ethanol vehicle) and between each dose are significant (p<0.05).

FIG. 27 shows the results of a sulforhodamine B assay for the toxicity of 1α,20-dihydroxycholecalciferol. HaCaT keratinocytes were treated with the indicated concentrations of 1α,20-dihydroxycholecalciferol for 48 h, then stained with sulforohadamine b and the absorbance measured at 565 nm. Differences to control (ethanol vehicle) and between each dose are significant (p<0.05).

FIGS. 28A-28B demonstrates that 1α,20-dihydroxycholecalciferol increases CYP24 mRNA levels in HaCaT keratinocytes. HaCaT keratinocytes were treated with 0.1 μM or 10 μM 1,20(OH)₂D3 for 6 h (FIG. 28A) or 24 h (FIG. 28B). RNA was measured by RT-PCR and is expressed relative to Cyclophylin B as a house keeping gene.

FIGS. 29A-29B demonstrate the dose dependent suppression of [³H]-thymidine incorporation under increasing vitamin D3 concentration: 20,23(OH)₂D3 (FIG. 29A) and 1,25(OH)₂D3 (FIG. 29B). Differences to control (ethanol treated cells) and between each dose are significant (p<0.05).

FIGS. 30A-30D demonstrate dose dependent suppression in colony forming ability under increasing 20,23(OH)₂D3 concentration in AbC1 hamster melanoma cells (FIG. 30A) and SK Mel 188 human melanoma cells (FIG. 30B). Visualization of SK Mel 188 colonies in soft agar formed after treatment with 10 nM ethanol solvent (FIG. 30C) and 10 nM 20,23(OH)₂D3 (FIG. 30D) and stained with MTT reagents. Decreased number and size of colonies has been observed in both cell lines. Differences to control (ethanol treated cells) and between each dose are significant (p<0.05)/

FIG. 31 demonstrates that 20,23-dihydroxycholecalciferol arrests HaCaT cells at G1/0 and G2/M cell cycle phase. HaCaT cells were treated for 24 h with 20,23(OH)₂D3 and 1,25(OH)₂D3 at 10 nM concentration. Then the cells were harvested, fixed, stained with PI and read with flow cytometer. Data is presented as mean±SD (n=3), p<0.05 between control and treatment.

FIG. 32A-32B demonstrate that 20,23-dihydroxycholecalciferol stimulates expression of involucrin in HaCaT cells. In FIG. 32A HaCaT cells were treated for 24 h with 20,23(OH)₂D3 and 1,25(OH)₂D3 at 10 nM concentration. Then the cells were fixed, stained with anti Involucrin (green) antibody and nuclei with PI (red). Cells were checked under the fluorescent microscope using 20× magnification. Noticeable increase in cell size is observed after treatment with 20,23(OH)₂D3 and 1,25(OH)₂D3 compared to control. In FIG. 32B the cells were harvested by trypsinisation, fixed in 2% PFA, stained with anti Involucrin antibody and and read with flow cytometer. Data is presented as mean±SD (n=3), p<0.05 between control and treatment has shown the increase in expression of invoulucrin. IgG is used as a control.

FIGS. 33A-33C demonstrate that 20,23-dihydroxycholecalciferol stimulates expression of Cyp24 and VDRE in HaCaT cells. HaCaT cells were transfected with luciferase constructs: Cyp24 (FIG. 33B), empty vector pLuc and VDRE (FIG. 33C) alone or with human VDR receptor (FIG. 33A) using lipofectamine. Posttransfection cells were treated for 24 h with ethanol as a vehicle, 20,23(OH)₂D3 and 1,25(OH)₂D3 at 10 nM and 100 nM concentration. Than cells were lysed in lysis buffer and luciferase activity measured on luminometer. Data is presented as meantSD (n=4), p<0.05.

FIGS. 34A-34D demonstrate that 20,23-dihydroxycholecalciferol inhibits NFκB-Luc activity in HaCaT and normal human keratinocytes. HaCaT cells (FIGS. 34A-34B) and normal keratinocytes, third passage, (FIGS. 34C-34D) were transfected with luciferase construct NFiB-Luc using lipofectamine. 24 h posttransfection cells were treated for indicated period of time with ethanol as a vehicle, 20,23(OH)₂D3 (FIGS. 34A, 34C) and 1,25(OH)₂D3 (FIGS. 34B, 34D) at 100 nM concentration. The cells were lysed in lysis buffer and luciferase activity measured on luminometer.

FIGS. 35A-35E demonstrate that 20,23-dihydroxycholecalciferol increases NFκBI (IκB-α) protein levels in keratinocytes. HaCaT (FIGS. 35A-35D) and normal keratinocytes (FIG. 35E) were treated with 20,23(OH)₂D3 and 1,25 (OH)₂D3 at the concentration of 100 nM for 30 min, 1 h, 4 h, 16 h and 24 h. Cells were lysed and 25 μg of proteins from whole cell extract was loaded onto gel. Proteins were transferred to PVDF membrane and exposed to primary antibodies: anti-IκB-α(FIGS. 35A, 35D-35E), NFκB-p65 (FIG. 35B) and anti-β actin (FIG. 35C).

FIGS. 36A-36E demonstrate the effect of the 20-hydroxycholecalciferol (FIG. 36A), 20,23-dihydroxycholecalciferol (FIG. 36B), 1α,25-dihdroxycholecalciferol (FIG. 36C), 1α,20-dihydroxycholecalciferol (FIG. 36D) 17α,20,23-trihydroxycholcalciferol (FIG. 36E) on proliferation of HaCaT keratinocytes 48 h after treatment with [³H]-thymidine. *p<0.05; **p<0.01.

FIGS. 37A-37G illustrate that compounds 20-OH pD3 and 20-OH pL3 inhibit proliferation of SKMEL-188 human melanoma cells (FIGS. 37A-37B) and epidermal HaCaT keratinocytes (FIG. 37C), inhibit colony formation on soft agar of AbC1 melanoma cell colonies greater than 0.2 mm (FIGS. 37D-37E) and 0.5 mm (FIGS. 37F-37G), respectively.

FIGS. 38A-38F illustrate that vitamin D3-like compound pD3 inhibits proliferation of epidermal HaCaT keratinocytes (FIG. 38A), colony formation on soft agar of SKMEL-188 human melanoma cells (FIG. 38B) and PC3 human prostate cancer cells (FIG. 38C) and inhibits NFiB in HaCaT cells (FIG. 38D). Compound aD3 inhibits colony formation on soft agar of SKMEL-188 human melanoma cells (FIGS. 38E-38F).

FIGS. 39A-39E illustrate that compounds 17α,20-diOH pL3 (FIGS. 39A-39B) and 17α,20-diOH pD3 (FIGS. 39C-39E) inhibit proliferation of epidermal HaCaT keratinocytes (FIGS. 39A-39D) and melanoma cells (FIG. 39E).

FIG. 40 illustrates that 17-carboxylic acid inhibits DNA synthesis in epidermal HaCaT keratinocytes. Concentrations: 0.01, 0.10, 1.0, 10, and 100 nM 17-COON; data are shown as mean±SEM (n=4). *p<0.05.

FIGS. 41A-41C illustrates that compounds pD3 (PD3), 20-OH pL3 (PL3) and 20-OH pD3 (200H pD3) induce differentiation of K562 human chronic myeloid leukemia cells (FIG. 41A) and inhibits proliferation of K562 cells (FIG. 41B) and mouse erytholeukemia cells (Mel) (FIG. 41C) treated for 7 days at 10⁻⁷M. Negative control is addition of vehicle and positive control is 1α,25(OH)₂D3 (1,25(OH)₂D3).

FIG. 42 illustrates that compounds pD3 (PD3) and 20-OH pL3 (PL3) induce monocytic differentiation in HL-60 and U937 human leukemia cell lines as evidenced by the appearance of monocytic cells (blue) compared to control under the microscope.

FIG. 43 illustrates that 20(OH)D₃ suppresses collagen-induced arthritis (CIA) in female DBA/1 Lac J mice (n=24).

FIGS. 44A-44B illustrate that the vitamin D3 analog 17,20Sdi(OH)pD inhibits Type I collagen production (FIG. 44A) and that the vitamin D3 analogs 17,20Rdi(OH)pD and 17,20Sdi(OH)pD reduce TGF-β1 induced Col1A1 mRNA. T

FIG. 45 illustrates that 20(OH)D₃ prevents bleomycin-induced scleroderma in C57BU6 mice. Total collagen at skin injection site of C567BU6 mice was measured after 21 days of treatment with bleomycin, bleomycin+20(OH)D₃ or vehicle.

FIGS. 46A-46E illustrate that 20(OH)D₂ increased involucrin gene (FIG. 46A) and involucrin protein (FIG. 46B) expression which is demonstrated by increase in cell numbers expressing involucrin (FIG. 46C), total relative fluorescence (FIG. 46D) and fluorescent area (FIG. 46E).

FIGS. 47A-47E illustrate the inhibitory effects of 20(OH)D₂ (FIG. 47A) compared to 1,25(OH)₂D₃ (FIG. 47B). Colonies over 0.2 nm (FIGS. 47A, 47C) and 0.5 nm (FIGS. 47B, 47C) were counted. Colonies in the presence of control, 20(OH)D₂ and 1,25(OH)₂D₃ are shown (FIG. 47E).

FIGS. 48A-48F illustrate DNA synthesis inhibition, as measured by cell proliferative ability after incubation with 20(OH)D₂ and 1,25(OH)₂D₃, in HaCaT keratinocytes for 48 h (FIG. 48A) or 72 h (FIG. 48B), in normal epidermal melanocytes (FIG. 48C), neonatal epidermal melanocytes (FIG. 48D), SKMEL-188 human melanoma cells (FIG. 48E) and AbC1 hamster melanoma cells (FIG. 48F).

### DETAILED DESCRIPTION OF THE INVENTION

As used herein, the term “a” or “an”, when used in conjunction with the term “comprising” in the claims and/or the specification, may refer to “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

As used herein, the term “or” in the claims refers to “and/or” unless explicitly indicated to refer to alternatives only or the alternatives are mutually exclusive, although the disclosure supports a definition that refers to only alternatives and “and/or.”

As used herein, the term “contacting” refers to any suitable method of bringing one or more of the compounds described herein or other inhibitory or stimulatory agent into contact with proliferative cells, or a tissue comprising the same, associated with a pathophysiological condition. In vitro or ex vivo this is achieved by exposing the proliferative cells or tissue to the compound(s) in a suitable medium. For in vivo applications, any known method of administration is suitable as described herein.

As used herein, the terms “effective amount” or “pharmacologically effective amount” are interchangeable and refer to an amount that results in an a delay or prevention of onset of the cell proliferation and/or pathophysiological condition or results in an improvement or remediation of the symptoms of the same. Those of skill in the art understand that the effective amount may improve the patient's or subject's condition, but may not be a complete cure of the disease, disorder and/or condition.

As used herein, the term “inhibit” refers to the ability of the steroidal compounds described herein, to block, partially block, interfere, decrease, reduce or deactivate enzymes associated with the unwanted cell proliferation. As used herein, the term “stimulate” refers to the ability of the steroidal compounds to increase differentiation of keratinocytes. The steroidal compounds described herein are effective as both inhibitor and stimulator compounds.

As used herein, the term “neoplastic cell” or refers to a cell or a mass of cells or tissue comprising the neoplastic cells characterized by, inter alia, abnormal cell proliferation. The abnormal cell proliferation results in growth of these cells that exceeds and is uncoordinated with that of the normal cells and persists in the same excessive manner after the stimuli which evoked the change ceases or is removed. Neoplastic cells or tissues comprising the neoplastic cells show a lack of structural organization and coordination relative to normal tissues or cells which usually results in a mass of tissues or cells which can be either benign or malignant. As would be apparent to one of ordinary skill in the art, the term “tumor” refers to a mass of malignant neoplastic cells or a malignant tissue comprising the same.

As used herein, the term “treating” or the phrase “treating a tumor” or “treating a neoplastic cell” or “treating a neoplasm” includes, but is not limited to, halting the growth of the neoplastic cell or tumor, killing the neoplastic cell or tumor, or reducing the number of neoplastic cells or the size of the tumor. Halting the growth refers to halting any increase in the size or the number of neoplastic cells or tumor or to halting the division of the neoplastic cells. Reducing the size refers to reducing the size of the tumor or the number of or size of the neoplastic cells.

As used herein, particularly in the drawings and the description thereof, the terms “20(OH)D3 or 20-hydroxycholecalciferol”, “25(OH)D3 or 25-hydroxycholecalciferol”, “1,20(OH)₂D3 or 1,20-hydroxycholecalciferol”, “1,25(OH)₂D3 or 1,25-dihydroxycholecalciferol”, “20,23(OH)₂D3 or 20,23-dihydroxycholecalciferol”, “1,20,23(OH)₂D3 or 1,20,23-trihydroxycholecalciferol”, and “17,20,23(OH)₃D3 or 17,20,23-trihydroxycholecalciferol” refer to mono-, di- and tri-hydroxy derivatives of cholecalciferol, i.e., vitamin D3. Also, the terms “20(OH)D2” or “20(OH)D₂” refer to the mono-hydroxy derivative of ergosterol, i.e., vitamin D2. Additional abbreviations that may be used for other androsta-5,7-dienes, pregna-5,7-dienes or ergosta-5,7-dienes and 5,6,8-trienes, including the secosterol, tachysterol-like and lumisterol-like ultraviolet B (UVB) conversion or chemically synthesized products are found in Tables 1 and 2 with the chemical names. Furthermore, if not specifically named to indicate an enantiomer, isomer, chirality, stereochemistry etc., the chemical names of any compound disclosed herein, if applicable, is considered to encompass any possible chemical orientation. In a non-limiting example, 3β,20-diol substituents encompass a 20α- or 20β-diol.

As used herein, the term “subject” refers to any target of the treatment.

In one embodiment of the present invention there is provided steroidal compound that is an androsta-5,7-diene or a pregna-5,7-diene or an ultraviolet B (UVB) conversion product thereof or pharmaceutical compositions thereof. In this embodiment the steroidal compound may be identified in Table 1. Further to this embodiment the Table 1 steroidal compound may be derivatized to comprise another or an ester substituent. Also, in this embodiment the UV conversion product of the steroidal compound may be produced in vivo or in vitro.

In another embodiment of the present invention there is provided a method for inhibiting proliferation of a cell comprising contacting the cell with one or more compounds identified in one or both of Tables 1 or 2. Further to this embodiment the steroidal compounds in Table 1 or Table 2 may be derivatized to comprise an ether or an ester substituent.

In these embodiments the steroidal compounds in Table 1 or Table 2 may be one or more of an androsta-5,7-diene or a pregna-5,7-diene where the compound is converted in vivo to a corresponding ultraviolet B conversion compound after contacting the cell. Also, the cell may be a normally proliferating cell or an abnormally proliferating neoplastic cell. Examples of the cell are an adrenal cell, a gonadal cell, a keratinocyte or melanocyte, a pancreatic cell, a cell from the gastrointestinal tract, a prostate cell, a breast cell, a lung cell, an immune cell, a hematologic cell, a kidney cell, a brain cell, a cell of neural crest origin, a skin cell, a mesenchymal cell, a leukemia cell, a melanoma cell, or an osteosarcoma cells.

In these embodiments the cell may be in vivo and is associated with a pathophysiological condition in a subject. In one aspect the condition is associated with neoplastic cells. Examples of a neoplastic condition are melanoma, squamous cell carcinoma, breast carcinoma, prostate carcinoma, lung carcinoma, sarcoma, carcinoma, lymphoma, leukemia, or brain tumor. In another aspect the condition is cosmetic, prophylaxis or maintenance of healthy proliferating cells.

In yet another aspect of these embodiments the condition may be a skin or mucosal disorder or a defect in cell differentiation. In this aspect the skin disorder may be a hyperproliferative skin disorder, a pigmentary skin disorder, an inflammatory skin disorder, or other skin disorder characterized by hair growth on legs, arms, torso, or face, or alopecia, or skin aging, skin damage or a pre-carcinogenic state. Examples of a hyperprofliferative skin disorder are psoriasis or a keloid or fibromatosis, the pigmentary skin disorder is vitiligo, the inflammatory or autoimmune skin disorder is pemphigus, bullous pemphigoid, allergic contact dermatitis, atopic dermatitis, or lupus erythematosus.

In yet another aspect of these embodiments the condition may be associated with undifferentiated cells or defectively differentiated cells where contact.further induces differentiation thereof. In this aspect the condition may result from an activity of NFκβ directed against proliferating cells or immune cells. Examples of such condition are an autoimmune disease or an inflammatory process associated with NFκβ activity in keratinocytes, immunocompetent cells of the skin, the immune cells of the systemic immune system, or present in autoimmune diseases. Particularly the autoimmune disease or inflammatory process is scleroderma or morphea, keloid or fibromatosis, rheumatoid arthritis, multiple sclerosis, inflammatory bowel diseases, interstitial cystitis, diabetes, obesity atherosclerosis, vasculities, or gout.

In yet another embodiment of the present invention there is provided a method for producing an hydroxylated metabolite of (5Z,7E)-9,10-secocholesta-5,7,10(19)-trien-3β-ol, (cholecalciferol) (5Z,7E,22E)-9,10-secoergosta-5,7,9(10),22-tetraene-3β-ol (ergocalciferol) comprising hydroxylation a substrate of one or both of a cytochrome P450scc (CYP11A1) or CYP27B1 enzyme system in at least one position where the substrate is enzymatically convertible to the hydroxylated cholecalciferol metabolite where the hydroxylase is a plant or animal hydroxylase having an activity that hydroxylates position C20 of secosteroid or its 5,7-dieneal precursor.

In this embodiment the substrate may be cholecalciferol or ergocalciferol or (5Z,7E)-9,10-secocholesta-5,7,10(19)-triene-1α,3β-diol and one or both of C17 or a side chain thereof in the substrates is hydroxylated. In one aspect at least C20 within the C17 side chain may be hydroxylated. In this aspect the enzymatically produced hydroxylated cholecalciferol is (5Z,7E)-9,10-secocholesta-5,7,10(19)-triene-3β,20-diol, (5Z,7E)-9,10-secocholesta-5,7,10(19)-triene-1α,3β,20-triol, (5Z,7E)-9,10-secocholesta-5,7,10(19)-triene-3β,20,23-triol, (5Z,7E)-9,10-secocholesta-5,7,10(19)-triene-1α,3β,20,23-tetrol, (6E)-9,10-secocholesta-5(10),6,8-triene-3β,20α-diol, (6E)-9,10-secocholesta-5(10),6,8-triene-3β,20b-diol, 9β,10α-cholesta-5,70-diene-3β,20α-diol, 9β,10α-cholesta-5,7-diene-3β,20β-diol, cholesta-5,7-diene-3β,20α-diol, cholesta-5,7-diene-β,20β-diol, (5Z,7E,22E)-9,10-secoergosta-5,7,9(10),22-tetraene-3β,20α-diol, (5Z,7E,22E)-9,10-secoergosta-5,7,9(10),22-tetraene-β,20β-diol, (6E,22E)-9,10-secoergosta-5(10),6,8,22-tetraene-3β,20α-diol, (6E,22E)-9,10-secoergosta-5(10),6,8,22-tetraene-3β,20β-diol, 9b,10a-ergosta-5,7,22-triene-3β,20α-diol, 9b,10a-ergosta-5,7,22-triene-β3,20β-diol, ergosta-5,7,22-triene-3β,20α-diol, or ergosta-5,7,22-triene-β,20β-diol. In another aspect C17 and at least C20 within the C17 side chain may be hydroxylated. In this aspect the enzymatically produced hydroxylated cholecalciferol is (5Z,7E)-9,10-secocholesta-5,7,10(19)-triene-3β,17α,20,23-tetrol or (5Z,7E)-9,10-secocholesta-5,7,10(19)-triene-1α,3β,17α,20,23-pentol.

Also in this embodiment the cytochrome P450scc enzyme system may be an in vitro system, comprising cytochrome P450scc enzyme, adrenodoxin, adrenodoxin reductase, and NADPH. In addition, the enzyme system(s) comprises a mammalian cell, a plant cell, an insect cell, a yeast cell, a bacteria or other eukaryotic or prokaryotic cell. The mammalian cell may be in vivo or in vitro. Examples of a mammalian cell are an adrenal cell, a gonadal cell, a placental cell, a cell from the gastrointestinal tract, a kidney cell, a brain cell, or a skin cell. Furthermore, the enzyme system(s) may be a recombinant system in the cell.

In a related embodiment there are provided enzymatically hydroxylated cholecalciferol metabolites enzymatically produced by the enzyme system described herein.

In yet another embodiment of the present invention there is provided a hydroxylated cholecalciferol or ergocalciferol derivative or analog compound that is (5Z,7E)-9,10-secocholesta-5,7,10(19)-triene-3β,220-diol, (5Z,7E)-9,10-secocholesta-5,7,10(19)-triene-33,20,23-triol, (5Z,7E)-9,10-secocholesta-5,7,10(19)-triene-3β,17α,20,23-tetrol, (5Z,7E)-9,10- secochalesta-5,7,10(19)-triene-1a,3b,20,23-tetrol, or (5Z,7E)-9,10-secocholesta-5,7,10(19)-triene-1α,3β,17α,20,23-pentol (6E)-9,10-secocholesta-5(10),6,8-triene-3β,20α-diol, (6E)-9,10-secocholesta-5(10),6,8-triene-3b,20b-diol, 9b,10a-cholesta-5,7-diene-3β,20α-diol, 9b,10a-cholesta-5,7-diene-3β,20β-diol, cholesta-5,7-diene-3β,20α-diol, cholesta-5,7-diene-β,20β-diol, (5Z,7E,22E)-9,10-secoergosta-5,7,9(10),22-tetraene-3β,20α-diol, (5Z,7E,22E)-9,10-secoergosta-5,7,9(10),22-tetraene-β,20β-diol, (6E,22E)-9,10-secoergosta-5(10),6,8,22-tetraene-3β,20α-diol, (6E,22E)-9,10-secoergosta-5(10),6,8,22-tetraene-3β,20β-diol, 9b,10a- ergosta-5,7,22-triene-3β,20α-diol, 9b,10a-ergosta-5,7,22-triene-β,20β-diol, ergosta-5,7,22-triene-3β,20α-diol, or ergosta-5,7,22-triene-β,20β-diol.

Provided herein are a series of novel androsta-pregna-5,7-dienes and ergosta-5,7-dienes and 5,6,8-trienes and the corresponding ultraviolet B (UVB) irradiated 9,10-secosteroid products thereof. For example, the compounds may be, but are not limited to, secosteroidal, such as vitamin D-like, including vitamin-D3 (cholecalciferol) hydroxy derivatives, vitamin D2 (ergocalciferol) hydroxy derivatives and their luminosterol and tachysterol derivatives, analogs and epimers thereof. Preferably, the novel compounds of the present invention may be those identified in Table 1.

The series of androsta- and pregna-5,7-dienes were efficiently synthesized from their 3-acetylated 5-en precursors by bromination-dehydrobromination and deacetylation reactions. Ultraviolet B (UVB) irradiation was used to generate corresponding 9,10-secosteroids with vitamin D or D3-like, tachysterol-like (T-like) structures and 5,7-dienes with an inverted configuration at C-9 and C-10 that are lumisterol-like (L-like). Different doses of UVB resulted in formation of various products. At low doses, previtamin D-, T- or L-like compounds were formed as the main products, while higher doses induced predominantly the formation of vitamin D analogues with further isomerization thereof. It is contemplated that the ether and ester derivatives of these novel compounds can be produced by conventional chemical synthetic methods, methods which includes derivatizing the hydroxy and/or carbonyl moieties to produce the esters or ethers. Correspondingly, the ergosta-5,7-dienes and 5,6,8-trienes may be synthesized from their 3-acetylated 5-3en pregnenolone and 7DHP precursors via at least the same or similar bromination-dehydrobromination and deacetylation reactions with UV irradiation of the 20-OH-ergosterol product to yield the hydroxylated vitamin D2 derivative and the tachysterol-like and lumisterol-like analog structures.

Alternatively, methods of enzymatically synthesizing hydroxy derivatives of cholecalciferol (vitamin D3) and ergocalciferol (vitamin D2) using the cytochrome P450scc (CYP11A1) system or the CYP27b enzyme system, as described herein, are provided. It is contemplated that the hydroxylase may be any hydroxylase, e.g., plant or animal, including insect, hydroxylase that has an activity effective to hydroxylate position C20 of a secosteroid or its 5,7-dieneal precursor. These hydroxylated cholecalciferols include (5Z,7E)-9,10-secocholesta-5,7,10(19)-triene-3β,20-diol (20-hydroxycholecalciferol or 20-hydroxyvitamin D3), (5Z,7E)-9,10-secocholesta-5,7,10(19)-triene-3β,20,23-triol(20,23-dihydroxycholecalciferol or 20,23-dihydroxyvitamin D3), (5Z,7E)-9,10-secocholesta-5,7,10(19)-triene-3β,17α,20,23-tetrol (17α,20,23-trihydroxycholecalciferol or 17α,20,23-trihydroxyvitamin D3), (5Z,7E)-9,10-secocholesta-5,7,10(19)-triene-1α,3β,20,23-tetrol (1α,20,23-trihydroxycholecalciferol or 1α,20,23-trihydroxyvitamin D3), (5Z,7E)-9,10-secocholesta-5,7,10(19)-triene-1α,3β,17α,20,23-pentol (1α,17α,20,23-tetrahydroxycholecalciferol or 1α,17α,20,23-tetrahydroxyvitamin D3), or (5Z,7E)-9,10-secocholesta-5,7,10(19)-triene-1α,3β,20-triol (1α,20-dihydroxycholecalciferol or 1α,20-dihydroxyvitamin D3). The hydroxylated ergocalciferols include (6E)-9,10-secocholesta-5(10),6,8-triene-3β,20α-diol, (6E)-9,10-secocholesta-5(10),6,8-triene-3β,20β-diol, 9β,10α- cholesta-5,7-diene-3β,20α-diol, 9β,10α-cholesta-5,7-diene-3β,20β-diol, cholesta-5,7-diene-3β,20β-diol, cholesta-5,7-diene-β,20β-diol, (5Z,7E,22E)-9,10-secoergosta-5,7,9(10),22-tetraene-3β,20α-diol, (5Z,7E,22E)-9,10-secoergosta-5,7,9(10),22-tetraene-β,20β-diol, (6E,22E)-9,10-secoergosta-5(10),6,8,22-tetraene-3β,20α-diol, (6E,22E)-9,10-secoergosta-5(10),6,8,22-tetraene-3β,20β-diol, 9β,10α-ergosta-5,7,22-triene-3β,20α-diol, 9β,10α-ergosta-5,7,22-triene-β,20β-diol, ergosta-5,7,22-triene-3β,20α-diol, or ergosta-5,7,22-triene-β,20β-diol.

Furthermore, the methods of producing hydroxylated cholecalciferols may be utilized in vitro or in vivo. The enzyme systems may comprise a mammalian cell, a plant cell, an insect cell, a yeast cell or a bacterial cell or other eukaryotic or prokaryotic cells either in vitro or in vivo. For example mammalian cells having the ability to express CYP11A1 or CYP27B1 are, but not limited to, an adrenal cell, a gonadal cell, a placental cell, a cell from the gastrointestinal tract, a kidney cell, a brain cell, or a skin cell.

It is known that hydroxy-derivatives of plant derived ergosterol and ergocalciferol (vitamin D2), produced by the action of P450scc, have biological actions on skin cells cultured in vitro (7-8). It is demonstrated herein for the first time that products of vitamin D3 metabolism catalyzed by P450scc, 20-hydroxycholecalciferol, 20,23-dihydroxycholecalciferol, 17α,20,23-trihydroxycholecalciferol, and 1α,20-dihydroxycholecalciferol and products further catalyzed by CYP27B1, 1α,20,23-trihydroxycholecalciferol, 1α,17α,20,23-tetrahydroxycholecalciferol, act as an inhibitor of cell proliferation and a stimulator of keratinocyte differentiation, acting with comparable potency to calcitriol. Furthermore, its action on the expression of CYP27B1, CYP27A1 and CYP24 genes suggests a potential role in the regulation of calcitriol production, which may depend on the cell type used. 20-hydroxycholecalciferol is identified as a biologically active secosteroid that is a potent stimulator of epidermal keratinocyte differentiation. Thus, it is contemplated that secosteroids produced by P450scc in an alternate pathway of vitamin D3 or vitamin D2 metabolism to that for calcitriol synthesis (9-10) plays an important role in at least cutaneous biology.

Generally, the present invention also provides methods of treating or improving a condition associated with proliferating cells, either normally proliferating cells or abnormally or uncontrolled proliferating, e.g., neoplastic, cells. In addition to those novel compounds identified in Table 1 and the ether and ester derivatives thereof, the following compounds listed in Table 2 may have an antiproliferative or other therapeutic effect on the condition or may improve the cosmetic appearance of the cells, may have a prophylatic action thereon or may maintain the health of the cells and the subject.

TABLE 1

| No | Short name | Name | UV max (nm) | Pred. MW |
|---|---|---|---|---|
| 1T | pT3 | (6E)-3β-hydroxy-9,10-secopregna-5(10),6,8-trien-20-one | 274, 287, 290 | 314.46 |
| 2L | 17α-OH pL3 | 3β,17α-dihydroxy-9β,10α-pregna-5,7-dien-20-one | 264, 273, 281 | 330.46 |
| 2T | 17α-OH pT3 | (6E)-3β,17α-dihydroxy-9,10-secopregna-5(10),6,8-trien-20-one | 271, 280, 290 | 330.46 |
| 3 | 20(OH)7DHC | cholesta-5,7-diene-3β,20α or β-diol | | |
| 3D | 20(OH)D3 | (5Z,7E)-9,10-secocholesta-5,7,9(10)-triene-3β,20-diol | | |
| 3L | 20-OH pL3 | 3β,20-dihydroxy-9β,10α-pregna-5,7-diene | 262, 272, 281 | 316.48 |
| 3cL | 20(OH)cL3 | 9b,10α-cholesta-5,7-diene-3β,20-diol | | |
| 3T | 20-OH pT3 | (6E)-9,10-secopregna-5(10),6,8-triene-3β,20-diol | 272, 280, 291 | 316.48 |
| 3cT | 20(OH)cT3 | (6E)-9,10-secocholesta-5(10),6,8-triene-3β,20-diol | | |
| 4L | aL3 | 9β,10α-androsta-5,7-dien-3β-ol | 261, 272, 280 | 286.41 |
| 4T | aT3 | (6E)-9,10-secoandrosta-5(10),6,8-trien-3β-ol | 271, 280, 289 | 286.41 |
| 5D | 17(OH)D3 | (5Z,7E)-9,10-secocholesta-5,7,9(10)-triene-3β,17α-diol | | |
| 5L | 17α-OH aL3 | 9β,10α-androsta-5,7-diene-3β,17α-diol | 262, 271, 282 | 288.42 |
| 5T | 17α-OH aT3 | (6E)-9,10-secoandrosta-5(10),6,8-triene-3β,17α-diol | 272, 281, 291 | 288.42 |
| 6D | 17α,20S-diOH pD3 | (5Z,7E)-(20S)-9,10-secopregna-5,7,10(19)-triene-3β,17α,20-triol | 265 | 332.47 |
| 6L | 17α,20S-diOH pL3 | (20S)-9β,10α-pregna-5,7-diene-3β,17α,20-triol | 262, 271, 282 | 332.47 |
| 6T | 17α,20S-diOH pT3 | (6E)-(20S)-9,10-secopregna-5(10),6,8-triene-3β,17α 20-triol | 272, 282, 297 | 332.47 |
| 6TR | 17α,20S-diOH 5,7,9DHP | (20S)-pregna-5,7,9(11)-triene-3β,17α,20-triol | 312, 323, 340 | 332.47 |
| 7D | 17α,20R-diOH pD3 | (5Z,7E)-(20R)-9,10-secopregna-5,7,10(19)-triene-3β,17α,20-triol | 265 | 332.47 |
| 7L | 17α,20R-diOH pL3 | (20R)-9β,10α-pregna-5,7-diene-3β,17α,20-triol | 262, 271, 281 | 332.47 |
| 7T | 17α,20R-diOH pT3 | (6E)-(20R)-9,10-secopregna-5(10),6,8-triene-3β,17α,20-triol | 270, 279, 291 | 332.47 |
| 8 | 11α,20R-diOH 7DHP | (20R)-pregna-5,7-diene-3β,11α,20-triol | | 332.47 |
| 8D | 11α,20R-diOH pD3 | (5Z,7E)-(20R)-9,10-secopregna-5,7,10(19)-triene-3β,11α,20-triol | | 332.47 |
| 8L | 11α,20R-diOH pL3 | (20R)-9β,10α-pregna-5,7-diene-3β,11α,20-triol | | 332.47 |
| 8T | 11α,20R-diOH pT3 | (6E)-(20R)-trihydroxy-9,10-secopregna-5(10),6,8-triene-3β,11α,20- | | 332.47 |
| | | triol | | |
| 9 | 11α,20S-diOH 7DHP | (20S)-pregna-5,7-diene-3β,11α,20-triol | | 332.47 |
| 9D | 11α,20S-diOH pD3 | (5Z,7E)-(20S)-9,10-secopregna-5,7,10(19)-triene-3β,11α,20-triol | | 332.47 |
| 9L | 11α,20S-diOH pL3 | (20S)-9β,10α-pregna-5,7-diene-3β,11α,20-triol | | 332.47 |
| 9T | 11α,20S-diOH pT3 | (6E)-(20S)-9,10-secopregna-5(10),6,8-triene-3β,11α,20-triol | | 332.47 |
| 10 | 11β,20R-diOH 7DHP | (20R)-pregna-5,7-diene-3β,11β,20-triol | | 332.47 |
| 10D | 11β,20R-diOH pD3 | (5Z,7E)-(20R)-9,10-secopregna-5,7,10(19)-triene-3β,11β,20-triol | | 332.47 |
| 10L | 11β,20R-diOH pL3 | (20R)-9β,10α-pregna-5,7-diene-3β,11β,20-triol | | 332.47 |
| 10T | 11β,20R-diOH pT3 | (6E)-(20R)-9,10-secopregna-5(10),6,8-triene-3β,11β,20-triol | | 332.47 |
| 11 | 11β,20S-diOH 7DHP | (20S)-pregna-5,7-diene-3β,11β,20-triol | | 332.47 |
| 11D | 11β,20S-diOH pD3 | (5Z,7E)-(20S)-9,10-secopregna-5,7,10(19)-triene-3β,11β,20-triol | | 332.47 |
| 11L | 11β,20S-diOH pL3 | (20S)-9β,10α-pregna-5,7-diene-3β,11β,20-triol | | 332.47 |
| 11T | 11β,20S-diOH pT3 | (6E)-(20S)-9,10-secopregna-5(10),6,8-triene-3β,11β,20-tetrol | | 332.47 |
| 12 | 11α,17α,20R-triOH 7DHP | (20R)-pregna-5,7-diene-3β,11α,17α,20-tetrol | | 348.48 |
| 12D | 11β,17α,20R-triOH pD3 | (5Z,7E)-(20R)-9,10-secopregna-5,7,10(19)-triene-3β,11α,17α,20- | | 348.48 |
| | | tetrol | | |
| 12L | 11α,17α,20R-triOH pL3 | (20R)-9β,10α-pregna-5,7-diene-3β,11α,17α,20-tetrol | | 348.48 |
| 12T | 11α,17α,20R-triOH pT3 | (6E)-(20R)-9,10-secopregna-5(10),6,8-triene-3β,11α,17α,20-tetrol | | 348.48 |
| 13 | 11α,17α,20S-triOH 7DHP | (20S)-pregna-5,7-diene-3β,11α,17α,20-tetrol | | 348.48 |
| 13D | 11α,17α,20S-triOH pD3 | (5Z,7E)-(20S)-9,10-secopregna-5,7,10(19)-triene-3β,11α,17α,20- | | 348.48 |
| | | tetrol | | |
| 13L | 11α,17α,20S-triOH pL3 | (20S)-9β,10α-pregna-5,7-diene-3β,11α,17α,20-tetrol | | 348.48 |
| 13T | 11α,17α,20S-triOH pT3 | (6E)-(20S)-9,10-secopregna-5(10),6,8-triene-3β,11α,17α,20-tetrol | | 348.48 |
| 14 | 11β,17α,20R-triOH 7DHP | (20R)-pregna-5,7-diene-3β,11β,17α,20-tetrol | | 348.48 |
| 14D | 11β,17α,20R-triOH pD3 | (5Z,7E)-(20R)-9,10-secopregna-5,7,10(19)-triene-3β,11β,17α,20- | | 348.48 |
| | | tetrol | | |
| 14L | 11β,17α,20R-triOH pL3 | (20R)-9β,10α-pregna-5,7-diene-3β,11β,17α,20-tetrol | | 348.48 |
| 14T | 11β,17α,20R-triOH pT3 | (6E)-(20R)-9,10-secopregna-5(10),6,8-triene-3β,11β,17α,20-tetrol | | 348.48 |
| 15 | 11β,17α,20S-triOH 7DHP | (20S)-tetrahydroxypregna-5,7-diene-3β,11β,17α,20-tetrol | | 348.48 |
| 15D | 11β,17α,20S-triOH pD3 | (5Z,7E)-(20S)-9,10-secopregna-5,7,10(19)-triene-3β,11β,17α,20- | | 348.48 |
| | | tetrol | | |
| 15L | 11β,17α,20S-triOH pL3 | (20S)-9β,10α-pregna-5,7-diene-3β,11β,17α,20S-tetrol | | 348.48 |
| 15T | 11β,17α,20S-triOH pT3 | (6E)-(20S)-9,10-secopregna-5(10),6,8-triene-3β,11β,17α,20-tetrol | | 348.48 |
| 16 | 20R,21-diOH 7DHP | 3β,20,21-pregna-5,7-diene-3β,20,21-triol | | 332.48 |
| 16D | 20R,21-diOH pD3 | (5Z,7E)-(20R)-9,10-secopregna-5,7,10(19)triene-3β,20,21-triol | | 332.48 |
| 16L | 20R,21-diOH pL3 | (20R)-9β,10α-pregna-5,7-diene-3β,20,21-triol | | 332.48 |
| 16T | 20R,21-diOH pT3 | (6E)-(20R)-9,10-secopregna-5(10),6,8-triene-3β,20,21-triol | | 332.48 |
| 17 | 20S,21-diOH 7DHP | (20S)-pregna-5,7-diene-3β,20,21-triol | | 332.48 |
| 17D | 20S,21-diOH pD3 | (5Z,7E)-(20S)-9,10-secopregna-5,7,10(19)-triene-3β,20,21-triol | | 332.48 |
| 17L | 20S,21-diOH pL3 | (20S)-9β,10α-pregna-5,7-diene-3β,20,21-triol | | 332.48 |
| 17T | 20S,21-diOH pT3 | (6E)-(20S)-9,10-secopregna-5(10),6,8-triene-3β,20,21-triol | | 332.48 |
| 18 | 17α,20R,21-triOH 7DHP | (20R)-pregna-5,7-diene-3β,17α,20,21-tetrol | | 348.48 |
| 18D | 17α,20R,21-triOH pD3 | (5Z,7E)-(20R)-9,10-secopregna-5,7,10(19)-triene-3β,17α,20,21- | | 348.48 |
| | | tetrol | | |
| 18L | 17α,20R,21-triOH pL3 | (20R)-9β,10α-pregna-5,7-diene-3β,17α,20,21-tetrol | | 348.48 |
| 18T | 17α,20R,21-triOH pT3 | (6E)-(20R)-9,10-secopregna-5(10),6,8-triene-3β,17α,20,21-tetrol | | 348.48 |
| 19 | 17α,20S,21-triOH 7DHP | (20S)-pregna-5,7-diene-3β,17α,20,21-tetrol | | 348.48 |
| 19D | 17α,20S,21-triOH pD3 | (5Z,7E)-(20S)-9,10-secopregna-5,7,10(19)-triene-3β,17α,20,21- | | 348.48 |
| | | tetrol | | |
| 19L | 17α,20S,21-triOH pL3 | (20S)-9β,10α-pregna-5,7-diene-3β,17α,20,21-tetrol | | 348.48 |
| 19T | 17α,20S,21-triOH pT3 | (6E)-(20R)-9,10-secopregna-5(10),6,8-triene-3β,17α,20,21-tetrol | | 348.48 |
| 20 | 11α,20R,21-triOH 7DHP | (20R)-pregna-5,7-diene-3β,11α,20,21-tetrol | | 348.48 |
| 20D | 11α,20R,21-triOH pD3 | (5Z,7E)-(20R)-9,10-secopregna-5,7,10(19)-triene-3β,11α,20,21- | | 348.48 |
| | | tetrol | | |
| 20L | 11α,20R,21-triOH pL3 | (20R)-9β,10α-pregna-5,7-diene-3β,11α,20,21-tetrol | | 348.48 |
| 20T | 11α,20R,21-triOH pT3 | (6E)-(20R)-9,10-secopregna-5(10),6,8-triene-3β,11α,20,21-tetrol | | 348.48 |
| 21 | 11α,20S,21-triOH 7DHP | (20S)-tetrahydroxypregna-5,7-diene-3β,11α,20,21-tetrol | | 348.48 |
| 21D | 11α,20S,21-triOH pD3 | (5Z,7E)-(20S)-9,10-secopregna-5,7,10(19)-triene-3β,11α,20,21- | | 348.48 |
| | | tetrol | | |
| 21L | 11α,20S,21-triOH pL3 | (20S)-9β,10α-pregna-5,7-diene-3β,11α,20,21-tetrol | | 348.48 |
| 21T | 11α,20S,21-triOH pT3 | (6E)-(20S)-9,10-secopregna-5(10),6,8-triene-3β,11α,20,21-tetrol | | 348.48 |
| 22 | 11β,20R,21-triOH 7DHP | (20R)-pregna-5,7-diene-3β,11β,20,21-tetrol | | 348.48 |
| 22D | 11β,20R,21-triOH pD3 | (5Z,7E)-(20R)-9,10-secopregna-5,7,10(19)-triene-3β,11β,20,21- | | 348.48 |
| | | tetrol | | |
| 22L | 11β,20R,21-triOH pL3 | (20R)-9β,10α-pregna-5,7-diene-3β,11β,20,21-tetrol | | 348.48 |
| 22T | 11β,20R,21-triOH pT3 | (6E)-(20R)-9,10-secopregna-5(10),6,8-triene-3β,11β,20,21-tetrol | | 348.48 |
| 23 | 11β,20S,21-triOH 7DHP | (20S)-pregna-5,7-diene-3β,11β,20,21-tetrol | | 348.48 |
| 23D | 11β,20S,21-triOH pD3 | (5Z,7E)-(20S)-9,10-secopregna-5,7,10(19)-triene-3β,11β,20,21-tetrol | | 348.48 |
| 23L | 11β,20S,21-triOH pL3 | (20S)-9β,10α-pregna-5,7-diene-3β,11β,20,21-tetrol | | 348.48 |
| 23T | 11β,20S,21-triOH pT3 | (6E)-(20S)-9,10-secopregna-5(10),6,8-triene-3β,11β,20,21-tetrol | | 348.48 |
| 24 | 11α,17α,20R,21-tetraOH | (20R)-pregna-5,7-diene-3β,11α,17α,20,21-pentol | | 364.48 |
| | 7DHP | | | |
| 24D | 11α,17α,20R,21-tetraOH | (5Z,7E)-(20R)-9,10-secopregna-5,7,10(19)-triene- | | 364.48 |
| | pD3 | 3β,11α,17α,20,21-pentol | | |
| 24L | 11α,17α,20R,21-tetraOH | (20R)-9β,10α-pregna-5,7-diene-3β,11α,17α,20,21-pentol | | 364.48 |
| | pL3 | | | |
| 24T | 11α,17α,20R,21-tetraOH | (6E)-(20R)-9,10-secopregna-5(10),6,8-triene-3β,11α,17α,20,21- | | 364.48 |
| | pT3 | pentol | | |
| 25 | 11α,17α,20S,21-tetraOH | (20S)-pregna-5,7-diene-3β,11α,17α,20,21-pentol | | 364.48 |
| | 7DHP | | | |
| 25D | 11α,17α,20S,21-tetraOH | (5Z,7E)-(20S)-9,10-secopregna-5,7,10(19)-triene- | | 364.48 |
| | pD3 | 3β,11α,17α,20,21-pentol | | |
| 25L | 11α,17α,20S,21-tetraOH | (20S)-9β,10α-pregna-5,7-diene-3β,11α,17α,20,21-pentol | | 364.48 |
| | pL3 | | | |
| 25T | 11α,17α,20S,21-tetraOH | (6E)-_(20S)-9,10-secopregna-5(10),6,8-triene-3β,11α,17α,20,21- | | 364.48 |
| | pT3 | pentol | | |
| 26 | 11β,17α,20R,21-tetraOH | (20R)-pregna-5,7-diene-3β,11α,17α,20,21-pentol | | 364.48 |
| | 7DHP | | | |
| 26D | 11β,17α,20R,21-tetraOH | (5Z,7E)-(20R)-9,10-secopregna-5,7,10(19)-triene- | | 364.48 |
| | pD3 | 3β,11α,17α,20,21-pentol | | |
| 26L | 11β,17α,20R,21-tetraOH | (20R)-9β,10α-pregna-5,7-diene-3β,11α,17α,20,21-pentol | | 364.48 |
| | pL3 | | | |
| 26T | 11β,17α,20R,21-tetraOH | (6E)-(20R)-9,10-secopregna-5(10),6,8-triene-3β,11α,17α,20,21- | | 364.48 |
| | pT3 | pentol | | |
| 27 | 11β,17α,20S,21-tetraOH | (20S)-pregna-5,7-diene-3β,11β,17α,20,21-pentol | | 364.48 |
| | 7DHP | | | |
| 27D | 11β,17α,20S,21-tetraOH | (5Z,7E)-(20S)-9,10-secopregna-5,7,10(19)-triene- | | 364.48 |
| | pD3 | 3β,11β,17α,20,21-pentol | | |
| 27L | 11β,17α,20S,21-tetraOH | (20S)-9β,10α-pregna-5,7-diene-3β,11β,17α,20,21-pentol | | 364.48 |
| | pL3 | | | |
| 27T | 11β,17_,20S,21-tetraOH | (6E)-(20S)-9,10-secopregna-5(10),6,8-triene-3β,11β,17α,20,21- | | 364.48 |
| | pT3 | pentol | | |
| 28 | 11α,17α-diOH 7DHEA | androsta-5,7-diene-3β,11α,17α-triol | | 304.42 |
| 28D | 11α,17α-diOH aD3 | (5Z,7E)-9,10-secoandrosta-5,7,10(19)-triene-3β,11α,17α-triol | | 304.42 |
| 28L | 11α,17α-diOH aL3 | 9β,10α-androsta-5,7-diene-3β,11α,17α-triol | | 304.42 |
| 28T | 11α,17α-diOH aT3 | (6E)-9,10-secoandrosta-5(10),6,8-triene-3β,11α,17α-triol | | 304.42 |
| 29 | 11β,17α-diOH 7DHEA | androsta-5,7-diene-3β,11β,17α-triol | | 304.42 |
| 29D | 11β,17α-diOH aD3 | (5Z,7E)-9,10-secoandrosta-5,7,10(19)-triene-3β,11β,17α-triol | | 304.42 |
| 29L | 11β,17α-diOH aL3 | 9β,10α-androsta-5,7-diene-3β,11β,17α-triol | | 304.42 |
| 29T | 11β,17α-diOH aT3 | (6E)-9,10-secoandrosta-5(10),6,8-triene-3β,11β,17α-triol | | 304.42 |
| 30 | 11α-OH 7DHEA | androsta-5,7-diene-3β,11α-diol | | 302.41 |
| 30D | 11α-OH aD3 | (5Z,7E)-9,10-secoandrosta-5,7,10(19)-triene-3β,11α-diol | | 302.41 |
| 30L | 11α-OH aL3 | 9β,10α-androsta-5,7-diene-3β,11α-diol | | 302.41 |
| 30T | 11α-OH aT3 | (6E)-9,10-secoandrosta-5(10),6,8-triene-3β,11α-diol | | 302.41 |
| 31 | 11β-OH 7DHEA | androsta-5,7-diene-3β,11β-diol | | 302.41 |
| 31D | 11β-OH aD3 | (5Z,7E)-9,10-secoandrosta-5,7,10(19)triene-3β,11β-diol | | 302.41 |
| 31L | 11β-OH aL3 | 9β,10α-androsta-5,7-diene-3β,11β-diol | | 302.41 |
| 31T | 11β-OH aT3 | (6E)-9,10-secoandrosta-5(10),6,8-triene-3β,11β-diol | | 302.41 |
| 32L | 11α-OH pL3 | 3β,11α-dihydroxy-9β,10α-pregna-5,7-dien-20-one | | 330.46 |
| 32T | 11α-OH pT3 | (6E)-3β,11α-dihydroxy-9,10-secopregna-5(10),6,8-trien-20-one | | 330.46 |
| 33L | 11β-OH pL3 | 3β,11β-dihydroxy-9β,10α-pregna-5,7-dien-20-one | | 330.46 |
| 33T | 11β-OH pT3 | (6E)-3β,11β-dihydroxy-9,10-secopregna-5(10),6,8-trien-20-one | | 330.46 |
| 34L | 11α,17α-diOH pL3 | 3β,11α,17α-trihydroxy-9β,10α-pregna-5,7-dien-20-one | | 346.46 |
| 34T | 11α,17α-diOH pT3 | (6E)-3β,11α,17α-trihydroxy-9,10-secopregna-5(10),6,8-trien-20-one | | 346.46 |
| 35L | 11β,17α-diOH pL3 | 3β,11β,17α-trihydroxy-9β,10α-pregna-5,7-dien-20-one | | 346.46 |
| 35T | 11β,17α-diOH pT3 | (6E)-3β,11β,17α-trihydroxy-9,10-secopregna-5(10),6,8-trien-20-one | | 346.46 |
| 36L | 21-OH pL3 | 3β,21-dihydroxy-9β,10α-pregna-5,7-dien-20-one | | 330.46 |
| 36T | 21-OH pT3 | (6E)-3β,21-dihydroxy-9,10-secopregna-5(10),6,8-trien-20-one | | 330.46 |
| 37L | 17α,21-diOH pL3 | 3β,17α,21-trihydroxy-9β,10α-pregna-5,7-dien-20-one | | 346.46 |
| 37T | 17α,21-diOH pT3 | (6E)-3β,17α,21-trihydroxy-9,10-secopregna-5(10),6,8-trien-20-one | | 346.46 |
| 38c | 11α,21-diOH 7DHP | 3β,11α,21-trihydroxypregna-5,7-dien-20-one | | 346.46 |
| 38L | 11α,21-diOH pL3 | 3β,11α,21-trihydroxy-9β,10α-pregna-5,7-dien-20-one | | 346.46 |
| 38T | 11α,21-diOH pT3 | (6E)-3β,11α,21-trihydroxy-9,10-secopregna-5(10),6,8-trien-20-one | | 346.46 |
| 39L | 11β,21-diOH pL3 | 3β,11β,21-trihydroxy-9β,10α-pregna-5,7-dien-20-one | | 346.46 |
| 39T | 11β,21-diOH pT3 | (6E)-3β,11β,21-trihydroxy-9,10-secopregna-5(10),6,8-trien-20-one | | 346.46 |
| 40L | 11α,17α,21-triOH pL3 | 3β,11α,17α,21-tetrahydroxy-9β,10α-pregna-5,7-dien-20-one | | 362.46 |
| 40T | 11α,17α,21-triOH pT3 | (6E)-3β,11α,17α,21-tetrahydroxy-9,10-secopregna-5(10),6,8-trien- | | 362.46 |
| | | 20-one | | |
| 41L | 11β,17α,21-triOH pL3 | 3β,11β,17α,21-tetrahydroxy-9β,10α-pregna-5,7-dien-20-one | | 362.46 |
| 41T | 11β,17α,21-triOH pT3 | (6E)-3β,11β,17α,21-tetrahydroxy-9,10-secopregna-5(10),6,8-trien- | | 362.46 |
| | | 20-one | | |
| 42D | 1α,20-diOH D3 | (5Z,7E)-9,10-secocholesta-5,7,10(19)-triene-1α,3β,20-triol | | |
| 43D | 23(OH)D3 | (5Z,7E)-9,10-secocholesta-5,7,9(10)-triene-3β,23-diol | | |
| 43L | 23(OH) pL3 | 3β,23-dihydroxy-9β,10α-pregna-5,7-dien-20-one | | |
| 43T | 23(OH) pT3 | (6E)-3β,23-dihydroxy-9,10-secopregna-5(10),6,8-trien-20-one | | |
| 44D | 20,23-diOH D3 | (5Z,7E)-9,10-secocholesta-5,7,10(10)-triene-3β,20,23-triol | | |
| 44L | 20,23-diOH pL3 | 3β,20α or 20β,23-trihydroxy-9β,10α-pregna-5,7-dien-20-one | | |
| 44T | 20,23-diOH pT3 | (6E)-3β,20α or 20β,23-trihydroxy-9,10-secopregna-5(10),6,8-trien- | | |
| | | 20-one | | |
| 45D | 1α,20,23-triOH D3 | (5Z,7E)-9,10-secocholesta-5,7,10(10)-triene-1α,3β,20,23-tetrol | | |
| 45L | 1α,20,23-triOH pL3 | 1α,3β,20α or 20β,23-tetrahydroxy-9β,10α-pregna-5,7-dien-20-one | | |
| 45T | 1α,20,23-triOH pT3 | (6E)-1α,3β,20α or 20β,23-tetrahydroxy-9,10-secopregna-5(10),6,8- | | |
| | | trien-20-one | | |
| 46D | 17α,20-diOHD3 | (5Z,7E)-9,10-secocholesta-5,7,9(10)-triene-3β,17α,20-triol | | |
| 46L | 17α,20-diOH pL3 | 3β,17α,20α or 20β-trihydroxy-9β,10α-pregna-5,7-dien-20-one | | |
| 46T | 17α,20-diOH pT3 | (6E)-3β,17α,20α or 20β-trihydroxy-9,10-secopregna-5(10),6,8-trien- | | |
| | | 20-one | | |
| 47D | 17α,20,23-triOH D3 | (5Z,7E)-9,10-secocholesta-5,7,10(19)-triene-3β,17α,20,23-tetrol | | |
| 47L | 17α,20,23-triOH pL3 | 3β,17α,20α or 20β,23-tetrahydroxy-9β,10α-pregna-5,7-dien-20-one | | |
| 47T | 17α,20,23-triOH pT3 | (6E)-3β,17α,20α or 20β,23-tetrahydroxy-9,10-secopregna- | | |
| | | 5(10),6,8-trien-20-one | | |
| 48D | 1α,17α,20-triOHD3 | (5Z,7E)-9,10-secocholesta-5,7,9(10)-triene-1α,3β,17α,20- | | |
| | | tetrol | | |
| 48L | 1α,17α,20-triOH pL3 | 1α,3β,17α,20α or 20β-tetrahydroxy-9β,10α-pregna-5,7-dien-20-one | | |
| 48T | 1α,17α,20-triOH pT3 | (6E)-1α,3β,17α,20α or 20β-tetrahydroxy-9,10-secopregna- | | |
| | | 5(10),6,8-trien-20-one | | |
| 49D | 1α,17α,20,23-tetraOHD3 | (5Z,7E)-9,10-secocholesta-5,7,10(19)-triene-1α,3β,17α,20,23- | | |
| | | pentol | | |
| 48L | 1α,17α,20,23-tetraOH pL3 | 1α,3β,17α,20α or 20β,23-pentahydroxy-9β,10α-pregna-5,7-dien-20- | | |
| | | one | | |
| 48T | 1α,17α,20,23-tetraOHpT3 | (6E)-1α,3β,17α,20α or 20β,23-pentahydroxy-9,10-secopregna- | | |
| | | 5(10),6,8-trien-20-one | | |
| 50A | 17-COOH | (5Z,7E)-3β-hydroxy-androsta-5,7-diene-17β-carboxylic acid | 274, 285 | 315 [M − H]⁻ |
| 51D3 | 17-COOH aD3 | (5Z,7E)-3β-hydroxy-9,10-secoandrosta-5,7,9(10)-triene-17β- | | 315 [M − H]⁻ |
| | | carboxylic acid | | |
| 52T3 | 17-COOH aT3 | (6E)-3β-hydroxy-9,10-secoandrosta-5(10),6,8-triene-17β-carboxylic | | 315 [M − H]⁻ |
| | | acid | | |
| 53L3 | 17-COOH aL3 | (5Z,7E)-3β-hydroxy-9β,10α-androsta-5,7-diene-17β-carboxylic acid | | 315 [M − H]⁻ |
| 54D2 | 20(OH)D2 | (5Z,7E,22E)-9,10-secoergosta-5,7,9(10),22-tetraene-3β,20- | | |
| | | diol | | |
| 54eT2 | 20(OH)eT2 | (6E,22E)-9,10-secoergosta-5(10),6,8,22-tetraene-3β,20-diol | | |
| 54eL2 | 20(OH)eL2 | 9β,10α-ergosta-5,7,22-triene-3β,20-diol | | |
| 54e | 20(OH)ergosterol | ergosta-5,7,22-triene-3β,20-diol | | |

TABLE 2

| No | Short name | Name | UV max (nm) | Predicted MW |
|---|---|---|---|---|
| 1 | 7DHP | 3β-hydroxypregna-5,7-dien-20-one | 262, 272, 283, 294 | 314.46 |
| 1D | pD3 | (5Z,7E)-3β-hydroxy-9,10-secopregna-5,7,10(19)-trien-20- | 265 | 314.46 |
| | | one | | |
| 1L | pL3 | 3β-hydroxy-9β,10α-pregna-5,7-dien-20-one | ND | 314.46 |
| 2 | 17α-OH 7DHP | 3β,17α-dihydroxypregna-5,7-dien-20-one | 263, 272, 281, 293 | 330.46 |
| 2pD | 17α-OH pD3 | (5Z,7E)-3β,17α-dihydroxy-9,10-secopregna-5,7,10(19)-trien- | 265 | 330.46 |
| | | 20-one | | |
| 3 | 20-OH 7DHP | pregna-5,7-diene-3β,20-diol | 263, 272, 282, 293 | 316.48 |
| 3pD | 20-OH pD3 | (5Z,7E)-9,10-secopregna-5,7,10(19)-triene-3β,20-diol | 265 | 316.48 |
| 4 | 7DHEA | androsta-5,7-dien-3β-ol | 263, 271, 282, 292 | 286.41 |
| 4D | aD3 | (5Z,7E)-9,10-secoandrosta-5,7,10(19)-trien-3β-ol | 264 | 286.41 |
| 5 | 17α-OH 7DHEA | androsta-5,7-diene-3β,17α-diol | 262, 272, 281, 292 | 288.42 |
| 5D | 17α-OH aD3 | (5Z,7E)-9,10-secoandrosta-5,7,10(19)-triene-3β,17α-diol | 264 | 288.42 |
| 6 | 17α,20S-diOH 7DHP | (20S)-pregna-5,7-diene-3β,17α-triol | 263, 271, 282, 292 | 332.47 |
| 7 | 17α,20R-diOH 7DHP | (20R)-trihydroxypregna-5,7-diene-3β,17α,20-triol | 261, 270, 281, 290 | 332.47 |
| 7TR | 17α,20R-diOH | (20R)-trihydroxypregna-5,7,9(11)-triene-3β,17α,20-triol | 312, 323, 340 | 330.47 |
| | 5,7,9DHP | | | |
| 32 | 11α-OH 7DHP | 3β,11α-dihydroxypregna-5,7-dien-20-one | | 330.46 |
| 32D | 11α-OH pD3 | (5Z,7E)-3β,11α-dihydroxy-9,10-secopregna-5,7,10(19)-trien- | | 330.46 |
| | | 20-one | | |
| 33 | 11β-OH 7DHP | 3β,11β-dihydroxypregna-5,7-dien-20-one | | 330.46 |
| 33D | 11β-OH pD3 | (5Z,7E)-3β,11β-dihydroxy-9,10-secopregna-5,7,10(19)-trien- | | 330.46 |
| | | 20-one | | |
| 34 | 11α,17α-diOH 7DHP | 3β,11α,17α-trihydroxypregna-5,7-dien-20-one | | 346.46 |
| 34D | 11α,17α-diOH pD3 | (5Z,7E)-3β,11α,17α-trihydroxy-9,10-secopregna-5,7,10(19)- | | 346.46 |
| | | trien-20-one | | |
| 35 | 11β,17α-diOH 7DHP | 3β,11β,17α-trihydroxypregna-5,7-dien-20-one | | 346.46 |
| 35D | 11β,17α-diOH pD3 | (5Z,7E)-3β,11β,17α-trihydroxy-9,10-secopregna-5,7,10(19)- | | 346.46 |
| | | trien-20-one | | |
| 36 | 21-OH 7DHP | 3β,21-dihydroxy-5,7-dien-20-one | | 330.46 |
| 36D | 21-OH pD3 | (5Z,7E)-3β,21-dihydroxy-9,10-secopregna-5,7,10(19)-trien- | | 330.46 |
| | | 20-one | | |
| 37 | 17α,21-diOH 7DHP | 3β,17α,21-trihydroxy-5,7-dien-20-one | | 346.46 |
| 37D | 17α,21-diOH pD3 | (5Z,7E)-3β,17α,21-trihydroxy-9,10-secopregna-5,7,10(19)- | | 346.46 |
| | | trien-20-one | | |
| 38D* | 11α,21-diOH pD3 | (5Z,7E)-3β,11α,21-trihydroxy-9,10-secopregna-5,7,10(19)- | | 346.46 |
| | | trien-20-one | | |
| 39c | 11β,21-diOH 7DHP | 3β,11β,21-trihydroxypregna-5,7-dien-20-one | | 346.46 |
| 39D* | 11β,21-diOH pD3 | (5Z,7E)-3β,11β,21-trihydroxy-9,10-secopregna-5,7,10(19)- | | 346.46 |
| | | trien-20-one | | |
| 40 | 11α,17α,21-triOH | 3β,11α,17α,21-tetrahydroxypregna-5,7-dien-20-one | | 362.46 |
| | 7DHP | | | |
| 40D | 11α,17α,21-triOH pD3 | (5Z,7E)-3β,11α,17α,21-tetrahydroxy-9,10-secopregna- | | 362.46 |
| | | 5,7,10(19)-trien-20-one | | |
| 41 | 11β,17α,21-triOH | 3β,11β,17α,21-tetrahydroxypregna-5,7-dien-20-one | | 362.46 |
| | 7DHP | | | |
| 41D | 11β,17α,21-triOH pD3 | (5Z,7E)-3β,11β,17α,21-tetrahydroxy-9,10-secopregna- | | 362.46 |
| | | 5,7,10(19)-trien-20-one | | |

More particularly, the abnormally or uncontrollably proliferating cell may be malignant or benign neoplastic cells. For example, the antiproliferative action against human melanoma cells or melanocytes and keratinocytes, which are epithelial cells, demonstrated herein is indicative of an antiproliferative action against neoplastic cells comprising the epithelium, the breast, the genitourinary tract, the respiratory tract, the prostate, the endocrine system, the musculoskeletal and connective tissue systems, the vascular system, the hematologic system, the nervous system, the skin, or the immune system. These abnormal cells may be adrenal cell, a gonadal cell, a pancreatic cell, a cell from the gastrointestinal tract, a prostate cell, a breast cell, a lung cell, an immune cell, a hematologic cell, a kidney cell, a brain cell, a cell of neural crest origin, or a skin cell. The neoplastic cells may comprise a melanoma such as a melanocytic tumor or a melanoma of the skin, the eye or of an undetermined primary site. Also, the antiproliferative action against human leukemia cells is indicative of an action against a leukemia, such as, but not limited to chronic myeloid leukemia. In addition, the neoplastic cells may comprise a prostate carcinoma.

In addition, it is contemplated that the antiproliferative and anti-inflammatory action against keratinocytes is indicative that the cell may comprise a skin or mucosal disorder, such as, but not limited to, a hyperproliferative skin disorder, a pigmentary skin disorder, an inflammatory or autoimmune skin disorder, or other skin disorder. A hyperproliferative skin disorder may be psoriasis, seborrheic keratosis, actinic keratosis, benign adnexal tumor, fribromatosis, or keloids. A pigmentary skin disorder may be vitiligo, solar lentigo, lentigo simplex, hypermelanosis, or dysplastic melanocytic nevus. An inflammatory or autoimmune skin disorder may be allergic contact dermatitis, mummular dermatitis, atopic dermatitis, irritant contact dermatitis, or seborrheic dermatitis, pemphigus, bullous pemphigoid, or lupus erythematosus.

Other skin disorders may be alopecia of the scalp or a disorder encompassing overproduction of hair on the legs, arms, torso or face. Alternatively, in addition, a skin disorder may be induced by exposure to solar radiation. For example, aging of the skin, skin damage or a pre-carcinogenic or carcinogenic state is caused by this exposure. It is contemplated that the action of the compounds provided herein may be useful in controlling, attenuating or preventing aging of the skin.

It is further contemplated that the compounds and/or pharmaceutical compositions provided herein have cosmetic and/or prophylactic utilities. These compounds and compositions may counteract aging in general, for example, aging of certain internal organs, and skin aging in particular, carcinogenesis, hair growth abnormalities, depigmentation or hyperpigmentation, or allergic reactions. Also, the disclosed compounds and compositions are effective to prevent or delay development of skin pathologies and pathologies affecting cardiovascular system, central nervous system, endocrine system, immune system, reproductive system, gastrointestinal system, skeletomuscular system, adipose tissue and the kidney. In addition, a protective effect against damaging effects of solar radiation or radiation in general is incurred and damage induced by chemical and biological factors is attenuated.

The compounds provided herein may be used to treat a subject, preferably a mammal, more preferably a human, having a condition associated with normally or abnormally proliferating cells. Such conditions associated with abnormally proliferating cells may comprise a pathophysiological condition, such as, but not limited, to a malignant or benign tumor, or a skin disorder or a defect in cellular differentiation, that is, a condition associated with undifferentiated or poorly differentiated cells. Administration of these compounds or pharmaceutical compositions thereof is effective to inhibit abnormal cell proliferation and/or to induce cell differentiation.

Also, the compounds provided herein may be used to treat an autoimmune disease or inflammatory processes caused by the action of NfkB against proliferating cells or immune cells. For example the autoimmune disease or inflammatory process is scleroderma or morphea, keloid or fibromatosis, rheumatoid arthritis, multiple sclerosis, inflammatory bowel diseases, interstitial cystitis, diabetes, obesity atherosclerosis, vasculities, or gout. In general, these compounds or pharmaceutical compositions thereof are effective to inhibit NFκB. NFκB serves as a master regulator of immune processes. Stimulation of NFκB stimulates production of proinflammatory cytokines or mediators, as well as increased expression of proinflammatory molecules on the cell surface. NFκB also is a modifier of cell viability, apoptosis and differentiation. Thus, it is contemplated that inhibition of NFκB may have applications in non-neoplastic diseases, immunology, prevention, and cosmetics.

In addition, the compounds provided herein may be used for cosmetic purposes with both visual and non-visual appealing results. Appealing visual results are healthy, young-looking and esthetically and/or sexually appealing skin and hair with proper coloration and texture and diminution of visible defects. Non-visual appeal refers to an effect on secretory functions of skin adnexal structures and possible pheromone release and the effects against aging of internal organs. Thus, the compounds provided herein may be effective as prophylactic compounds and as promoters of general good health.

The compounds and pharmaceutical compositions thereof may be administered by any method standard in the art and suitable for administration to the subject. Preferably, administration is via a topical composition in a suitable pharmaceutical carrier. Also, the present invention provides that the androsta-5,7-dienes and pregna-5,7-dienes may be administered and subsequently UVB converted in vivo to the corresponding secosterol, tachysterol-like or lumisterol-like conversion product.

Dosage formulations of the compounds of Table 1 and Table 2 and the ether or ester derivatives thereof may comprise conventional non-toxic, physiologically or pharmaceutically acceptable carriers or vehicles suitable for the method of administration. These compounds or pharmaceutical compositions thereof may be administered independently one or more times to achieve, maintain or improve upon a pharmacologic or therapeutic effect. It is well within the skill of an artisan to determine dosage or whether a suitable dosage comprises a single administered dose or multiple administered doses. An appropriate dosage depends on the subject's health, the progression or remission of the disease or disorder, the route of administration and the formulation used.

Thus, the compounds and ether and ester derivatives thereof of Tables 1 and 2 may be efficacious as therapeutics or adjuvant therapeutics for various diseases, disorders or for cosmetic, prophylatic or health maintenance purposes. In addition, these compounds could act as modifiers of action of other biologically active substances. Overall their action would improve the health status either directly or indirectly by modifying the activity of other biologically active agents.

The following examples are given for the purpose of illustrating various embodiments of the invention and are not meant to limit the present invention in any fashion.

### EXAMPLE 1

### Materials and Methods

### UVB Irradiation

A methylene chloride or methanol solution of a compound (1 mg/ml) was subjected to UV irradiation for various times in a quartz cuvette (or glass HPLC insert) using Biorad UV Transilluminator 2000 (Biorad, Hercules, Calif.). Spectral characteristics of the UVB (280-320 nm) source were published previously (10) and it's strength (4.8±0.2 mW cm⁻²) was routinely measured with digital UVB Meter Model 6.0 (Solartech Inc., Harrison Tw p, MI). Irradiation was followed by 14 hours incubation at room temperature or 37° C. and selected products were purified by RP-HPLC chromatography. The major products were identified on the basis of their retention time and characteristic UV absorption. Initial identification w as confirmed by means of MS and NMR measurements. The quantities of products varied and were predominantly dependent on the UVB radiation dose. Fifteen minutes reaction resulted in 30-35% of pre-D-like, 20% T-like, 10% of substrate and lower quantity of other products.

### Reverse Phase-HPLC (RP-HPLC) Chromatography

RP-HPLC analyses were performed using a Waters HPLC-system equipped with a diode-array detector (Waters Associates, Milford, Mass.). The reaction mixture (2-50 μl) of irradiated 5-7 dienes (50-200 μg) was injected by an autosampler onto an Atlantis C18 column (Waters, Ill.) running (Waters, Ill.) using mobile phase of 30% methanol/water at a flow rate of 1.5 mL/min. Fractions were collected every 15 seconds and were reanalyzed by RP-HPLC. Fraction containing above 95% of pure compound (for 240 nm and 280 nm spectra) were pooled and used for further characterization. Chromatogrphic conditions were optimized to achieve best separation for each product.

### MS/NMR Data Collection

Mass spectra were recorded using a Bruker Esquire-LC/MS Spectrometer equipped with an electrospray ionization (ESI) source. The sample was run in 100% methanol at a sample flow rate of 5.0 μL min⁻¹. Chemical shifts were referenced to 3.31 ppm for proton and 49.15 ppm for carbon from solvent peaks. The HDO peak around 4.8 ppm from solvent was suppressed using pre-saturation method for both one-dimensional proton and two-dimensional NMR measurement.

### Cell Culture

Immortalized human keratinocytes (HaCaT) were cultured in Dulbecco's Modified Eagle Medium supplemented with glucose, L-glutamine, pyridoxine hydrochloride (Cell Grow), 5% fetal bovine serum (Sigma) and 1% penicillin/streptomycin/amphotericin antibiotic solution (Sigma). Human adult epidermal keratinocytes were grown in EpiLife medium with Human Keratinocyte Growth Supplement (HKGS) and gentamycin and amphotericin B solution (Cascade Biologics, Inc., Portland, Oreg.). Melanoma cells: human SK Mel 188 and hamster AbC1 were grown in F10 media (Gibco) supplemented with 5% fetal bovine serum. Prostate cancer cells (PC3) were cultured in Dulbecco's Modified Eagle Medium supplemented with glucose, L-glutamine, pyridoxine hydrochloride (Cell Grow), 5% fetal bovine serum (Sigma) and 1% penicillin/streptomycin/amphotericin antibiotic solution (Sigma). HL-60 human promyelocytic and 0937 promonocytic leukemia cells (10×106), and K562 human chronic myeloid leukemia and MEL mouse erytholeukemia cells (2×106) were cultured in RPMI media containing 10% fetal bovine serum (10 ml per flask).

### DNA Synthesis

HaCaT keratinocytes were plated in 96-well plates at the density of 10,000 cells/well in DMEM (Cellgro, Herndon Va.) containing 5% charcoal treated fetal bovine serum (Hyclone, Logan, Utah), 1% antibiotic solution (PSA, Sigma, St. Louis, Mich.). Next day, media were changed and vehicle (ethanol) or secosteroids added. Cells were incubated with compounds for 48 hours. [³H]-thymidine (Amersham Biosciences, Picataway, N.Y.) was added to the final concentration of 1 μCi/mL medium for last 12 hours of incubation. Media were then discarded, cells detached with trypsin and harvested on a glass fiber filter (Packard, Meriden, Calif.). Radioactivity was measured with a beta counter (Direct Beta-Counter Matrix 9600; Packard).

### Proliferation, Differentiation and Clonogenicity Assay

HaCaT and human adult epidermal keratinocytes were cultured and DNA synthesis experiments were performed as described previously (26). Cells were plated in 6-well plates at a density of 20 cells/cm² in DMEM (Cellgro, Herndon Va.) containing 5% charcoal-treated fetal bovine serum (Hyclone, Logan, Utah), 1% antibiotic solution (PSA, Sigma, St. Louis, Mich.) and vehicle or secosteroids. Cells were incubated in 37° C. for 10 days with media being changed every 72 hr. Cells were fixed with 4% paraformaldehyde in PBS overnight, stained with 0.5% crystal violet in PBS for 15 min, rinsed and air-dried. The number and size of the colonies were measured using an ARTEK counter 880 (Dynex Technologies Inc., Chantilly, Va.). Colony forming units were calculated as follows: number of colonies (size>0.5 mm) was divided by the number of cells plated and multiplied by 100.

HL-60 human promyelocytic and U937 promonocytic leukemia cells were treated with the drugs at 0.1 μM or vehicle (negative control or 12-O-tetradecanoylphorbol-13-acetate (TPA) (positive control). Media were changed every 72 h and test substances added fresh every day. Differentiation toward monocytes-like morphology and NBT-reduction has been assessed after 5 and 7 days. Cells (2×106) were washed with PBS four times and resuspended in 2004 of NBT solution (4 mg/mL) in water. After the addition of 200 ul of TPA solution (2 ug/ml) in PBS cells were incubated at 37° C. for 60 min in 24-well plates. Cell differentiation was assessed by intracellular blue formazan deposits. The NBT positive and negative cells were scored under light microscopy examination (40×) with a minimum of 200 cells scored.

For spectrophotometric analysis the cells were washed twice with buffer containing cold bovine serum albumin solution (17 mg/mL BSA, 137 mmol/L, NaCl, 5 mmol/L, KCl, 0.8 mmol/L, MgSO4, 10 nmol/L, HEPES, pH7.4) to remove unreacted NBT, and the insoluble formazan deposits in the resulting pellet were solubilized in 1 mL of a mixture containing 90% DMSO, 0.1% SDS and 0.01 mmol/L NaOH by vigorous vortexing. The samples were centrifuged 5 min at 1500 g to remove the cellular debris, and then the absorbance of supernatants was measured at 715 nm. Data are expressed as change in A715/106 cells.

### Flow Cytometry for DNA Content Analysis

DNA content analysis was performed with a FACS Calibur flow cytometer as described previously (26). HaCaT cells were treated with 20,23-dihydroxycholecalciferol and 1α,25-dihydroxycholecalciferol at different concentrations ranging from 0.1 nM-10 nM for 24 h. After treatment cells were harvested by trypsinisation, washed in PBS, fixed in 70% cold ethanol and stained with propidium iodine (Sigma). For analysis of involucrin expression, after treatment cells were fixed with cold 2% paraformaldehyde in PBS for 1 hour. Pellets (200,000 cells per sample) were washed in PBS and resuspended in 100 μL of permeabilizing solution containing saponin 0.25%, 0.1% BSA, 0.1% NaN₃ in PBS, and primary antibody against human involucrin (0.2 μg, amount of the antibody added was set after preliminary titration experiment, Novocastra Laboratories Ltd, Newcastle). Cells stained with isotype control antibody (IgG1, Caltag Laboratories, Burlingame, Calif.) were used as controls.

After 12 hours of incubation, cells were washed twice with PBS and resuspended in 100 μL of permeabilizing solution containing sheep anti-mouse secondary FITC-conjugated antibody (1:50, Novocastra Laboratories Ltd, Newcastle upon Tyme, United Kingdom). After 3 hours cells were washed with PBS and then resuspended in 400 μL of PBS. Samples were read with a FACS Calibur flow cytometer.

The FL-1 signal (collected from 10,000 events in side scatter/forward scatter window after debris exclusion) was recorded. Forward (relative to cell size) and side (relative to cell granularity) scatter histograms were generated and mean signal intensity was recorded. FL-1 signal values are presented as dMFI (difference between mean fluorescence intensity of sample stained with specific and isotype control antibody). Scatter signal values are presented as MSI (mean signal intensity). Signal intensities were analyzed with Cell Quest (BD Biosciences, San Diego, Calif.) and graphical representations of the FL-1 signal were prepared with WinMdi 2.8 (shareware from Joseph Trotter, The Scripps Research Institute, San Diego, Calif.).

### Microscopic Analysis of Involucrin Expression

Cells were seeded in 6-well Lab-Tek II chamber slides (Nalge Nunc, Inc., Naperville, Ill.). Cells were pre-incubated in Epilife medium with HKGS overnight and then stimulated with 20-hydroxycholecalciferol in Epilife medium with HKGS for 24 hours and then fixed with 4% paraformaldehyde in PBS for 10 minutes. The cells were permeabilized with 0.2% Triton-X 100 in PBS for 5 minutes and blocked with 1% bovine serum albumine (BSA; in PBS) for 30 minutes. The cells were incubated consecutively with mouse anti-human involucrin antibody (Novocastra, Newcastle upon Tyne, UK) for 3 hours, anti-mouse-fluorescein isothiocyanate (FITC) conjugate (Novocastra, Newcastle upon Tyne, UK) for 1 hour in buffer containing 1% BSA in PBS. The slides were extensively washed with PBS between stainings and mounted with Vectashield mounting medium with propidium iodide (Vector Laboratories, Burlingame, Calif.). Slides not incubated with primary antibody were used as background controls. Slides were viewed with NIKON Eclipse TE300 microscope (Melville, N.Y.).

### Real-Time RT PCR for Cytokeratin 14, Involucrin, CYP27A1 and CYP27B1

RNA was extracted using an Absolutely RNA RT-PCR Miniprep Kit (Stratagene, La Jolla, Calif.). Real time PCR and reverse transcription products were purchased from Applied Biosystems, Foster City, Calif. Reverse transcription was performed using Taqman® Reverse Transcription Reagents. The following PCR products were used: cytokeratin 14: Hs00265033_m1, involucrin: Hs00846307_s1, CYP27A1: Hs00168003_m1, CYP27B1: Hs00168017_m1, 18SrRNA: Hs99999901_s1. The reaction was performed with Taqman® Universal PCR Master Mix; data were collected on an ABI Prism 7700 and analyzed on Sequence Detector 1.9.1. Specific gene amounts were related to 18SrRNA by comparative C_{T} method.

### Real-Time RT PCR for CYP24

RNA was extracted as above. Reverse transcription was performed with Transcriptor First Strand cDNA Synthesis Kit (Roche, Nutley, N.J.). The primers (right: 5′-GCA GCT CGA CTG GAG TGA C-3′ (SEQ ID NO: 1) and left: 5′-CAT CAT GGC CAT CAA AAC AAT-3′ (SEQ ID NO: 2)) and probe (cat. no. 04689135001) were designed with Universal Probe Library (Roche, Nutley, N.J.). Real-time PCR was performed using TaqMan PCR Master Mix at 50° C. for 2 min, 95° C. for 10 min and then 50 cycles (95° C. for 15 sec, 60° C. for 1 min). The data was collected on a Roche Light Cycler 480. The amounts of CYP24 were normalized using cyclophilin D as a housekeeping gene with comparative C_{T} method.

### CYP24-Luc Transfection

The CYP24-Luc construct was a generous gift from Dr Tai Cheng (Boston University Medical Campus, Core Lab Director). It was originally developed by Vaisanen et al. (27). The details of the pLuc construct have been described previously (28-29). Normal epidermal keratinocytes were transfected using Lipofectamine Plus (Invitrogen, Carlsbad, Calif.) in Epilife medium with firefly luciferase reporter gene plasmid and with phRL-TK (expresses Renilla luciferase and serves as normalization control; Promega, Madison, Wis.). After transfection, cells were incubated for 24 hours in EpiLife medium with HKGS. Cells were then transferred to fresh media containing the compounds to be tested or vehicle (ethanol) and incubated for 24 hours. The firefly luciferase and Renilla luciferase signals were recorded with a TD-20/20 luminometer (Turner Designs, Sunnyvale, Calif.); background luminescence was subtracted and the resulting promoter-specific firefly signal was divided by the Renilla signal (proportional to the number of transfected cells). The values obtained were divided by the mean of control (untreated) cells.

### Electrophoretic Mobility Shift Assay for VDR Activation

HaCaT keratinocytes were treated with 20,23-dihydroxycholecalciferol compound at the concentration 1 nM, 10 nM and 100 nM and EtOH as control for 24 h. The cells were collected with trypsin/EDTA, washed with 1× PBS and resuspended in 1 ml of 0.2% Triton-X 100 in STM buffer containing 20 mM Tris-Cl, 250 mM sucrose, 1.1 mM MgCl2. Cell suspension was vortexed and incubated on ice for 10 minutes followed by 15 second centrifugation at 4° C. Whole step was repeated twice. Cell pellets were than resuspended in 1 ml STM buffer and centrifuged for 15 seconds. This step was also repeated for two times. The nuclear pellet was resuspended in 30 μl nuclear extraction buffer containing 0.4M KCl, 5 mM 2-Mercaptoethanol and protease inhibitors cocktail (1:100 dilution, Sigma) in STM buffer and incubated on ice for 30 minutes with shaking and than centrifuged at 14,000 g for 20 minutes at 4° C. The supernatant was quantified using Bradford protein assay kit.

Electrophoretic mobility shift assay (EMSA) was done using the Odyssey Infrared

Imaging System (LI-COR, Inc,. Lincoln, NE). The synthetic IRDye-labeled oligonucleotide (LI-COR) used for the DNA mobility shift assay contained the wild-type VDRE sequence sp1 promoter and part of INVOLUCRIN sequence contained the sequence: 5′-GCG GGA GGC AGA TCT GGC AGA TAC TGA-3′ (SEQ ID NO: 3). Oligo was end labeled with infrared dye 700. Unlabeled oligo contained the same sequence. The DNA binding reaction was set up using 2.5 μg of the nuclear extract mixed with oligonucleotide and gel shift binding buffer consisting of: 2.5 mmol/L DTT, 0.25% Tween-20 and 0.25 mg/ml poly(dI):poly(dC) according to the LI-COR protocol. The reaction was incubated at room temperature in the dark for 30 minutes. For NF-κB activation p65 antibody was added to the 1 sample and incubated for the 30 min. Orange loading dye (2 μl of 10×) was added to each sample and loaded on the prerun 5% polyacrylamide gel and ran at 80V for 1 hour. The gel was scanned using Odyssey Infrared Imaging System.

### Electrophoretic Mobility Shift Assay for NF-κB Activation

HaCaT keratinocytes were treated with 20,23-dihydroxycholecalciferol for indicated time: 0 h, 30 min, 1 h, 4 h, 16 h and 24 h, at the concentration of 10⁻⁷ [M]. The cells were prepared and the EMSA assay was performed as for VDRE activation except that p65 antibody was added to the 1 sample and incubated for the 30 min prior to adding the orange loading dye and that the gel was run at 80V for 1.5 hours.

### Western Blot

HaCaT keratinocytes were treated with 20,23-dihydroxycholecalciferol for indicated time: 0 h, 30 min, 1 h, 4 h, 16 h and 24 h, at the concentration of 10⁻⁷ [M]. Cells were lysed and whole cell extract has been prepared. The equal amount of proteins calculates using Bradford method has been subjected to electrophoresis in SDS-PAGE 7-15% gel and transferred to a PVDF membrane (Millipore). The primary antibodies used were the rabbit polyclonal antibodies of anti-IκB-α (Santa Cruz), 1:250 dilution, anti-p65 (Santa Cruz) 1:500 dilution and anti-β actin-peroxidase (Sigma) 1:1000 dilution. Secondary antibody used was anti rabbit-HRP (Santa Cruz) 1:7,000 dilution.

VDRE-Luc and siRNA Transfection

HaCaT keratinocytes were transfected with VDRE-Luc (gift from Dr. Thai Chen (27) and with scrambled or VDR siRNA (Dharmacon, Inc., Lafayette, Mo., on-Target plus smart pool human VDR L-003448-00, on-Target plus siControl non-targeting pool D-001810-10-05) using Lipofectamine Plus (Invitrogen, Carlsbad, Calif.) in DMEM medium. PhRL-TK (expresses Renilla luciferase) served as a normalization control; Promega, Madison, Wis.). After transfection, cells were incubated for 24 hours in DMEM with 5% FBS. Cells were then transferred to fresh media containing the compounds to be tested or vehicle (ethanol) and incubated for 24 hours. Levels of VDR and beta-actin 24 h after transfection were assessed with Western blot (VDR(D-6) antibody, sc-13133, 1:400, Santa Cruz, Inc., Santa Cruz, Calif.) performed as described previously (30.).

### [³H]-Thymidine Incorporation

To measure DNA synthesis, keratinocytes, melanoma or prostate cancer cells were plated in 96-well plates. After overnight incubation tested compounds were added to the medium to achieve final concentrations 10⁻⁷-10⁻¹⁰ [M]. 100 μl of medium per well containing vitamin D3 compound was added to the cells. After 36 hours of incubation [3H]-thymidine (specific activity 88.0 Ci/mmol; Amersham Biosciences, Picataway, N.Y., USA) was added at 1 μCi/mL medium. After 12 hours, media were discarded, cells detached with trypsin and harvested on a glass fiber filter (Packard, Meriden, Calif., USA). 3H-radioactivity was measured with a beta counter (Direct Beta-Counter Matrix 9600; Packard).

### Cell Viability Assay (MTT Assay)

MTT test were performed. Briefly, the cells were seeded in 96-well plate. Following 24, 48, 72 or 96 h incubation, MTT (5 mg/mL in PBS, Promega, Madison, Wis.) was added and the plates were incubated at 37° C. in 5% CO2 for 4 h. Subsequently, medium was discarded, acid isopropanol was added, plates were incubated for 30 min with continuous shaking and absorbance was measured at 570 nm with a plate reader (BIO-RAD Laboratories, Hercules, Calif.).

### Cell Viability Estimated by Sulforhodamine B (SRB) Assay

The cells were seeded in 96-well plate in F10 medium. Following 48 to 96 h incubation cells were fixed with 10% trichloroacetic acid, washed and then incubated with 0.04% sulforhodamine B (in 1% acetic acid) for 30 min. Following second wash with 1% acetic acid, dye incorporated into the cells was solubilized in 10 mM Tris by shaking for 30 min and the absorbance was measured at 570 nm.

### Soft Agar Colony Formation Assay

Growth and survival of prostate cancer cells (PC3) and melanoma cells SKMel 188 and AbC1 was determined by following their ability to form colonies in soft agar. Cells growing in monolayer culture were trypsinized and resuspended (1,000 cells/well) in 0.25 ml medium containing 0.4% agarose and 5% charcoal stripped serum (HyClone).

Cell suspensions were added to 0.8% agar layer in 24 well plates. Test compounds at different concentrations ranging from 0.1 nM-100 nM was added in between two layers of agarose and in quadruplicates. Cells examined immediately after plating showed only single colonies. After two weeks agar colonies were scored and stained overnight with 0.5 mg/mI MTT reagent (Promega). Colonies were counted under the microscope and number of units was calculated as number of colonies formed divided by the number of cells seeded×100.

### Transfection and Reporter Assay

Construct CYP24-Luc and VDRE-Luc constructs were a generous gift from Dr. Tai Cheng, Ph.D. (Boston University Medical Campus, Core Lab Director). It was originally developed by Vaisanen et al. (27). PLuc and NFKB-Luc construct was described previously (28-29). huVDR construct was a generous gift from Dr. D. Bikle. HaCaT keratinocytes were transfected with NFκB-Luc, CYP-24-Luc, VDRE-Luc and hVDR constructs using Lipofectamine Plus (Invitrogen, Carlsbad, Calif.) in DMEM medium with firefly luciferase reporter gene plasmid and with phRL-TK.

HaCaT keratinocytes were transfected with NFKB-Luc construct using Lipofectamine Plus (Invitrogen, Carlsbad, Calif.) in DMEM medium with firefly luciferase reporter gene plasmid and with phRL-TK (expresses Renilla luciferase and serves as normalization control; Promega, Madison, Wis.). After transfection cells were incubated for 24 h in complete medium, and then after change to fresh media treated with drug or vehicle (ethanol) at the concentration of 10 nM for 30 minutes, 1 h, 4 h, 16 h and 24 h. The firefly luciferase and Renilla luciferase signals were recorded with a TD-20/20 luminometer (Turner Designs, Sunnyvale, Calif.); background luminescence was subtracted and the resulting promoter specific firefly signal was divided by the Renilla signal (proportional to the number of transfected cells). The values obtained were divided by the mean of control (untreated) cells.

### Assessment of Erythroid Differentiation

K562 human chronic myeloid leukemia (erythroleukemia) and MEL mouse erytholeukemia cells were cultured in RPMI 1640 containing 10% FBS and treated with the drugs at concentrations 0.1 μM. A 0.1% ethanol (EtOH) or 2% DMSO were used as negative or positive control, respectively to estimate the vehicle effect and the ability of the cells to differentiate. Media were exchanged every 72 h and drugs added every day. Growth of cultures was estimated by counting number of viable cells (trypan blue negative cells) as described previously.

To estimate erythroid differentiation (production of hemoglobin), first the number of benzidine positive cells was evaluated after 2 and 7 days in culture. Cells were centrifuged and washed four times with PBS and resuspended in 1 mL of fresh PBS. For hemoglobin determination, a benzidine staining solution was freshly prepared by mixing one part of 30% hydrogen peroxide, one part of base stock solution of 3% benzidine in 90% acetic acid, and 5 parts of water. The solution was diluted 1:10 with the cell suspension and aliquoted in 4 wells in 24-well plate (250 μl each). After 10 min of incubation at RT benzidine-positive cells were counted under the microscope with a minimum of 200 cells scored.

Second, to define spectrophotometrically relative content of hemoglobin, equal number of cells (7×106) were washed with cold PBS and lysed for 20 min in 100 μL of lysis buffer (0.2% Triton X-100 in 100 nM potassium phosphate buffer, pH 7.8). The lysates were centrifuged for 15 min at 1500 r.p.m. and 100 μL of the supernatant was incubated with 2 mL of benzidine solution (5 mg/mL in glacial acetic acid) and 2 ml 30% H2O2 for 10 min increases in comparison with the level of hemoglobin in mock-induced K562 or MEL cells.

### Statistical Analysis

Data were analyzed with GraphPad Prism Version 4.0 (GraphPad Software Inc., San Diego, Calif., USA) using t test. Differences were considered significant when p<0.05. The data are presented as means±SE.

### EXAMPLE 2

### Chemical Synthesis Methods

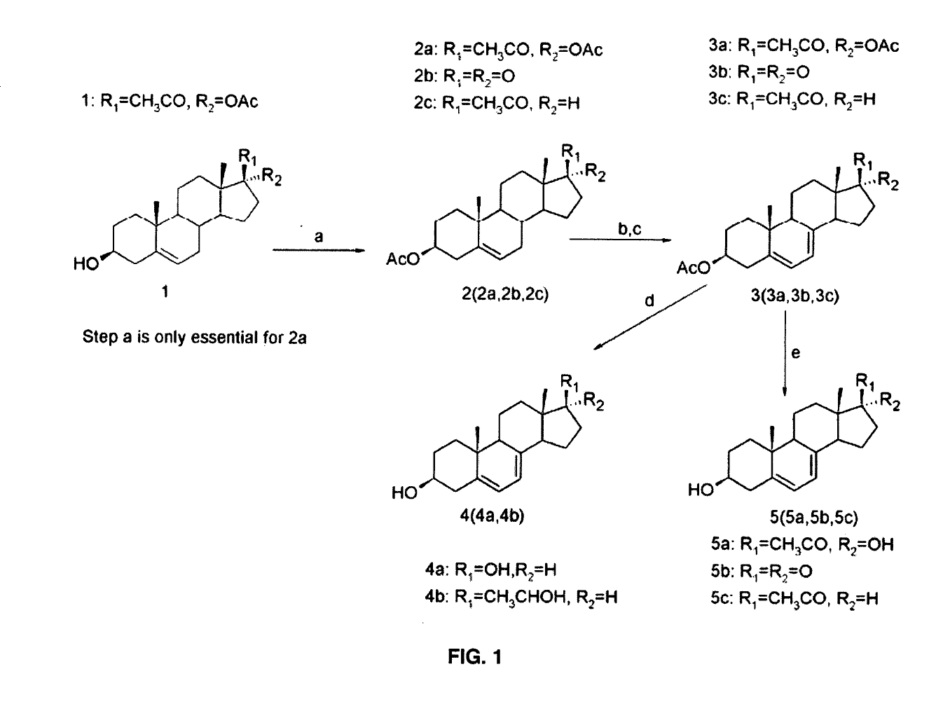

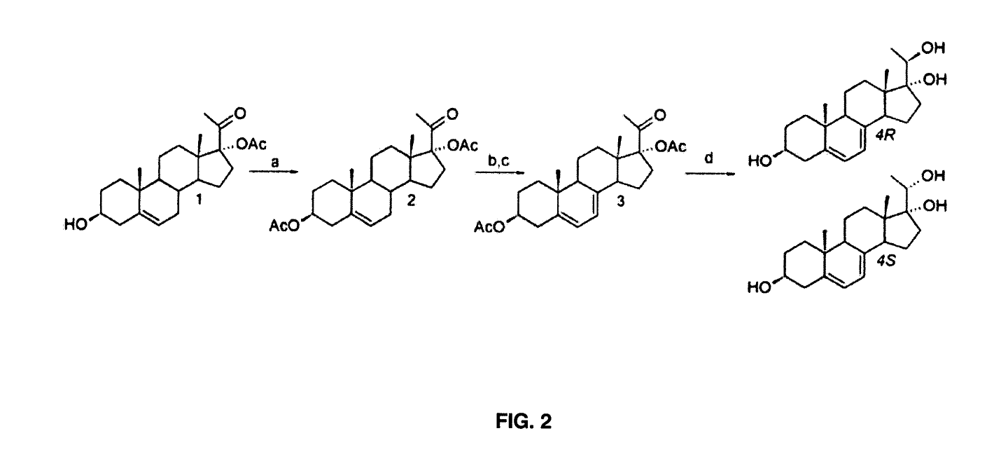

The sequence of the synthesis of compounds 4 (4a, 4b) and compounds 5 (5a, 5b, 5c) is shown in FIG. 1 and of 4 (4R and 4S) is shown in FIG. 2.

### General Synthesis of Compounds 4 (4a, 4b) and Compounds 5 (5a, 5b, 5c)

The acetylation of 17α-acetoxypregnenolone 1 was carried out follow ing the known procedure (31). Yield: 95%. ¹H NMR (500 MHz, CDCl₃) for compound 2a: δ 5.39 (d, J=5 Hz, 1H), 4.58-4.64 (m, 1H), 2.92-2.96 (m, 1H), 2.30-2.36 (m, 2H), 2.12 (s, 3H), 2.04 (s, 3H), 2.05 (s, 3H), 1.98-2.02 (m, 2H), 1.86-1.90 (m, 2H), 1.46-1.80 (m, 9H), 1.26-1.32 (m, 1H), 1.14-1.18 (m, 1H), 1.06-1.08 (m, 1H), 1.03 (s, 3H), 0.64 (s, 3H). ESI-MS: calculated for C₂₅H₃₆O₅, 416.26, found 439.3 [M+Na]⁺.

Compounds 3 (3a, 3b, 3c) were synthesized according to a know n procedure (12). Yield: 40-50%. ¹H NMR (500 l MHz, CDCl₃) for compound 3a: δ 5.57-5.59 (dd, J=10 Hz, 3.0 Hz, 1H), 5.44-5.46 (m, 1H), 4.68-4.74 (m, 1H), 2.96-2.90 (m, 1H), 2.59-2.63 (m, 1H), 2.49-2.54 (m, 1H), 2.36 (t, J=15 Hz, 1H), 2.11 (s, 3H), 2.08 (s, 3H), 2.05 (s, 3H), 2.02-2.04 (m, 1H), 1.82-1.94 (m, 4H), 1.56-1.73 (m, 6H), 1.38 (dt, J=15 Hz, 5 Hz, 1H), 0.95 (s, 3H), 0.57 (s, 3H). ESI-MS: calculated for C₂₅H₃₄O₅, 414.24, found 437.3 [M+Na]⁺.

¹H NMR (300 MHz, CDCl₃) for compound 3b: δ 5.51-5.53 (dd, J=10 Hz, 3.6 Hz, 2H), 4.53-4.55 (m, 1H), 1.34-2.60 (m, 16H), 2.08 (s, 3H), 0.99 (s, 3H), 0.83 (s, 3H). ESI-MS: calculated for C₂₁H₂₈O₃, 328.20, found 351.3 [M+Na]⁺.

¹H NMR (500 MHz, CDCl₃) for compound 3c: δ 5.59-5.61 (dd, J=12 Hz, 4.0 Hz, 1H), 5.44-5.46 (m, 1H), 4.70-4.77 (m, 1H), 2.64-2.68 (t, J=10 Hz, 1H), 2.52-2.55 (m, 1H), 2.36-2.42 (m, 1H), 2.22-2.26 (m, 1H), 2.17 (s, 3H), 2.14-2.16 (m, 1H), 2.06 (s, 3H), 1.72-1.96 (m, 8H), 1.52-1.62 (m, 3H), 1.37-1.43 (dt, J=30 Hz, 5 Hz, 1H), 0.97 (s, 3H), 0.60 (s, 3H). ESI-MS: calculated for C₂₃H₃₂O₃, 356.24, found 379.3 [M+Na]⁺.

Compounds androsta-5,7-diene-3β,17α-diol 4a and pregna-5,7-diene-3β,20-diol 4b were synthesized from the precursor 2 according as described (32) where the deprotection reaction was carried on simultaneously with reduction of the carbonyl group. Interestingly, only synthesis of 9β,10α-androsta-5,7-diene-3β,17α-diol 4a resulted in a mixture of 4,6- and 5,7-dienes, where the 5,7-diene constituted 95% of the mixture after purification. The 4,6-diene was subsequently removed by silica gel-AgNO₃ chromatography (33). Compounds 3β,17α-dihydroxy-9β,10α-pregna-5,7-dien-20-one 5a, 3β-hydroxypregna-5,7-dien-17-one 5b and 3β-hydroxypregna-5,7-dien-20-one or 7-dehydropregnenolone 5c also were synthesized from the precursor 2, how ever only a deprotection reaction was carried out on intermediate compound 3.

Yield: 45-55%. ¹H NMR (500 MHz, CD₃OD) for compound 5L 4a: δ 5.57 (dd, J=10 Hz, 6 Hz, 1H), 5.38-5.41 (m, 1H), 3.70-3.73 (m, 1H), 3.50-56 (m, 1H), 2.42-2.45 (m, 1H), 2.26 (t, J=10 Hz, 1H), 2.07-2.13 (m, 1H), 1.86-2.00 (m, 6H), 1.68-1.76 (m, 3H), 1.46-1.60 (m, 4H), 1.32 (dt, J=30 Hz, 6 Hz, 1H), 1.20 (dt, J=25 Hz, 10 Hz, 1H), 0.98 (s, 3H), 0.70 (s, 3H). ESI-MS: calculated for C₁₉H₂₈O₂, 288.21, found 311.3 [M+Na]⁺. ¹H NMR (500 MHz, CD₃OD) for compound 4b: δ 5.57 (dd, J=13.5 Hz, 4 Hz, 1H), 5.39-5.41 (m, 1H), 3.60-3.77 (m, 2H), 2.44-2.50 (dq, J=32 Hz, 12.5 Hz, 4 Hz, 1H), 2.29 (t, J=19.5 Hz, 3.0 Hz, 1H), 2.15-2.21 (m, 1H), 1.22-2.08 (m, 16H), 1.15-1.17 (d, J=10 Hz, 3H), 0.96 (s, 3H), 0.71 (s, 3H). ESI-MS: calculated for C₂₁H₃₂O₂, 332.24, found 355.3.

### General Synthesis of 4: 20R and 20S Epimers of Pregna-5,7-diene-3β,17α,20-triol (4R and 4S)

The synthesis of pregna-5,7-diene-3β,17α,20-triols (4R and 4S) was carried out from 17α-acetylated 5-en precursor 1 by acetylation, bromination-dehydrobromination and reduction, following the known procedure (32). Deprotection was performed simultaneously with reduction of the carbonyl group. The procedure resulted in formation of two diastereomers: 4R and 4S (20R and 20S) with a ratio of 50:50. The mixture was effectively separated by reverse phase HPLC (RP-HPLC). The presence of 4,6-dienes was not detected. Reaction resulted in a crude, white, solid mixture of compounds 4R and 4S: 40-50% yield. The mixture was subjected to flash chromatography (column eluted with hexane-ethyl acetate 20:1, 10:1, 5:1, 1:1 in order), but only the isomer with a faster retention time (Peak 1, 4R), was purified in this manner. The separation of the second isomer (Peak 2, 4S) was accomplished using RP-HPLC.

¹H NMR (500 MHz, CD₃OD) for compound 4S: δ 5.54 (dd, J=7.5 Hz, 3.2 Hz, 1H), 5.38-5.40 (m, 1H), 3.94 (q, J=18 Hz, 1H), 3.47-3.55 (m, 1H), 2.57 (t, J=10 Hz, 1H), 2.38-2.43 (m, 1H), 2.2-2.28 (m, 1H), 1.88-1.98 (m, 3H), 1.62-1.88 (m, 8H), 1.42-1.58 (m, 3H), 1.26-1.34 (m, 2H), 1.15 (d, J=6 Hz, 3H), 0.96 (s, 3H), 0.77 (s, 3H). ESI-MS: calculated for C₂₁H₃₂O₃, 332.24, found 355.3 [M+Na]⁺; 4R: δ 5.54 (dd, J=8.3 Hz, 2.5 Hz, 1H), 5.41-5.44 (m, 1H), 3.79 (q, J=18 Hz, 1H), 3.47-3.56 (m, 1H), 2.60 (t, J=10 Hz, 1H), 2.38-2.43 (m, 1H), 2.20-2.28 (m, 1H), 2.07-2.14 (m, 1H), 1.89-1.97 (m, 2H), 1.62-1.89 (m, 8H), 1.42-1.53 (m, 2H), 1.26-1.34 (m, 2H), 1.12 (d, J=6 Hz, 3H), 0.95 (s, 3H), 0.7 (s, 3H). ESI-MS: calculated for C₂₁H₃₂O₃, 332.24, found 355.2 [M+Na]⁺.

UV and MS data of synthesized pregna-5,7-diene-3β,17α,20-triols and derivatives are summarized in Table 3 and the detailed NMR data are presented on Table 4. NMR chemical shifts for 4R and 4S are in agreement with those presented previously (32), with small differences related to a solvent effect due to the use of CD₃OD instead of CDCl₃ for NMR experiments.

TABLE 3

| | Parent | | | Predicted | |
|---|---|---|---|---|---|
| No | cmpd | Structure type | UV max (nm) | MW | MS+ |
| 4R | 1 | 5,7-diene | 261, 270, 281, 290 | 332.25 | 355.25 [M + Na]+ |
| 4R1 | 4R | preD-like | 260 | 332.25 | ND |
| 4R-D | 4R | D-like | 265 | 332.25 | 355.25 [M + Na]+ |
| 4R-L | 4R | L-like | 262, 271, 281 | 332.25 | 355.25 [M + Na]+ |
| 4R-T | 4R | T-like | 270, 279, 291 | 332.25 | 355.25 [M + Na]+ |
| 4R-iT | 4R | isoT-like (oxide) | 240, 248, 257 | 364.25 | 387.15 [M + Na]+ |
| 4R7 | 4R | 5,7,9(11) triene | 312, 323, 340 | 330.22 | ND |
| 4S | 1 | 5,7-diene | 263, 271, 282, 292 | 332.25 | 355.25 [M + Na]+ |
| 4S1 | 4S | preD-like | 260 | 332.25 | ND |
| 4S-D | 4S | D-like | 265 | 332.25 | 355.25 [M + Na]+ |
| 4S-L | 4S | L-like | 262, 271, 282 | 332.25 | 355.25 [M + Na]+ |
| 4S-T | 4S | T-like | 272, 282, 291 | 332.25 | 355.25 [M + Na]+ ND |
| 4S-iT1 | 4S | isoT-like | 241, 248, 257 | 332.25 | 355.25 [M + Na]+ |
| 4S-iT2 | 4S | isoT-like (oxide) | 241, 248, 257 | 332.25 | 387.15 [M + Na]+ |
| 4S7 | 4S | 5,7,9(11) triene | 312, 323, 340 | 330.22 | ND |

Bold-purified and characterized (NMR), italics-characterized by UV spectra, ND—not determined

TABLE 4

| | 4R | 4S | 4R-D | 4S-D | 4R-L | 4S-L | 5S |
|---|---|---|---|---|---|---|---|
| 1 CH₂ | α1.31 | α 1.29 | α 2.12 | α2.12 | α 1.31 | α 1.31 | 1.48 |
| | β 1.90 | β1.90 | β 2.42 | β 2.41 | β 1.90 | β 1.90 | 1.70 |
| 2 CH₂ | α 1.92 | α 1.93 | α 1.97 | α1.97 | α 1.92 | α 1.92 | 1.89 |
| | β 1.45 | β 1.54 | β 1.68 | β 1.68 | β 1.54 | β 1.54 | 1.68 |
| 3 CH | 3.51 | 3.51 | 3.76 | 3.76 | 4.03 | 4.03 | 3.47 |
| 4 CH₂ | α 2.41 | α 2.40 | α 2.55 | α 2.54 | α 2.41 | α 2.44 | 2.34 |
| | β 2.23 | β 2.23 | β 2.19 | β 2.19 | β 2.29 | β 2.35 | 2.42 |
| 6 CH | 5.54 | 5.54 | 6.23 | 6.24 | 5.59 | 5.54 | 5.65 |
| 7 CH | 5.42 | 5.39 | 6.09 | 6.09 | 5.47 | 5.39 | 5.40 |
| 9 CH | 2.11 | 2.02 | 2.85 | 2.85 | 2.02 | 2.02 | |
| | | | 1.54 | 1.56 | | | |
| 11 CH₂ | α 1.67 | α 1.6-1.8 | α 1.77 | α 1.77 | α 1.7-1.8 | α 1.7-1.8 | 5.61 |
| | β 1.75 | β 1.6-1.8 | β 1.77 | β 1.77 | β 1.7-1.8 | β 1.7-1.8 | |
| 12 CH₂ | α 1.49 | α 1.49 | α 1.54 | α 1.56 | α 1.49 | α 1.49 | 2.61 |
| | β 1.80 | β 2.12 | β 2.05 | β 2.04 | β 2.18 | β 2.18 | 2.14 |
| 14 CH | 2.62 | 2.57 | 2.12 | 2.12 | 2.05 | 2.05 | 2.78 |
| 15 CH₂ | α 1.83 | α 1.84 | α 1.54 | α 1.61 | α 1.82 | α1.82 | 1.85 |
| | β 1.63 | β 1.56 | β 2.53 | β 1.61 | β 1.51 | β 1.51 | 1.50 |
| 16 CH₂ | α 1.56 | α 1.7-1.8? | α 1.87 | α 1.7 | α 1.76 | α 1.76 | 1.60 |
| | β 1.74 | β 1.9? | β 2.7 | β 2.66 | β 2.22 | β 2.22 | 1.75 |
| 18 CH₃ | 0.7 | 0.77 | 0.6 | 0.67 | 0.76 | 0.76 | 0.67 |
| 19 CH₃ | 0.94 | 0.95 | β 4.76 | β 4.74 | 0.67 | 0.75 | 1.24 |
| | | | β 5.05 | β 5.05 | | | |
| 20 C | 3.78 CH | 3.94 CH | 3.76 | 3.61 | 3.72 CH | 3.65 CH | 3.9 |
| 21 CH₃ | 1.19 | 1.15 | 1.18 | 1.14 | 1.14 | 1.15 | 1.16 |

Italics - not fully determined presumably similar to parental compound

Compounds 5: 3β,17α-dihydroxypregna-5,7-dien-20-one 5a, 3β-hydroxyandrosta-5,7-dien-17-one 5b and 3β-hydroxypregna-5,7-dien-20-one 5c were synthesized according to a known procedure (32). The synthesis of 5c from pregnenolone acetate (2c) was initially carried out by a bromination/dehydrobromination method, followed by hydrolysis of the acetyl group at C-3 (34). However, this standard procedure resulted in a mixture of 95% pregna-4,6-dienes and only 5% 5,7-dienes, i.e., 3β-hydroxypregna-4,6-dien-20-one 6c and 3β-hydroxypregna-5,7-dien-20-one 5c. This mixture of isomers was separated by silica gel-AgNO₃ chromatography (33) and products were characterized by their distinctly different UV (λₘₐₓ 233, 238, 248 nm for 4, 6-diene and λₘₐₓ 262, 272, 283, 294 for 5,7-diene) and NMR spectra. To improve the yield of the desired 5,7-diene, the alternative method for the synthesis of 33-hydroxypregna-5,7-dien-20-one 5c and the other 5,7-dienes 5a-5b were adopted (32-33).

Yield: 50-60%. ¹H NMR (300 l MHz, CDCl₃) for compound 5a: α 5.59-5.60 (dd, J=10 Hz, 5.0 Hz, 1H), 5.46-5.47 (m, 1H), 3.65-3.67 (m, 1H), 2.62-2.75 (m, 2H), 2.48-2.52 (m, 1H), 2.29 (s, 3H), 1.26-2.25 (m, 15H), 0.96 (s, 3H), 0.71 (s, 3H). ESI-MS: calculated for C₂₁H₃₀O₃, 330.22, found 353.3 [M+Na]⁺.

¹H NMR (500 MHz, CDCl₃) for compound 5b: δ 5.63-5.64 (dd, J=8.0 Hz, 3.0 Hz, 1H), 5.57-5.59 (m, 1H), 3.65-3.70 (m, 1H), 2.50-2.58 (m, 2H), 2.32 (t, J=15 Hz, 25 Hz, 1H), 2.18-2.25 (m, 2H), 2.05-2.14(m, 2H), 1.90-1.97 (m, 3H), 1.73-1.82 (m, 3H), 1.52-1.54 (m, 1H), 1.28-1.41 (m, 3H), 0.98 (s, 3H), 0.84 (s, 3H). ESI-MS: calculated for C₁₉H₂₆O₂, 286.2, found 309.3 [M+Na]⁺.

¹H NMR (500 MHz, DMSO) for compound 5c: δ 5.49-5.51 (dd, J=10 Hz, 4.0 Hz, 1H), 5.37-5.39 (m, 1H), 4.66-4.69 (m, 1H), 3.36-3.40 (m, 1H), 2.67-2.71 (t, J=10 Hz, 1H), 2.31-2.33 (m, 1H), 2.13-2.16 (m, 1H), 2.10 (s, 3H), 1.20-2.08 (m, 14H), 0.86 (s, 3H), 0.48 (s, 3H). ESI-MS: calculated for C₂₁H₃₀O₂, 314.2, found 337.3 [M+Na]⁺.

UV and MS data of synthesized androsta- and pregna-5,7-dienes are summarized in Table 5 and the detailed NMR data are presented in Table 6. NMR chemical shifts for 5b and 5c are in agreement with those previously published (33).

TABLE 5

| | | Parental | | Predicted | |
|---|---|---|---|---|---|
| Structure type | No | compound | UV max (nm) | MW | MS+ |
| 4-6-diene | 6c | 2c | 232, 240, 249 | 314.46 | 337.3 [M + Na]+ |
| 5-7-diene | 4a | 1 | 262, 272, 281, 292 | 288.42 | 311.3 [M + Na]+ |
| | 4b | 2b | 263, 272, 282, 293 | 316.48 | 339.25 [M + Na]+ |
| | 5a | 2a | 263, 272, 281, 293 | 330.46 | 353.25 [M + Na]+ |
| | 5b | 2b | 263, 271, 282, 292 | 286.41 | 309.3 [M + Na]+ |
| | 5c | 2c | 262, 272, 283, 294 | 314.46 | 337.3 [M + Na]+ |
| preD-like | 4a-pD | 4a | 260 | 288.42 | ND^{a} |
| | 4b-pD | 4b | 260 | 316.48 | ND^{a} |
| | 5a-pD | 5a | 260 | 330.46 | ND^{a} |
| | 5b-pD | 5b | 260 | 286.41 | ND^{a} |
| | 5c-pD | 5c | 260 | 314.46 | ND^{a} |
| D-like | 4a-D | 4a | 264 | 288.42 | 311.3 [M + Na]+ |
| | 4b-D | 4b | 265 | 316.48 | 339.25 [M + Na]+ |
| | 5a-D | 5a | 265 | 330.46 | 353.25 [M + Na]+ |
| | 5b-D | 5b | 264 | 286.41 | 309 [M + Na]+ |
| | 5c-D | 5c | 265 | 314.46 | 337.3 [M + Na]+ |
| L-like | 4a-L | 4a | 262, 271, 282 | 288.42 | 311.3 [M + Na]+ |
| | 4b-L | 4b | 262, 272, 281 | 316.48 | 339.25 [M + Na]+ |
| | 5a-L | 5a | 264, 273, 281 | 330.46 | 353.25 [M + Na]+ |
| | 5b-L | 5b | 261, 272, 280 | 286.41 | ND^{a} |
| | 5c-L | 5c | ND^{a} | ND^{a} | ND^{a} |
| T-like | 4a-T | 4a | 272, 281, 291 | 288.42 | ND^{a} |
| | 4b-T | 4b | 272, 280, 291 | 316.48 | ND^{a} |
| | 5a-T | 5a | 271, 280, 290 | 330.46 | 353.25 [M + Na]+ |
| | 5b-T | 5b | 271, 280, 289 | 286.41 | 309 [M + Na]+ |
| | 5c-T | 5c | 274, 281, 290 | 314.46 | 337.3 [M + Na]+ |
| isoT-like | 5c-iT | 2c | 233, 238, 248 | 314.46 | 337.3 [M + Na]+ |
| isoT-like (oxide) | 4a-iT | 4a | 234, 251, 260 | 320.42 | 343 [M + Na]+ |
| | 5a-iT | 5aiT | 238, 249, 260 | 362.46 | 385.15 [M + Na]+ |

TABLE 6

| | CDCl3 | CDCl3 | CDCl3 | CDCl3 | CDCl3 | CD3OD |
|---|---|---|---|---|---|---|
| Solvent | 3c | 5c | 5a | 4b | 5b | 4a |
| 1 CH₂ | α 1.337 | α 1.31 | α 1.31* | α 1.31* | α 1.38 | α 1.28 |
| | β 1.89 | β 1.90 | β 1.90* | β 1.90 | β 1.91 | β 1.83 |
| 2 CH₂ | α 1.942 | α 1.92 | α 1.92* | α 1.92 | α 1.94 | α 1.8-2.2 |
| | β 1.583 | β 1.54 | β 1.54* | β 1.54* | β 1.52 | β 1.46 |
| 3 CH | 4.71 | 3.65 | 3.64 | 3.64 | 3.66 | 3.51 |
| 4 CH₂ | α 2.51 | α 2.48 | 2.48 | α 2.41 | α 2.51 | α 2.41 |
| | β 2.364 | β 2.31 | 2.31 | β 2.29 | β 2.31 | β 2.24 |
| 6 CH | 5.58 | 5.58 | 5.58 | 5.58 | 5.63 | 5.55 |
| 7 CH | 5.42 | 5.43 | 5.45 | 5.42 | 5.56 | 5.37 |
| 9 CH | 2.03 | 2.02 | 2.02* | 2.02* | 2.07 | 1.91 |
| 11 CH₂ | α 1.712 | α 1.7 | α 1.7* | α 1.7-1.8 | α 1.75 | α 1.68 |
| | β 1.788 | β 1.7 | β 1.7* | β 1.7-1.8 | β 1.75 | β 1.68 |
| 12 CH₂ | α 1.518 | α 1.49 | α 1.49* | α 1.49* | α 1.37 | α 1.18 |
| | β 2.114 | β 2.12 | β 2.12* | β 2.18 | β 1.94 | β 1.8-2.0 |
| 14 CH | 2.047 | 2.05 | 2.05 | 2.05 | 2.2 | 1.8-2.0 |
| 15 CH₂ | α 1.827 | α 1.82 | α 1.82* | α 1.82* | α 2.1 | α 1.8-2.0 |
| | β 1.543 | β 1.51 | β 1.51* | β 1.51* | β 1.79 | β 1.72 |
| 16 CH₂ | α 1.765 | α 1.76 | α 2.61 | α 1.76* | α 2.20 | α 1.55 |
| | β 2.21 | β 2.22 | 2.70 | β 2.22* | β 2.54 | β 1.8-2.0 |
| 17 CH | 2.632 | 2.63 | C | 2.17 | C | 3.69 |
| 18 CH₃ | 0.582 | 0.58 | 0.69 | 0.77 | 0.83 | 0.68 |
| 19 CH₃ | 0.947 | 0.95 | 0.78 | 0.7 | 0.98 | 0.96 |
| 20 C | | | | 3.75 CH | | |
| 21 CH₃ | 2.148 | 2.16 | 2.29 | 1.17 | | |
| 3β-Ac | 2.044 | | | | | |
| CH₃ | | | | | | |

*Chemical shifts based on similar structures. C-quaternary carbon

### General Syntheses of (5Z,7E)-9,10-secopregna-5,7,10(19)-triene-3β,20-diol (20-OH pD3) and Its Analogs

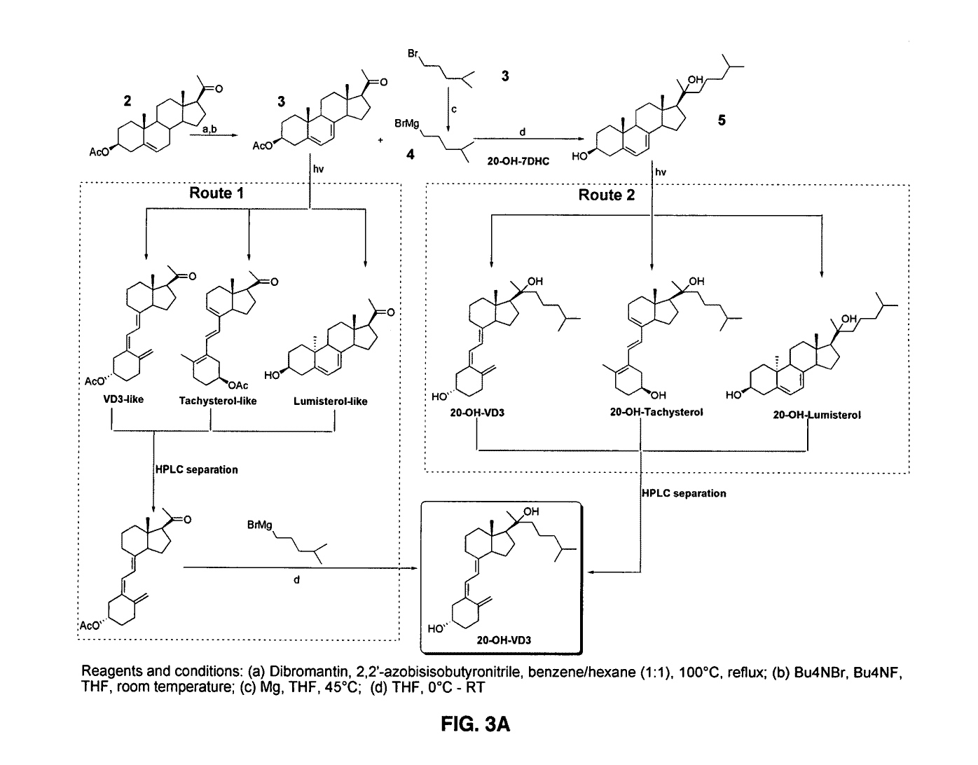

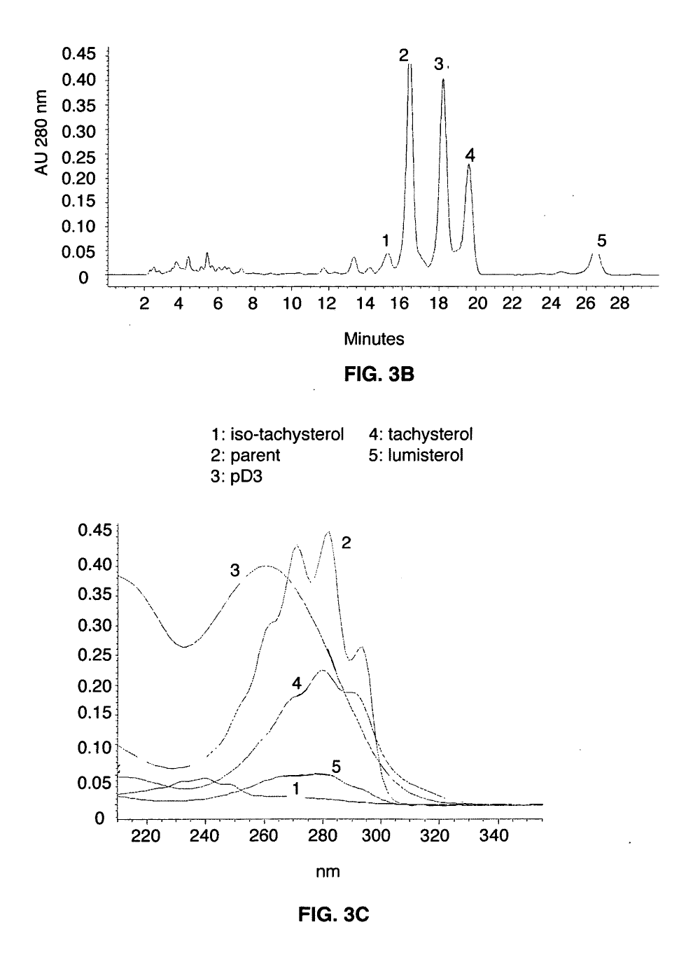

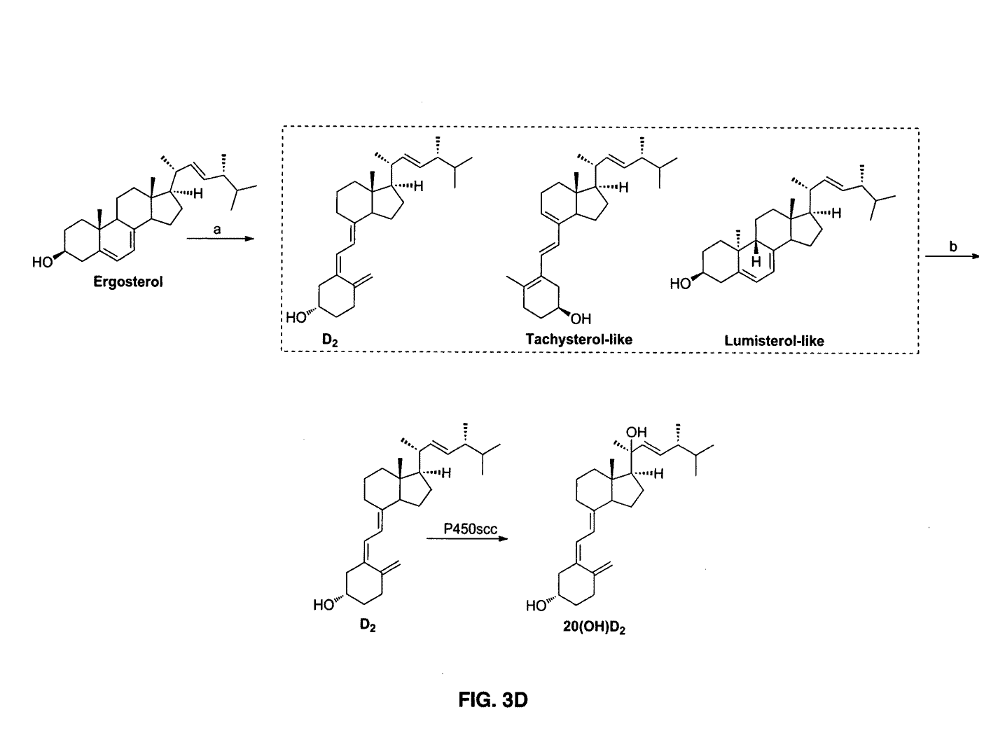

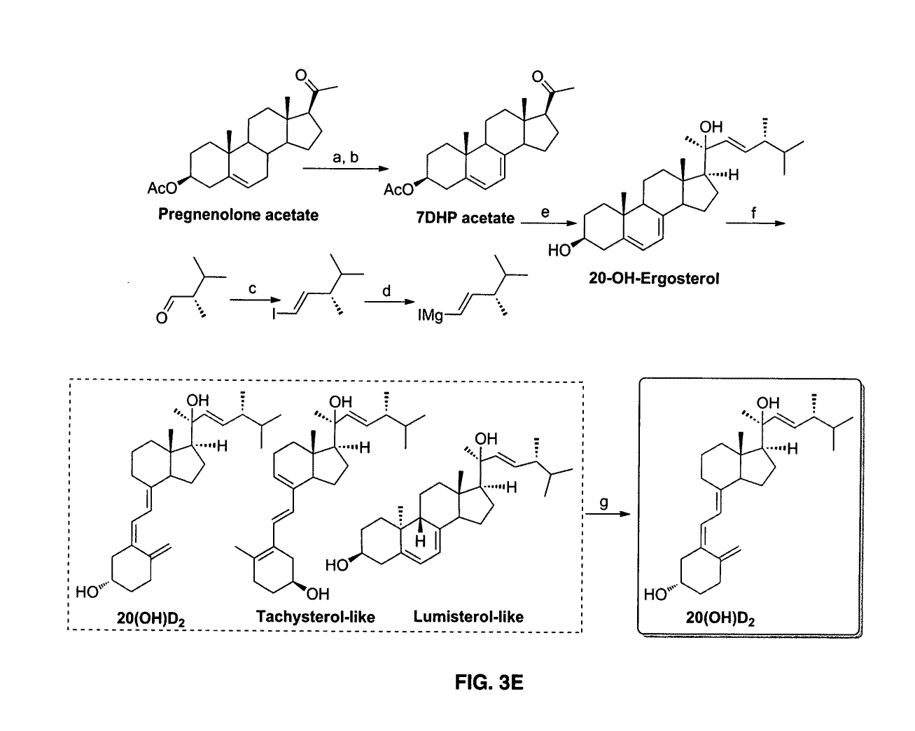

FIG. 3A depicts two routes for the chemical synthesis of (20-OH pD3). In a first synthetic route compound 3 was synthesized according to a known procedure (32) with a yield of 40-50%. Compound 4 was synthesized according to a known procedure (2). Compound 1 (1-Bromo-4-Methyl-Pentane, 3.3 g, 20.0 mmol) in dry THF (50 mL) was added drop wise to magnesium powder (735 mg, 30.0 mmol, 1.5 eq) in an argon-purged flask. The mixture was then stirred for 2 h at 45° C. The resulting solution 4 was cooled to room temperature and used for next step without further purification.

Compound 3 (712 mg, 2.0 mmol) was added to a solution of compound 4 (in excess, 20 to 30 eq) in dry THF at 0° C. under argon. The solution was allowed to warm up to room temperature and stirred overnight. The reaction mixture was quenched with saturated aqueous NH₄Cl solution and extracted with EtOAc. The organic layer was washed with brine and water, dried by MgSO₄ and concentrated. The crude material was subject to column chromatography (Hexane:EtOAc 10:1) to give a white solid compound 5 (20-OH-7DHC) at a 75% yield.

A methanol solution of compound 5 (5.0 mg, 1 mg/mL) was subjected to UV irradiation for 5 min in a quartz cuvette, using a Biorad UV Transilluminator 2000 (Biorad, Hercules, Calif.). The spectral characteristics of the UVB (280-320 nm) source were published previously³ and its strength (4.8±0.2 mW cm⁻¹) was measured routinely using a digital UVB Meter Model 6.0 (Solartech Inc., Harrison Twp, Mich.). The reaction mixture was incubated, as indicated (RT or 37° C.), for 14 hours and products were purified by RP-HPLC chromatography. The major products, i.e., the parent compound, secosteroids, lumisterols, and tachysterols were identified on the basis of their retention time (FIG. 3B) and UV absorption spectra (FIG. 3C) followed by MS and NMR measurement.

Alternatively, in a second synthetic route after synthesis of compound 3, it was subjected to UVB irradiation as described above. The photochemical reaction products are separated by RP-HPLC. The vitamin D3-like compound or its tachysterol-like and lumisterol-like analogs are subsequently reacted with a Grignard reagent that contains a proper side-chain to form pregna-5,7-diene-3b,20-diol (3D in Table 2) or its analogs.

### General Enzymatic and Chemical Syntheses of (5Z,7E,22E)-9,10-secoergosta-5,7,9(10),22-tetraene-3β,20-diol (20(OH)D₂) and Its Analogs

In FIG. 3D the natural enzymatic route starts with the UVB photolysis of ergosterol followed by binding of vitamin D₂ to P450scc and its subsequent hydroxylation to 20(OH)D₂. In an alternate chemical synthetic route pregnenolone acetate 1 is converted to 7DHP acetate 2 in a bromination/debromination and dehydrogenation reaction as for 20-OHpD₃ above. 2,3-dimethyl-n-butyraldehye is reacted with triiodomethane in the presence of chromium chloride and the product 1-iodo-3,4-dimethylpentane is added dropwise to powdered magnesium. 7DHP acetate 2 is added to the resultant product in THF at −78° C. to yield 20-OH-ergesterol which is then subjected to UV radiation as above. The photochemical reaction products, 20(OH)D2 and its tachysterol and lumisterol analogs, are separated by RP-HPLC.

### General Synthesis of (5Z,7E)-3b-hydroxy-9,10-secoandrosta-5,7,9(10)-triene-17α-carboxylic Acid and Its Analogs

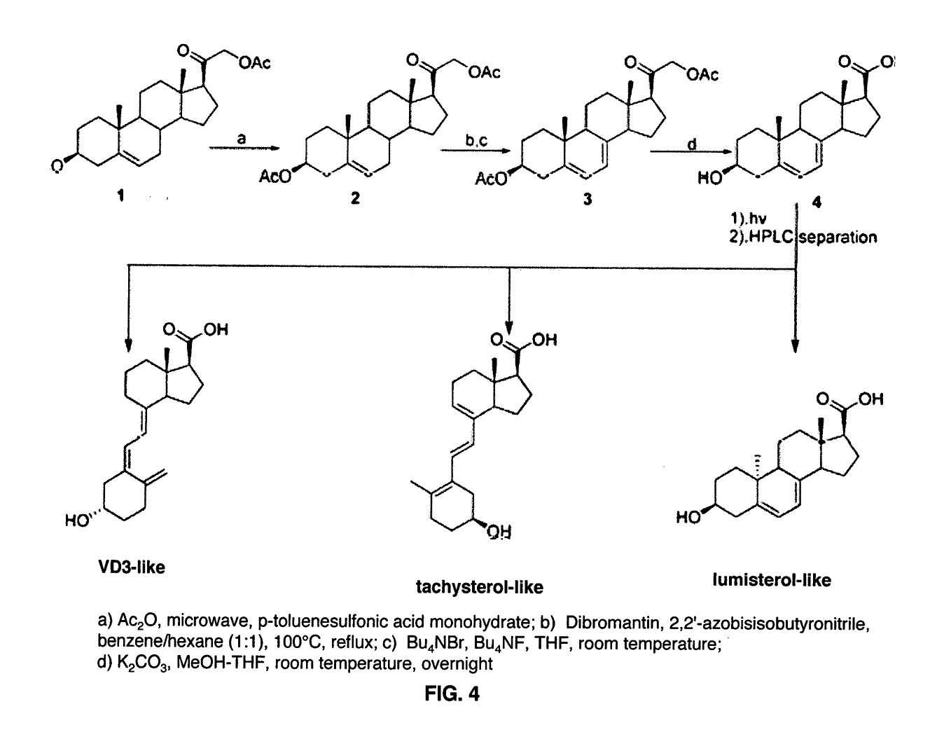

FIG. 4 depicts the chemical synthesis of 20-OH pD3. The acetylation of compound 1 was carried out following a known procedure (1). Yield: 95%. ¹H NMR (500 MHz, CDCl₃): δ 5.40 (m, 1H), 4.75 (d, J=18.0 Hz, 1H), 4.63 (m, 1H), 4.56 (d, J=18.0 Hz, 1H),2.54 (t, J=9.8 Hz, 1H), 2.33-2.36 (m, 2H), 2.25 (m, 1H), 2.18 (s, 3H), 2.06 (s, 3H), 2.01-2.08 (m, 2H), 1.87-1.90 (m, 2H), 1.40-1.76 (m, 10H), 1.30 (m, 1H), 1.18 (m, 1H), 1.04 (s, 3H), 0.70 (s, 3H). ESI-MS: calculated for C₂₄H₃₆O₅, 416.3, found 439.3 [M+Na]⁺

Compounds 3 was synthesized according to a known procedure². Yield: 40-50%. ¹H NMR (500 MHz, CDCl₃): δ 5.60 (dd, J=9.6 Hz, 2.8 Hz, 1H), 5.46 (m, 1H), 4.78 (d, J=16.0 Hz, 1H), 4.73 (m, 1H), 4.58 (d, J=16.0 Hz, 1H), 2.64 (t, J=9.6 Hz, 1H), 2.54 (m, 1H), 2.39 (t, J=14.8 Hz, 1H), 2.28 (m, 1H), 2.20 (s, 3H), 2.14 (m, 1H), 2.08 (s, 3H), 2.04-2.10 (m, 2H), 1.50-1.96 (m, 8H), 1.50 (dt, J=14.8 Hz, 8.0 Hz, 1H),1.40 (dt, J=14.0 Hz, 5.0 Hz, 1H), 0.96 (s, 3H), 0.65 (s, 3H). ESI-MS: calculated for C₂₅H₃₄O₅, 414.2, found 437.3 [M+Na]⁺.

Compound 4 was synthesized as shown in the scheme. Yield: 52%. ¹H NMR (500 MHz, CD₃OD): δ 5.57 (dd, J=7.5 Hz, 2.5 Hz, 1H), 5.44 (m, 1H), 3.54 (m, 1H), 3.19(m, 1H), 2.48 (t, J=12.6 Hz, 1H), 2.44 (m, 1H), 2.27 (t, J=15.0 Hz, 1H), 2.12-2.20 (m, 2H), 2.02-2.08 (m, 2H), 1.30-1.96 (m, 9H), 0.97 (s, 3H), 0.69 (s, 3H). ESI-MS: calculated for C₂₀H₂₈O₃, 316.2, found 315.0 [M−H]⁺.

A methanol solution of compound 5 (5.0 mg, 1 mg/mL) was subjected to UV irradiation for 5 min in a quartz cuvette, using a Biorad UV Transilluminator 2000 (Biorad, Hercules, Calif.). The spectral characteristics of the UVB (280-320 nm) source were published previously³ and it's strength (4.8±0.2 mW cm⁻¹) was measured routinely using a digital UVB Meter Model 6.0 (Solartech Inc., Harrison Twp, Mich.). The reaction mixture was incubated, as indicated (RT or 37° C.), for 14 hours and selected products were purified by RP-HPLC chromatography. The major products, pre-D-, D-, T- and L-like, were identified on the basis of their retention time and UV absorption spectra followed by MS and NMR measurement.

### EXAMPLE 3

### Physicochemical Properties of Synthesized androsta- and pregna-5.7-dienes

### UVB Irradiation of androsta- and pregna-5.7-dienes and Identification of Products

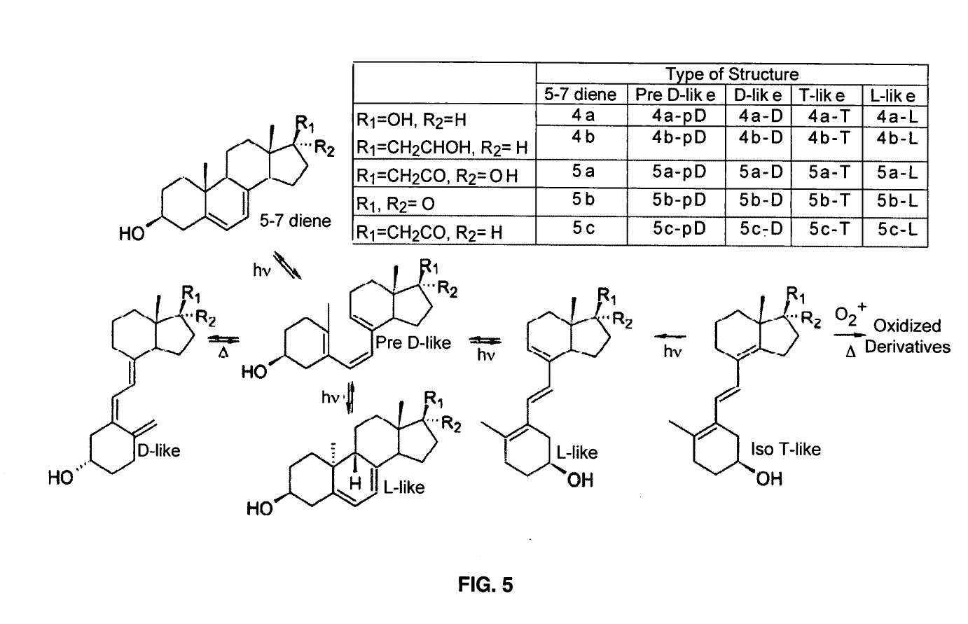

The UV conversion of androsta- and pregna-5,7-dienes were performed using a UVB light source (4.8±0.2 mW cm⁻²) with maximum emission spectrum in a range of 280-320 nm (35). The photolysis reaction and subsequent time-dependent conversion of products were monitored by a HPLC equipped with a diode array that enabled very rapid monitoring of products by characteristic UV spectra. Theoretically, four main products (FIG. 5) should be detected, based on their UV absorption, as was show n for photolysis of cholesta-5,7-dien-33-ol (7DHC). Products of irradiation were characterized based on their retention time related to the substrate and UV spectra. This enabled compounds with longer retention times to be assigned as L-like, D-like, T-like and pre-D-like (Table 5).

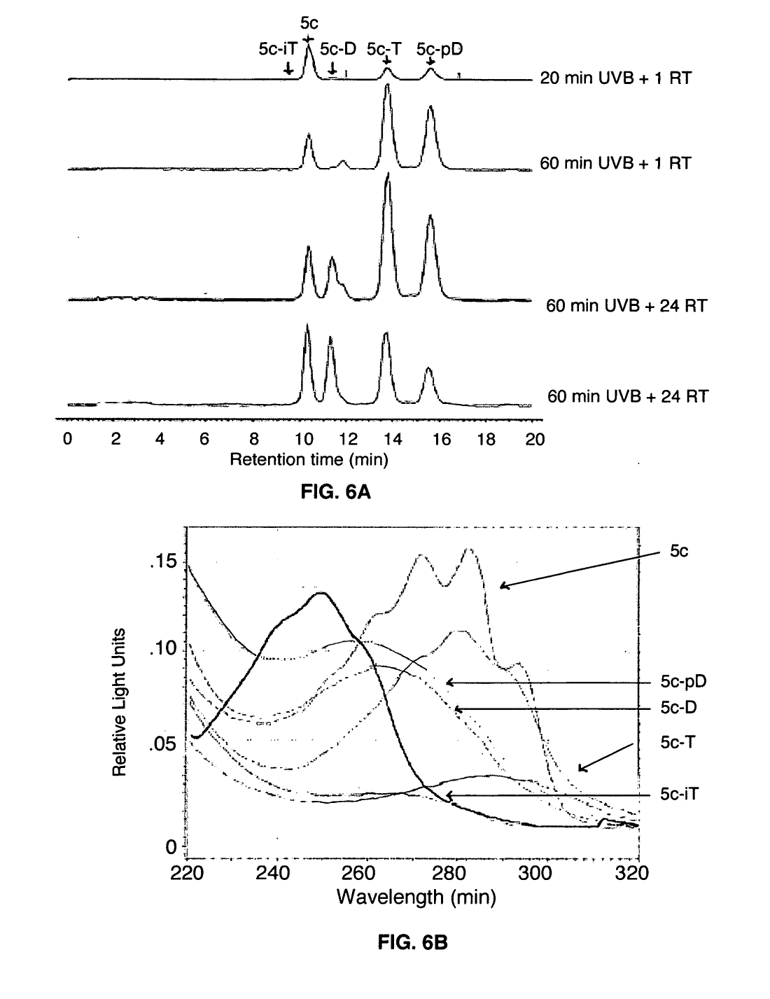

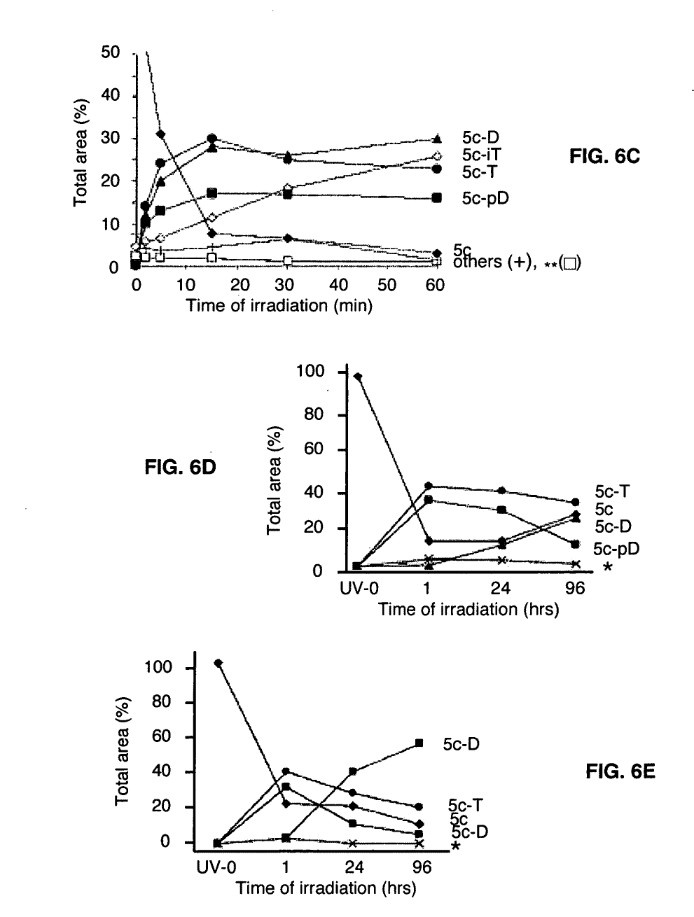

Short irradiation (20 min.; FIG. 6A) of 5c resulted in the formation of 5c-pD (λₘₐₓ at 260 nm) and subsequent slow conversion to (5Z,7E)-3β-hydroxy-9β,10α-secopregna-5,7,10(19)-trien-20-one (5c-D) with maximum UV absorption at 265 nm. Additional products were 5c-T-(λₘₐₓ at 274, 281, 290 nm) and two unknow n products with λₘₐₓ at 265 and 290 nm (FIG. 5B). The presence of 5c-L-3β-hydroxy-9β,10α-pregna-5,7-dien-20-one was not detected after irradiation of 5c (FIG. 6B). Experiments with 5c showed maximum production of 5c-pD after 15 minutes. The UVB irradiation for 30 and 60 minute resulted in an increase in a product with a λₘₐₓ at 290 (about 20%) and a slight decrease in the formation of other products (FIG. 6C).

In order to monitor changes in photolysis products of with regards to UV dose, the relative ratio between absorption at 280 nm and 240 nm was calculated for all peaks. This ratio is very useful because it enabled us to discriminate 5-7 dienes, T-like and L-like products, with λₘₐₓ close to 280 nm, from the other products with λₘₐₓ below 250 nm (isoT-like, suprasterols (34) and compounds without conjugated double bond systems). D-like compounds with λₘₐₓ at 260 and 265 nm have similar absorption at 240 and 280 nm. The ratio is close to 3 for both non-irradiated, and sham-irradiated controls and decreases to 0.11 after 60 minutes of irradiation. Thus a high UVB dose resulted in a shift of the equilibrium between main products (D-like, L-like and T-like), presumably by stimulating isomerization of T-like to isoT-like and further oxidation of isoT-like compounds. It cannot be ruled out that some of the products represent suprasterols with the λₘₐₓ below 250 nm, but in our hands the products with such spectra characteristic (λₘₐₓ 238, 249, 260 nm+/−5 nm) have the molecular w eight of parental compound (+O₂+Na+), as it was shown for 4a-iT and 5a-iT (Table 5). Unfortunately, isoT-like compounds could not be further characterized because of their low stability under test conditions.

Pre-pregnacalciferol (5c-pD) was efficiently converted to 5c-D in a time-dependent manner. Usually 4-7 days at room temperature was sufficient for this conversion (FIG. 6D). Incubation at 37° C. effectively accelerated this process (FIG. 6E). Interestingly, higher temperature not only stimulated the conversion, but also moved equilibrium towards 5c-D formation, with decreases in other products.

Irradiation of other 5-7 dienes (compounds: 4a, 4b, 5a and 5b) resulted in similar pattern of products and UVB dose- and time-dependent conversions. Further identification was performed after purification by HPLC and the corresponding fractions of the selected peaks were analyzed by mass spectrometry. As predicted all D-like, L-like and T-like products had identical molecular weight corresponding to androsta- or pregna-5,7-diene precursor (Table 5).

### Identification of L-Like, D-Like, and T-Like Compounds by NMR

The D-, L- or T-like irradiation products of androsta- and pregna-5,7-dienes of defined UV and mass spectra were subjected to NMR. The assignment of structures is based on ¹H -NMR data and selected 2D experiments (COSY, TOCSY and HSQC). Table 7 shows the 13C and 1H NMR chemical shifts of vitamin D-like compounds and T-like (5aD) compound. Table 8 shows ¹H NMR chemical shifts of L-like compounds. Identification was assigned based on expected chemical shifts and presence or absence of vinylic protons 6-CH and 7-CH; and methyl groups at C18, C19 and C21.

TABLE 7

| | ¹³C5c-D | ¹³C4a-D | ¹H5c-D | ¹H5a-D | ¹H5a-T | ¹H4b-D | ¹H5b-D | ¹H4a-D |
|---|---|---|---|---|---|---|---|---|
| 1 CH₂ | 23.12 | 33.333 | α 2.12 | α 2.11 | α 2.11* | α 2.11 | α 2.17 | α 2.12 |
| | | | β 2.41 | β 2.41 | β 2.41* | β 2.41 | β 2.42 | β 2.41 |
| 2 CH₂ | 35.5 | 36.372 | α 1.93 | α 1.97 | α 1.97* | α 1.97 | α1.97* | α1.97 |
| | | | β 1.68 | β 1.68 | β 1.68* | β 1.54 | β 1.54* | β 1.54 |
| 3 CH—OH | 69.99 | 70.345 | 3.96 | 3.76 | 3.86 | 3.76 | 3.8 | 3.77 |
| 4 CH₂ | 46.5 | 46.806 | α 2.58 | α 2.53 | α 2.53* | α 2.55 | α 2.59 | α 2.54 |
| | | | β 2.30 | β 2.19 | β 2.19* | β 2.20 | β 2.24 | β 2.19 |
| 6 CH | 122.2 | 122.282 | 6.22 | 6.23 | 6.58 | 6.22 | 6.27 | 6.23 |
| 7 CH | 118.98 | 118.73 | 6.06 | 6.08 | 6.39 | 6.02 | 6.19 | 6.04 |
| 9 CH2 | 29.05 | 29.573 | 2.85 | 2.87 | CH | 2.87 | 2.94 | 2.89 |
| | | | | | 5.35 | | | |
| | | | 1.72 | 1.5 | | 1.56 | 1.67* | 1.67 |
| 11 CH₂ | 23.3 | 23.938 | α 1.77 | α 1.75 | α 1.75* | α 1.68 | α 1.71* | β 1.71 |
| | | | β 1.77 | β 1.75 | β 1.75* | β 1.67 | β 1.53* | β 1.53 |
| 12 CH₂ | 39.75 | 38.457 | α 1.56 | α 1.50 | α 1.50* | α 1.34 | α 1.24* | α 1.24 |
| | | | β 2.05 | β 2.06 | β 2.06 | β 1.52 | β 1.86* | β1.86 |
| 14 CH | 56.7 | 51.882 | 2.12 | 2.14 | 2.14* | 2.02 | β2.47 | β 1.99 |
| 15 CH₂ | 22.46 | 22.045 | α 1.61 | α 1.6 | α1.6* | α 1.50 | α 1.45* | α 1.45 |
| | | | β 1.61 | β 1.6 | β 1.6* | β 1.50 | β 1.64* | β 1.64 |
| 16 CH₂ | 22.61 | 29.979 | α 1.7 | α 1.68 | α 1.68* | α 1.63 | α 1.46* | α 1.46 |
| | | | β 2.17 | β 2.76 | β 2.76* | β 2.17 | β 2.47 | β 2.06 |
| 17 CH | 64.25 | 82.799 | 2.7 | N/A | N/A | 1.51 | N/A | 3.74 |
| 18 CH3 | 13.04 | 11.348 | 0.51 | 0.46 | 0.883 | 0.61 | 0.75 | 0.58 |
| 19 CH₂ | 113.7 | 112.492 | α 4.81 | α 4.75 | CH₃ | α 4.74 | 4.82 | 4.75 |
| | | | | | 1.79 | | | |
| | | | β 5.06 | β 5.05 | N/A | β 5.04 | 5.1 | 5.04 |
| 20 C | ND | ND | N/A | N/A | ND | 3.62 | N/A | N/A |
| 21 —CH₃ | 31.47 | | 2.143 | 2.223 | 2.23 | 1.12 | N/A | N/A |

*Chemical shifts based on similar structures.
ND—Not determined;
N/A—Not applicable (ternary carbons)

TABLE 8

| | 5a-L | 4b-L | 4a-L |
|---|---|---|---|
| 1 CH₂ | α 1.31* | α 1.31* | α 1.296 |
| | β 1.90* | β 1.90* | β 1.771 |
| 2 CH₂ | α 1.92* | α 1.92* | α 1.711 |
| | β 1.54* | β 1.54* | β 1.616 |
| 3 CH | 4.03 | 4.03 | 4.033 |
| 4 CH₂ | α 2.44 | α 2.44 | α 2.43 |
| | β 2.28 | β 2.35 | β 2.264 |
| 6 CH | 5.584 | 5.57 | 5.423 |
| 7 CH | 5.488 | 5.42 | 5.58 |
| 9 CH | 2.02* | 2.02* | 2.34 |
| 11 CH₂ | α 1.7-1.8* | α 1.7-1.8* | α 1.49 |
| | β 1.7-1.8* | β 1.7-1.8* | β 1.49 |
| 12 CH₂ | α 1.49* | α 1.49* | α 1.51 |
| | β 2.18* | β 2.18* | β 1.91 |
| 14 CH | 2.05* | 2.05* | 2.48 |
| 15 CH₂ | α 1.82* | α 1.82* | α 1.667 |
| | β 1.51* | β 1.51* | β 1.575 |
| 16 CH₂ | α 2.68 | α 1.76* | α 1.501 |
| | β 2.76 | β 2.22* | β 2.083 |
| 17 CH | N/A | 2.17* | 3.833 |
| 18 CH₃ | 0.517 | 0.77 | 0.691 |
| 19 CH₃ | 0.777 | 0.7 | 0.754 |
| 20 C | N/A | 3.65 CH | |
| 21 CH₃ | 2.183 | 1.13 | |

Chemical shifts based on similar structures.
ND—Not determined; N/A—Not applicable (ternary carbons)

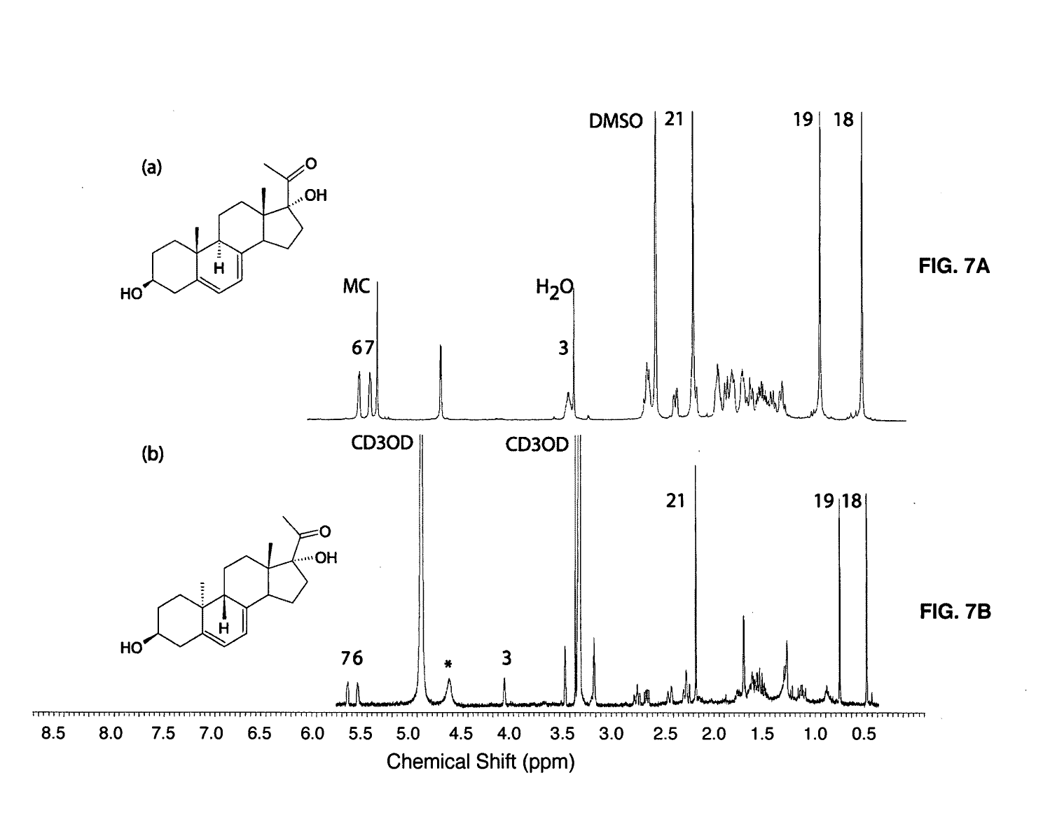

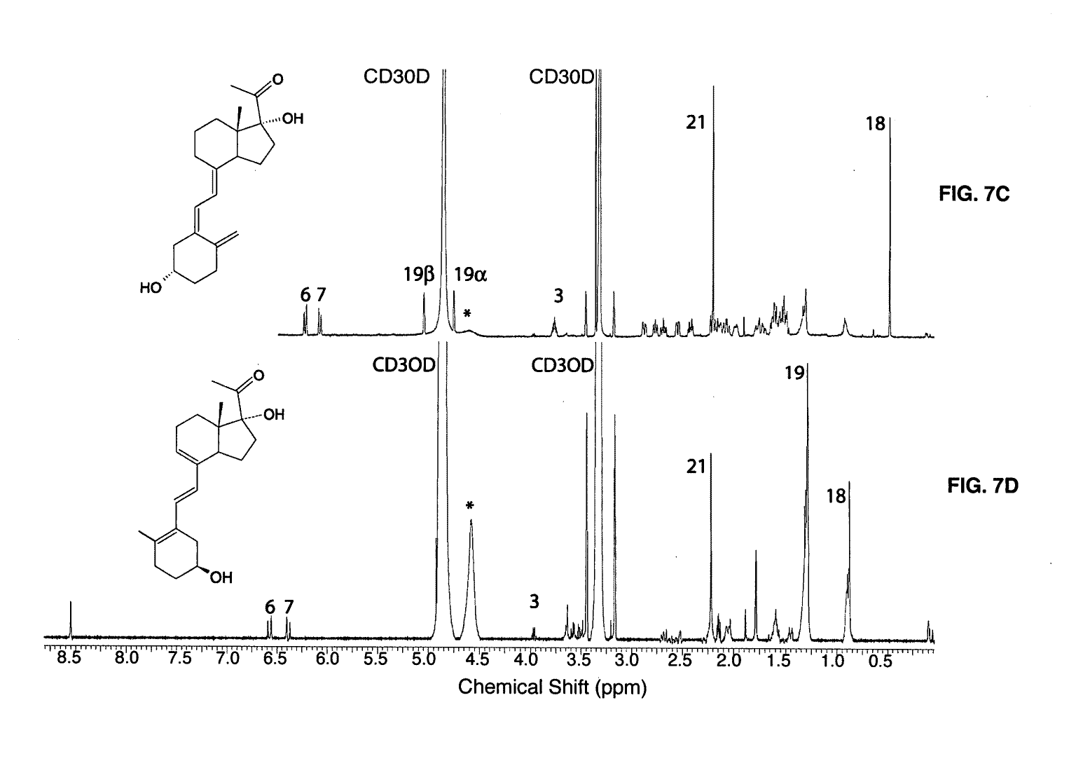

Structures of L-like derivatives (4aL, 4bL and 5aL) were confirmed based on different chemical shifts for the methyl group 19-CH₃, which was shifted downfield about 0.20 ppm (+/−0.05 ppm) when compare with their precursors. Although T-like and isoT-like compounds derived from androsta- and pregna-5,7-dienes were detected and characterized, these compounds were very reactive and unstable in deuterated chloroform. Thus, only one structure (5a-T) was confirmed by NMR (FIGS. 7A-7D), despite the fact that compounds 4a-T, 4b-T, 5b-T and 5c-T were clearly identified by characteristic UV absorbance with λₘₐₓ at 272, 280 and 290 nm (+/−2 nm). Since the presence of trace amount of HCI in 99.99% deuterated chloroform induced very fast structural degradation, the structure of T-like compounds was analyzed using deuterated methanol as solvent.

### UVB Irradiation of pregna-5.7-diene-3β,17α20-triols and Identification of Products

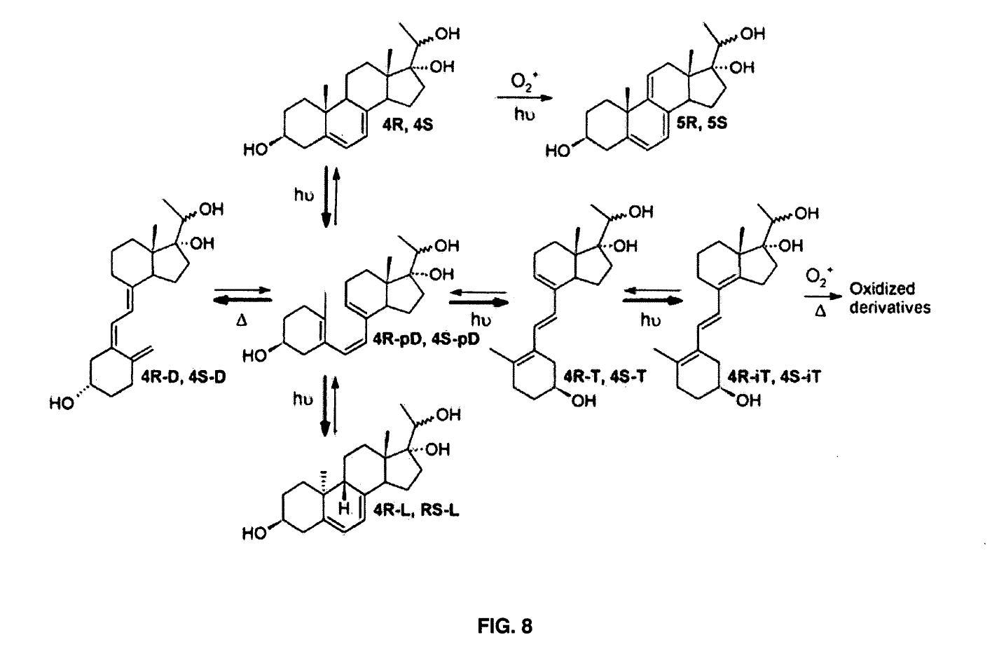

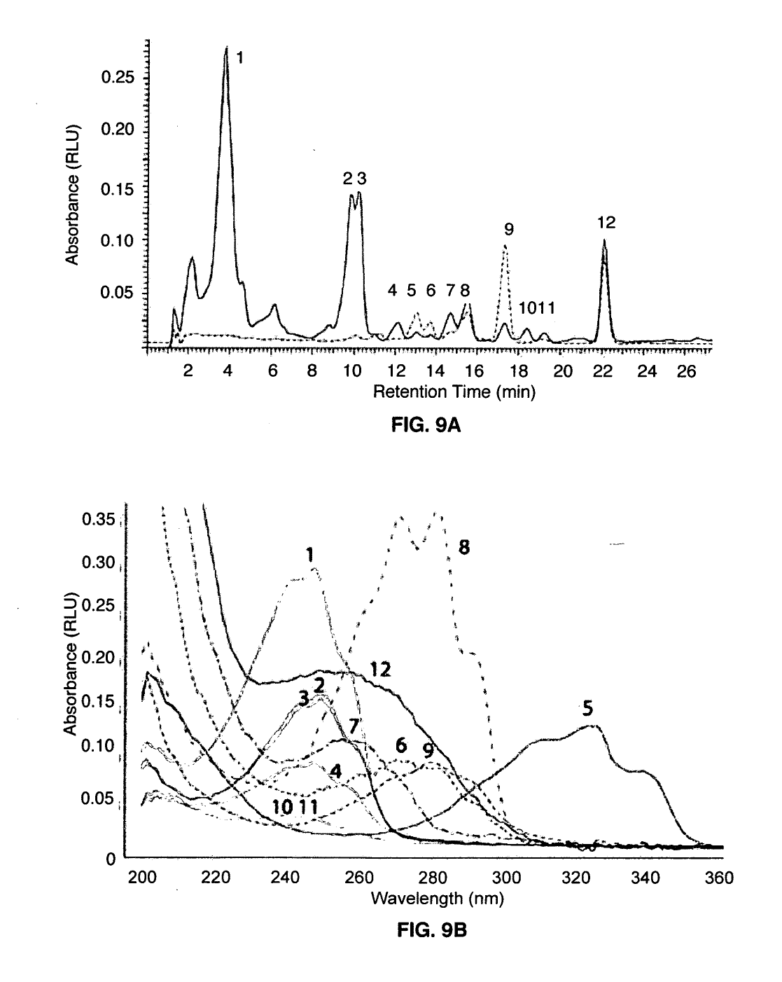

The UV conversion of 4R and 4S were performed using a UVB light source (4.8±0.2 mW cm⁻²) with maximum emission spectrum in a range of 290-320 nm (35). The photo-conversion and subsequent time dependent structural rearrangements were monitored by an HPLC equipped with a diode array that enabled fast detection of products by characteristic UV spectra (38). Similarly to cholesta-5,7-dien-3β-ol (7DHC) (16), four main products (FIG. 8) were formed in time-dependent fashion, namely pro-D-like, T-like, L-like and D-like. All products were separated by HPLC and identified based on their unique UV absorption spectra (FIGS. 9A-9B).

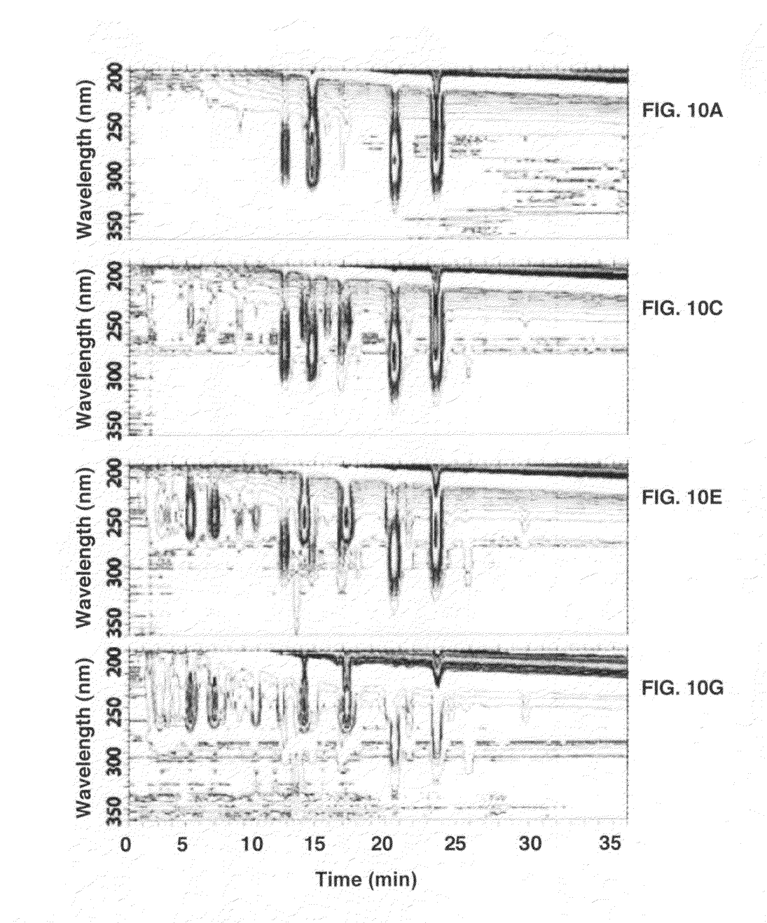

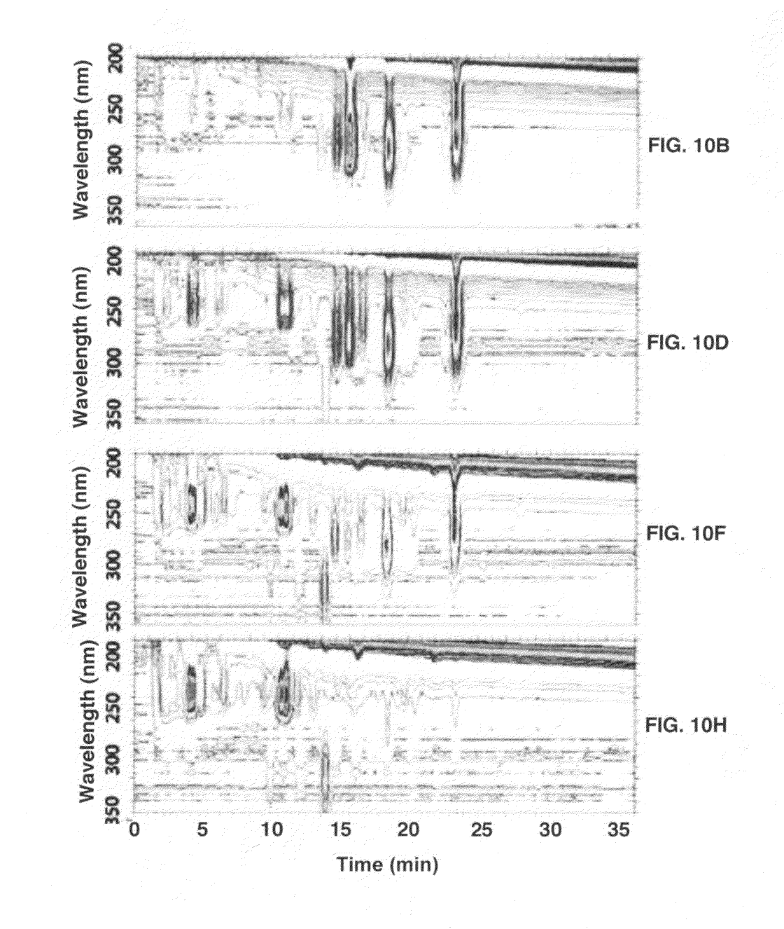

The irradiation of 4R and 4S with increasing UV doses resulted in the formation of diverse products. Short irradiation (5 min.; FIGS. 10A-10B) of both 4R and 4S resulted in the formation of pre-D-like (λₘₐₓ at 260 nm; 4R-pD and 4S-pD), T-like (λₘₐₓ at 274, 281, 290 nm 4R-T and 4S-T) and L-like (262, 271, 281; 4R-L and 4S-L) products. The products 4R-pD and 4S-pD underwent subsequent slow isomerization into vitamin D-like compounds (4R-D and 4S-D) with maximum UV absorption at 265 nm. Longer UVB irradiation (15 minutes; FIGS. 10C-10D) resulted in conversion of T-like products to isoT-like compounds with characteristic UV absorption for a conjugated double bond (λₘₐₓ at 248 nm). Irradiation for 30 and 60 minutes (FIGS. 10E-10H) resulted in further conversion into numerous iso-T-like products with a corresponding decrease in pre-D-like and T-like products, indicating further metabolism (FIG. 2) with the potential formation of suprasterols with a λₘₐₓ below 250 nm.

Further identification was performed after purification by RP-HPLC and the corresponding fractions were analyzed by MS. As expected, all D-like, L-like and T-like products had identical mass (m/z=355.25 [M+Na]⁺) with the parent compounds (Table 9). In addition to a molecular ion at m/z=355.25 [M+Na]+ the majority of iso-T-like products of irradiation had an additional ion at m/z=387.15. This indicates either the addition of O₂ with formation of peroxide or hydroxyperoxide derivatives similar to those shown for iso-T3 ((6E)-9,10-secocholesta-5(10),6,8(14)-trien-3b-ol), or oxidation of 4S and 4R without photolysis of the B ring, with production of of endoperoxide and hydroperoxide, as was shown for 7-DHC. The small scale of reaction and instability of isoT-like compounds prevented their more complete characterization.

TABLE 9

| | Parent | | | Predicted | |
|---|---|---|---|---|---|
| No | cmpd. | Structure type | UV max (nm) | MW | MS+ |
| 4R | 1 | 5,7-diene | 261, 270, 281, 290 | 332.25 | 355.25 [M + Na]+ |
| 4R1 | 4R | preD-like | 260 | 332.25 | ND |
| 4R-D | 4R | D-like | 265 | 332.25 | 355.25 [M + Na]+ |
| 4R-L | 4R | L-like | 262, 271, 281 | 332.25 | 355.25 [M + Na]+ |
| 4R-T | 4R | T-like | 270, 279, 291 | 332.25 | 355.25 [M + Na]+ |
| 4R-iT | 4R | isoT-like (oxide) | 240, 248, 257 | 364.25 | 387.15 [M + Na]+ |
| 4R7 | 4R | 5,7,9(11) triene | 312, 323, 340 | 330.22 | ND |
| 4S | 1 | 5,7-diene | 263, 271, 282, 292 | 332.25 | 355.25 [M + Na]+ |
| 4S1 | 4S | preD-like | 260 | 332.25 | ND |
| 4S-D | 4S | D-like | 265 | 332.25 | 355.25 [M + Na]+ |
| 4S-L | 4S | L-like | 262, 271, 282 | 332.25 | 355.25 [M + Na]+ |
| 4S-T | 4S | T-like | 272, 282, 297 | 332.25 | 355.25 [M + Na]+ ND |
| 4S-iT1 | 4S | isoT-like | 241, 248, 257 | 332.25 | 355.25 [M + Na]+ |
| 4S-iT2 | 4S | isoT-like (oxide) | 241, 248, 257 | 332.25 | 387.15 [M + Na]+ |
| 4S7 | 4S | 5,7,9(11) triene | 312, 323, 340 | 330.22 | ND |

Bold-purified and characterized (NMR), italics-characterized by UV spectra, ND—not determined

### Identification of L-Like, D-Like, and T-Like Compounds by NMR

The D-, L- or T-like irradiation products of 4R and 4S of defined UV and mass spectra were subjected to NMR analysis. Elucidation of the structures was based on ¹H-NMR data and selected 2D experiments (COSY, TOCSY). The detailed list of chemical shifts with an assignment of signals is shown in Table 10. The D-like (4R-D and 4S-D), and L-like (4R-L and 4S-L) compounds were assigned based on expected chemical shifts for vinylic and methyl protons with the characteristic pattern. The main difference between NMR data for L-like compounds (4R-L and 4S-L) and their respective parental compounds is a downfield shift of the methyl group at C19 (˜0.20 ppm). Although T-like and isoT-like compounds derived from 4R and 4S were detected and characterized initially, these compounds were not stable, which prevented their in-depth characterization.

TABLE 10

| | 4R | 4S | 4R-D | 4S-D | 4R-L | 4S-L | 5S |
|---|---|---|---|---|---|---|---|
| 1 CH₂ | α 1.31 | α 1.29 | α 2.12 | α 2.12 | α 1.31 | α 1.31 | 1.48 |
| | β 1.90 | β 1.90 | β 2.42 | β 2.41 | β 1.90 | β 1.90 | 1.70 |
| 2 CH₂ | α 1.92 | α 1.93 | α 1.97 | α 1.97 | α 1.92 | α 1.92 | 1.89 |
| | β 1.45 | β 1.54 | β 1.68 | β 1.68 | β 1.54 | β 1.54 | 1.68 |
| 3 CH | 3.51 | 3.51 | 3.76 | 3.76 | 4.03 | 4.03 | 3.47 |
| 4 CH₂ | α 2.41 | α 2.40 | α 2.55 | α 2.54 | α 2.41 | α 2.44 | 2.34 |
| | β 2.23 | β 2.23 | β 2.19 | β 2.19 | β 2.29 | β 2.35 | 2.42 |
| 6 CH | 5.54 | 5.54 | 6.23 | 6.24 | 5.59 | 5.54 | 5.65 |
| 7 CH | 5.42 | 5.39 | 6.09 | 6.09 | 5.47 | 5.39 | 5.40 |
| 9 CH | 2.11 | 2.02 | 2.85 | 2.85 | 2.02 | 2.02 | |
| | | | 1.54 | 1.56 | | | |
| 11 CH₂ | α 1.67 | α 1.6-1.8 | α 1.77 | α 1.77 | α 1.7-1.8 | α 1.7-1.8 | 5.61 |
| | β 1.75 | β 1.6-1.8 | β 1.77 | β 1.77 | β 1.7-1.8 | β 1.7-1.8 | |
| 12 CH₂ | α 1.49 | α 1.49 | α 1.54 | α 1.56 | α 1.49 | α 1.49 | 2.61 |
| | β 1.80 | β 2.12 | β 2.05 | β 2.04 | β 2.18 | β 2.18 | 2.14 |
| 14 CH | 2.62 | 2.57 | 2.12 | 2.12 | 2.05 | 2.05 | 2.78 |
| 15 CH₂ | α 1.83 | α 1.84 | α 1.54 | α 1.61 | α 1.82 | α 1.82 | 1.85 |
| | β 1.63 | β 1.56 | β 2.53 | β 1.61 | β 1.51 | β 1.51 | 1.50 |
| 16 CH₂ | α 1.56 | α1.7-1.8? | α 1.87 | α 1.7 | α 1.76 | α 1.76 | 1.60 |
| | β 1.74 | β 1.9? | β 2.7 | β 2.66 | β 2.22 | β 2.22 | 1.75 |
| 18 CH₃ | 0.7 | 0.77 | 0.6 | 0.67 | 0.76 | 0.76 | 0.67 |
| 19 CH₃ | 0.94 | 0.95 | α 4.76 | α 4.74 | 0.67 | 0.75 | 1.24 |
| | | | β 5.05 | β 5.05 | | | |
| 20 C | 3.78 CH | 3.94 CH | 3.76 | 3.61 | 3.72 CH | 3.65 CH | 3.9 |
| 21 CH₃ | 1.19 | 1.15 | 1.18 | 1.14 | 1.14 | 1.15 | 1.16 |

Italics - not fully determined presumably similar to parent compound.

### Detection and Characterization of Triene-Like Products of UVB Irradiation of 4R and 4S

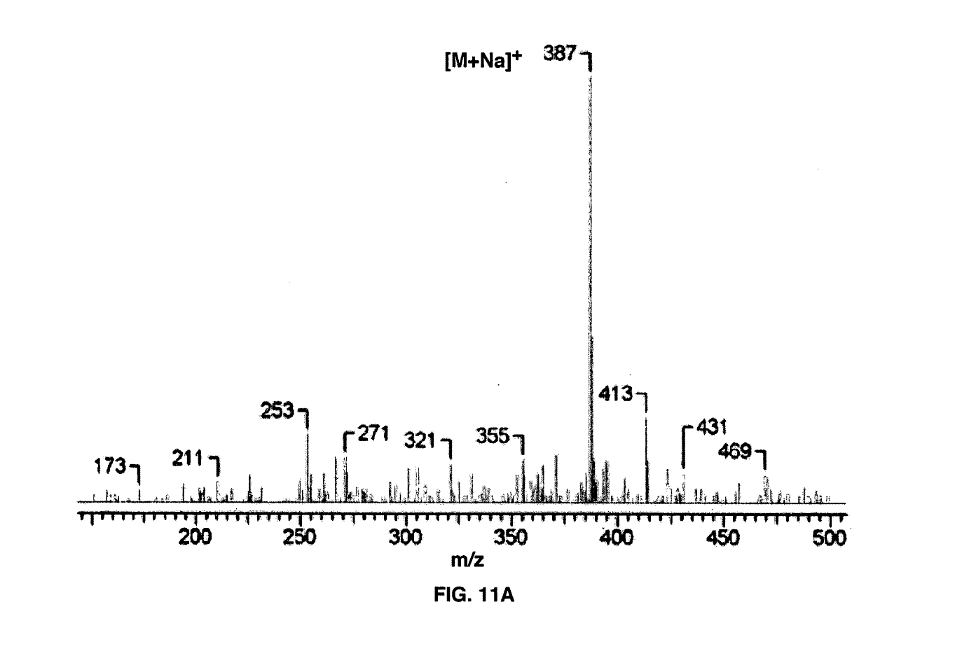

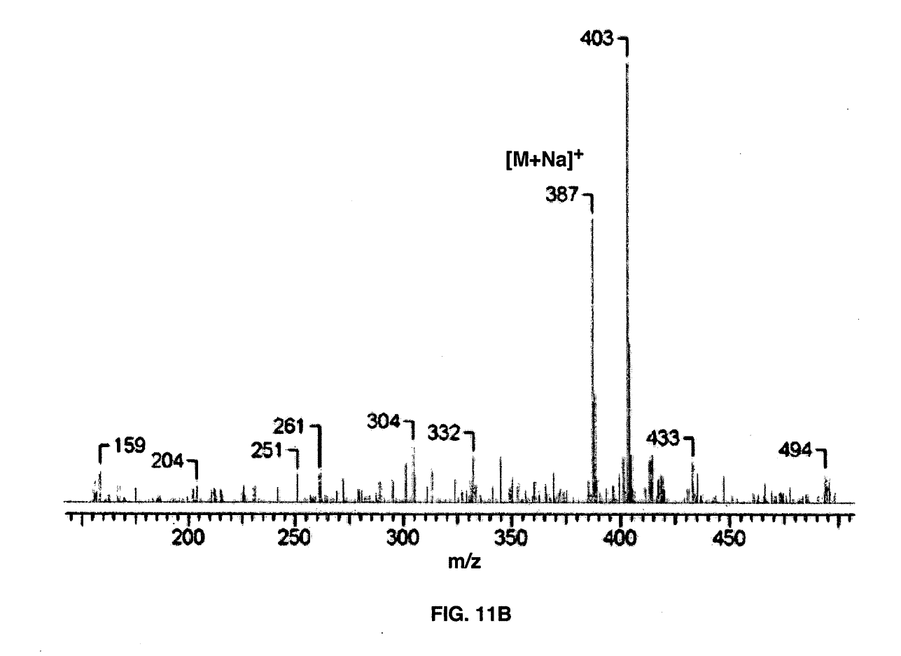

In addition to well-characterized products of 5,7-diene irradiation (D-like, L-like, T-like and isoT-like compounds), other products with UV absorption (λₘₐₓ) at 312, 232 and 240 nm were detected. This shift in UV absorption suggests the presence of a triene system, presumable similar to cholesta-5,7,9(11)-trien-3β-ol (9-DDHC), with reported λₘₐₓ at 324 nm. Although irradiation of both 4R and 4S resulted in the formation of compounds with λₘₐₓ max above 300 nm, the process was more efficient from 4S precursor (FIGS. 11A-11B). NMR analysis of this product confirmed the presence of 5,7,9(11)-triene system, and the product was identified as pregna-5,7,9(11)-triene-3β,17α,20S-triol, a compound that was previously reported (32). The chemical shifts observed for 5S (Table 10) are essentially identical (with small changes due to different solvent) to these earlier data.

### EXAMPLE 4

### Cytochrome P450scc Production of Hydroxylated Cholecalciferol Metabolites

### Metabolism in 2-hydroxypropyl-3-cyclodextrin

Adrenodoxin reductase and cytochrome P450scc were purified from bovine adrenal mitochondria (39-40). Adrenodoxin was expressed in Escherichia coli and purified as described before (41). Substrates, 1α-hydroxycholecalciferol, cholecalciferol, or 1α-hydroxycholecalciferol derivatives, were dissolved initially in 45% cyclodextrin (2-hydroxypropyl-β-cyclodextrin) which is typically 5 μM (De Caprio, 1992). Substrate in 45% cyclodextrin cytochrome P450scc (0.2-2 μM), 15 μM adrenodoxin, 0.2 μM adrenodoxin reductase, 2 mM glucose 6-phosphate, 2 U/ml glucose 6-phosphate dehydrogenase and 50 μM NADPH were added to a buffer comprising 20 mM HEPES (pH 7.4), 100 mM NaCl, 0.1 mM dithiothreitol and 0.1 mM EDTA for a final cyclodextrin concentration of 0.45%. Samples (typically 0.25-1.0 ml) were pre-incubated for 8 min at 37° C. then the reaction started by the addition of NADPH. Samples were incubated at 3TC with shaking for various times then reactions were stopped by the addition of 2 ml ice-cold dichloromethane and vortex mixing. The lower phase was retained and the upper aqueous phase was extracted twice more with 2 ml aliquots of dichloromethane. The solvent was removed under nitrogen and samples were dissolved in 64% methanol in water for HPLC analysis. Metabolites were analysed using a Perkin Elmer HPLC equipped with a C18 column (Brownlee Aquapore, 22 cm×4.6 mm, particle size 7 μm). Samples were applied to the column in 64% methanol and eluted with a 64-100% methanol gradient in water, at flow rate 0.5 ml/min. Products were detected using a UV monitor at 265 nm.

1α-hydroxycholecalciferol gave a k_{cat} of 1.3±0.1 mol/min/mol P450scc and a Kₘ of 41±6 μM when dissolved in cyclodextrin to a final concentration of 0.45%. This compares to values of 19.7±0.9 mol/min P450scc and 30±2 μM for k_{cat} and Kₘ, respectively, for cholecalciferol in this system.

### Large-Scale Preparation of Metabolites for NMR

20-Hydroxycholecalciferol was prepared enzymatically from 50 ml incubations of 2 μM P450scc with 100 μM vitamin D3 in 0.9% cyclodextrin in a scaled-up version of the method described above, and purified by preparative TLC. 20,23-Dihydroxy cholecalciferol (90 μg) and 17α,20,23-trihydroxy cholecalciferol (60 μg) were similarly prepared from 50 ml incubations of 50 μM TLC-purified 20-hydroxyvitamin D3 with 1 μM P450scc in 0.45% cyclodextrin. The two products were purified by HPLC as described above, approximately 10-20 μg at a time. UV spectra of products were recorded to check that they had the same typical cholecalciferol spectrum as the substrate and were quantitated using an extinction coefficient of 18,000 M⁻¹cm⁻¹ at 263 nm. Initial NMR of the trihydroxy cholecalciferol indicated the presence of some impurities so the sample was further purified by reverse-phase HPLC on an Atlantis C18 column (Waters Associates, Milford, Mass.) running an isocratic mobile phase of 62.5% methanol in water at 1.5 ml/min. This step removed three minor contaminants. A separate enzymatic synthesis of 20,23-dihydroxycholecalciferol (80 μg) for structure determination by NMR was performed using a 50 ml incubation of 50 μM vitamin D3 with 2 μM P450scc in 0.45% cyclodextrin, with the product being purified by TLC, then by gradient HPLC as above.

1α,20-dihydroxycholecalciferol was prepared enzymatically from a 40 ml incubations of 2 μM P450scc with 50 μM 1α-hydroxycholecalciferol (Sigma) in 0.45% cyclodextrin, in a scaled-up version of the method described above. The 1α,20-dihydroxyvitamin D3 was purified by preparative TLC using three developments of the silica gel G plate in hexane:ethyl acetate (1:1), similar to the purification of vitamin D3 metabolites (8-9). The resulting 1α,20-dihydroxycholecalciferol was further purified by preparative HPLC using a Brownlee Aquapore column (25 cm×10 mm, particle size 20 μm) and elution with a methanol gradient in water (64% to 100% methanol). This yielded 180 μg pure product of which 150 μg was used for NMR. The UV spectrum of the product was the same as the 1α-hydroxyvitamin D3 substrate.

### Metabolism of Vitamin D3 by P450scc

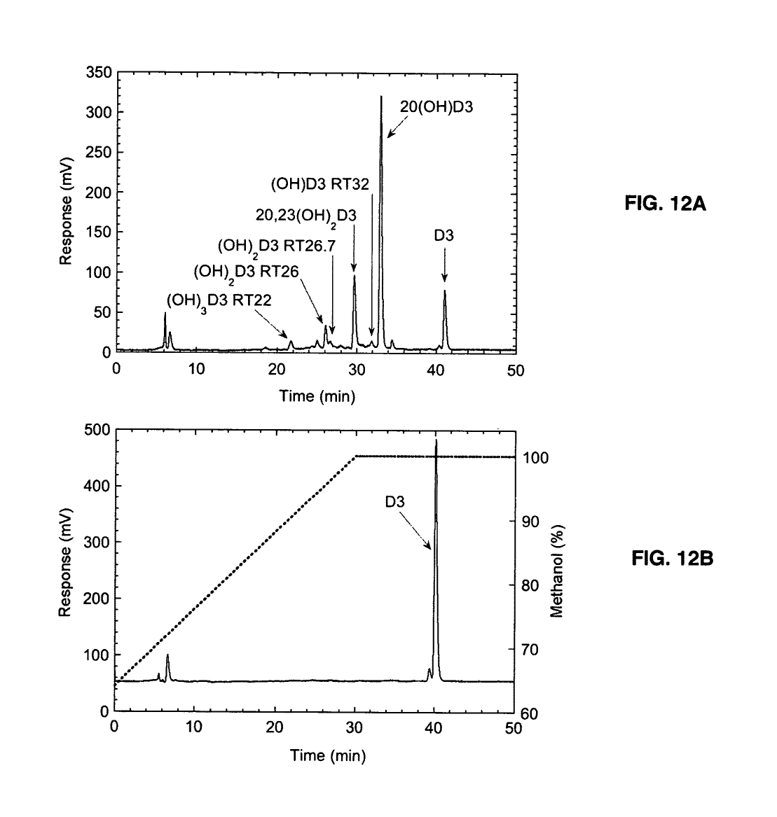

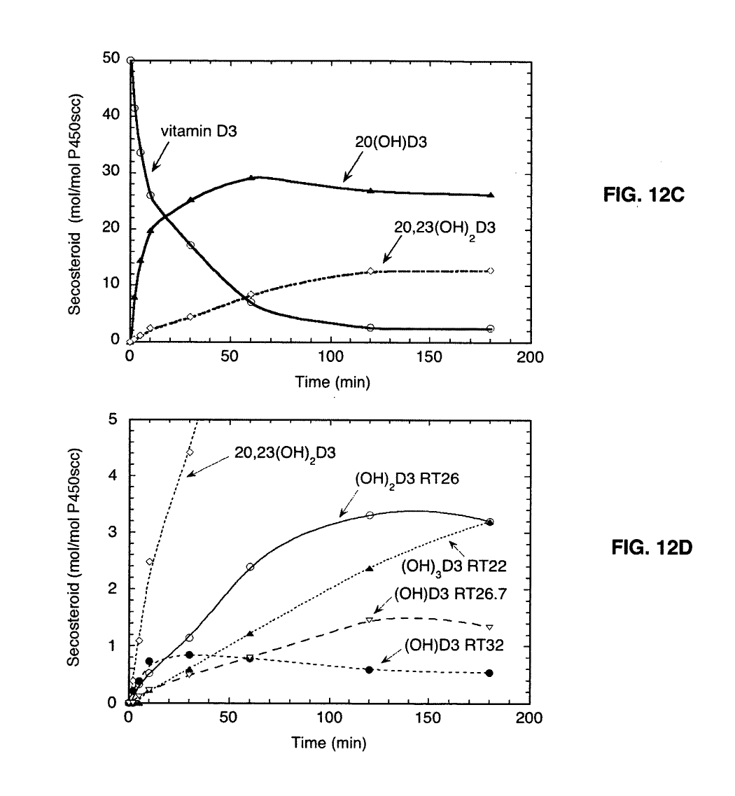

Six different products, in sufficient amounts to permit quantitation and subsequent characterization, were observed when vitamin D3 was incubated with P450scc in 0.45% cyclodextrin. A typical chromatogram of these products after a one-hour incubation is shown in FIG. 12A, together with a zero-time control (FIG. 12B). A time course for the metabolism of vitamin D3 by cytochrome P450scc in cyclodextrin is shown in FIGS. 12C-12D. The major product was 20-hydroxycholecalciferol (retention time (RT)=33 min), previously identified from authentic standard. Another major product was subsequently shown to be 20,23-dihydroxycholecalciferol (RT=30 min). In addition, four other products with retention times of 32, 26.7, 26 and 22 min (FIG. 12A) were observed in sufficient amounts throughout the time course to permit quantitation.

The electrospray mass spectrum for the product with RT =22 min showed the major ion at m/z=455.4 (432.4+Na+) and thus arises from trihydroxycholecalciferol. A major ion at m/z=887.6 corresponded to Na+ complexed to two trihydroxycholecalciferol molecules. The electrospray mass spectrum for the product with RT=26 min in FIG. 12B gave the major ion as m/z=439.4 (416.4+Na+) from which the sample was identified as dihydroxycholecalciferol. A major ion was also observed at m/z=855.7, which corresponded to Na+ complexed to two dihydroxyvitamin D3 molecules. The electrospray mass spectrum for the product with RT=32 min gave the major ion as m/z=423.4 (400.4+Na+) from which the sample was identified as monohydroxycholecalciferol. An ion at m/z=439.4 corresponded to hydroxycholecalciferol complexed to K+ (400.4+39), while an ion at m/z=823.5 corresponded to Na+ complexed to two hydroxyvitamin D3 molecules. The product with RT=26.7 min was subjected to mass spectrometry with electron impact ionization. This gave the molecular ion (m/z=400) with major fragment ions 398 (M−2H) and 380 (398−H2O). This product was identified as monohydroxycholecalciferol.

### 20-hydroxycholecalciferol and 20,23-dihydroxycholecalciferol Metabolism by P450scc

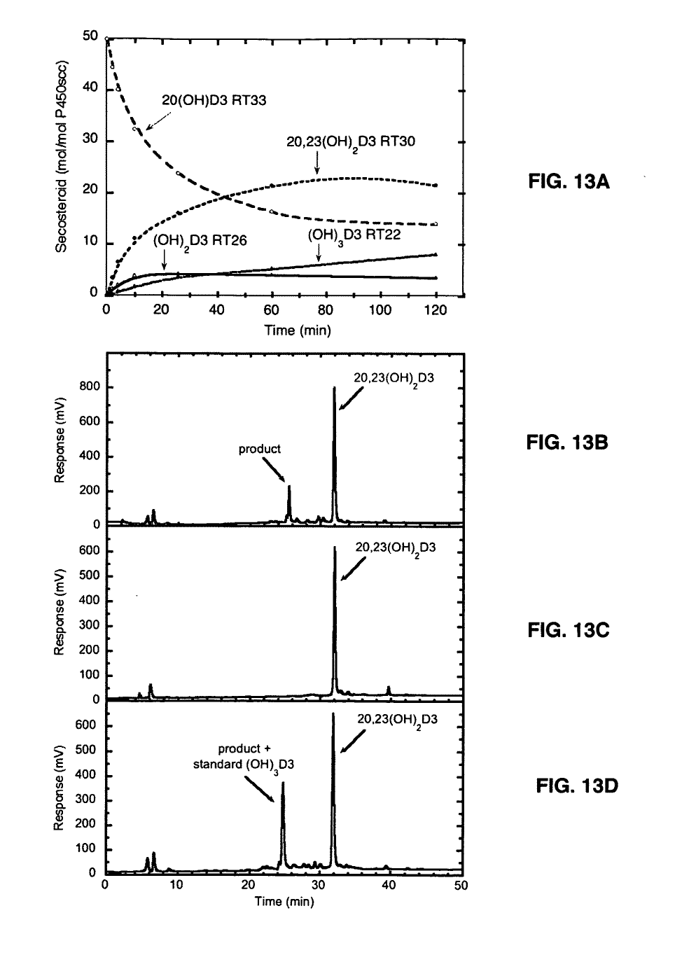

Incubation of 20-hydroxycholecalciferol in cyclodextrin with P450scc resulted in the formation of 20,23-dihydroxycholecalciferol (RT=30 min) and trihydroxycholecalciferol (RT=22 min) (FIG. 13A). A small lag (0-3 min) was seen in the time course for formation of trihydroxycholecalciferol, consistent with accumulation of 20,23-dihydroxycholecalciferol being required before the trihydroxycholecalcifero can be produced. A product with RT=26 min was also observed, as seen for metabolism of cholecalciferol and identified as a dihydroxy derivative (FIGS. 12A-12B). There was no lag in its time course, consistent with it being formed by a single hydroxylation of 20-hydroxycholecalciferol. The products with retention times of 26.7 min and 32 min in FIGS. 12A-12B, identified as monohydroxycholecalciferol derivatives by mass spectrometry (as described above), were not seen as products from 20-hydroxycholecalciferol, as would be expected.

Incubation of 20,23-dihydroxycholecalciferol with cytochrome P450scc in cyclodextrin resulted in one major product with RT=25.5 min, identical to that for trihydroxyvitamin D3 standard added to the test reaction following sample extraction (FIG. 13B-13D). This demonstrates that the trihydroxycholecalciferol can be made from 20,23-dihydroxycholecalciferol and thus provides the sites of two of the three hydroxyl groups added to vitamin D3 by P450scc.

### NMR Identification of dihydroxycholecalciferol as 20,23-dihydroxycholecalciferol

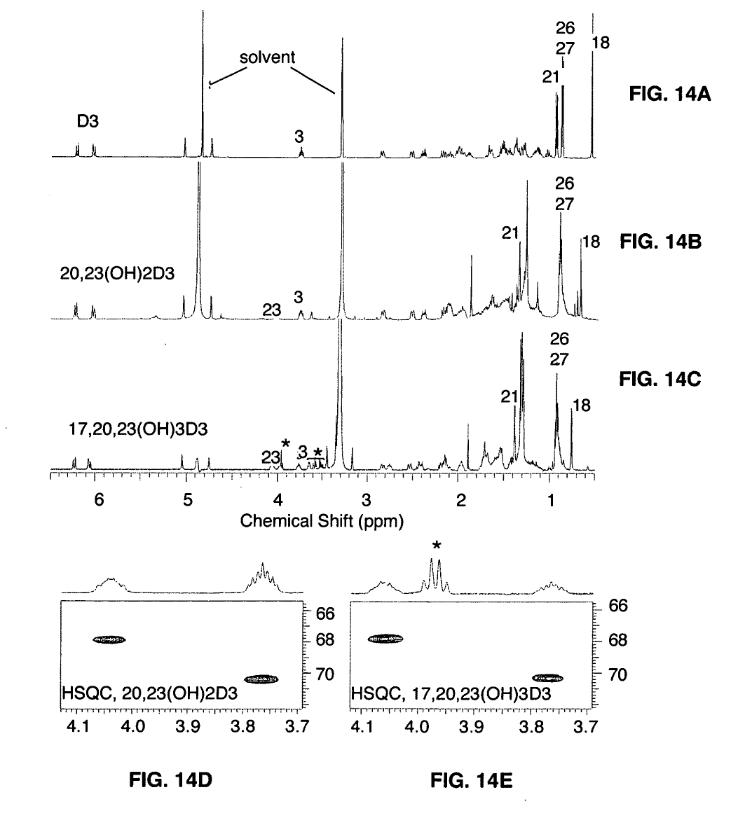

NMR was performed on two preparations of the major dihydroxycholecalciferol metabolite, one synthesized directly from cholecalciferol and the other from the purified intermediate, 20-hydroxycholecalciferol. Both gave essentially identical NMR spectra. Identification of the hydroxylation sites in both dihydroxycholecalciferol and trihydroxycholecalciferol was started by comparing their 1D proton NMR to that of the parent vitamin D3, as shown in FIGS. 14A-14C. For both dihydroxycholecalciferol and trihydroxycholecalciferol, the chemical shifts for 6-CH, 7-CH, 3-CH(OH), 19-CH2 and 9-CH2 were the same as those in cholecalciferol. 21-Me shifted downfield from 0.95 ppm (proton, doublet)/19.54 ppm(carbon) in cholecalciferol to 1.36 ppm (singlet)/26.3 ppm in the dihydroxy metabolite and 1.39 ppm(singlet)/23.2 ppm in the trihydroxy metabolite. This is a classical indication of 20-hydroxylation. Furthermore, compared with vitamin D3, besides 3-CH at 3.76 ppm/70.7 ppm, another hydroxylated CH group at 4.06 ppm/67.9 ppm appears in both metabolites (expansions of HSQC spectra are shown in FIGS. 14D-14E). This clearly indicates that the second hydroxylation occurs on a methylene group, either in the C-ring, D-ring or the side chain.

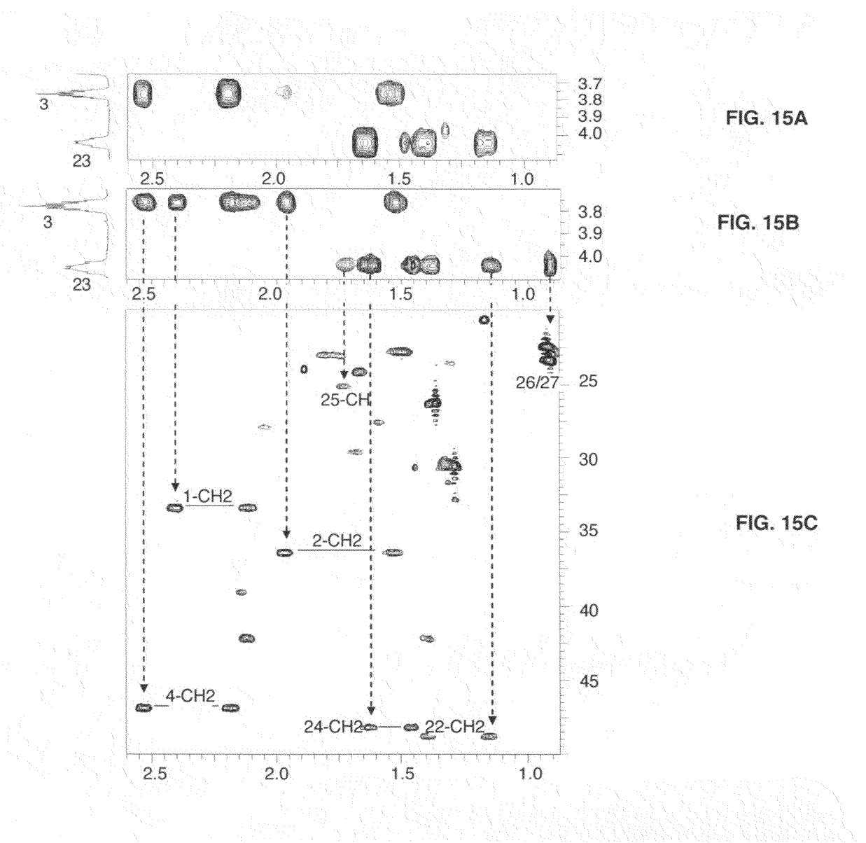

To identify the exact position for the second hydroxylation, 2D COSY, TOCSY and HSQC spectra were analyzed. In the COSY spectrum (FIG. 15A), the correlation between 3-CH and 4-CH2 (2.53 ppm and 2.19 ppm), and the correlation between 3-CH and 2-CH2 (1.97 ppm and 1.52 ppm) are clearly intact. In the TOCSY spectrum (FIG. 15B), all the expected correlations from 3-CH in the A-ring are the same as in the parent cholecalciferol. This further confirms that no hydroxylation occurs in the A-ring. The new hydroxylated CH shows correlations in the COSY spectrum to four protons, analysis of HSQC indicates that these protons belong to two methylene groups (FIG. 15C). The complete spin system revealed by the TOCSY spectrum unambiguously indicates that these two methylene groups are at position 22 and 24, as two methyl groups (C26 and C27) and a methine group (C25) are in this spin network. Therefore, this hydroxylation must be at C23. The TOCSY spectrum for the dihydroxy metabolite also confirms the first hydroxylation is at position 20, since there is no additional correlation assignable to 20-CH (1.36 ppm for proton in parent cholecalciferol). Hydroxylation at position 20 transforms it to a tertiary alcohol that does not participate in the spin system of the side chain.

### NMR Identification of trihydroxyvitamin D3 as 17α,20,23-trihydroxycholecalciferol

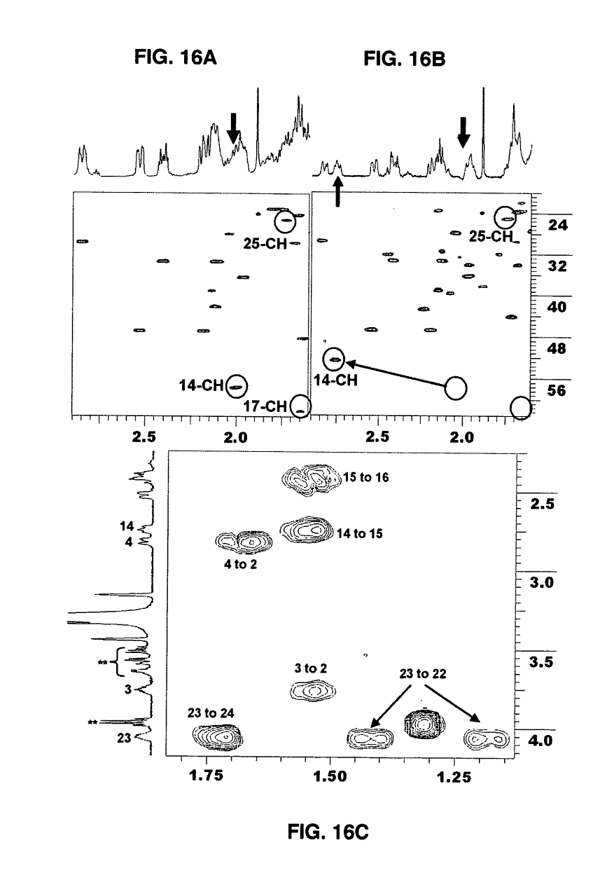

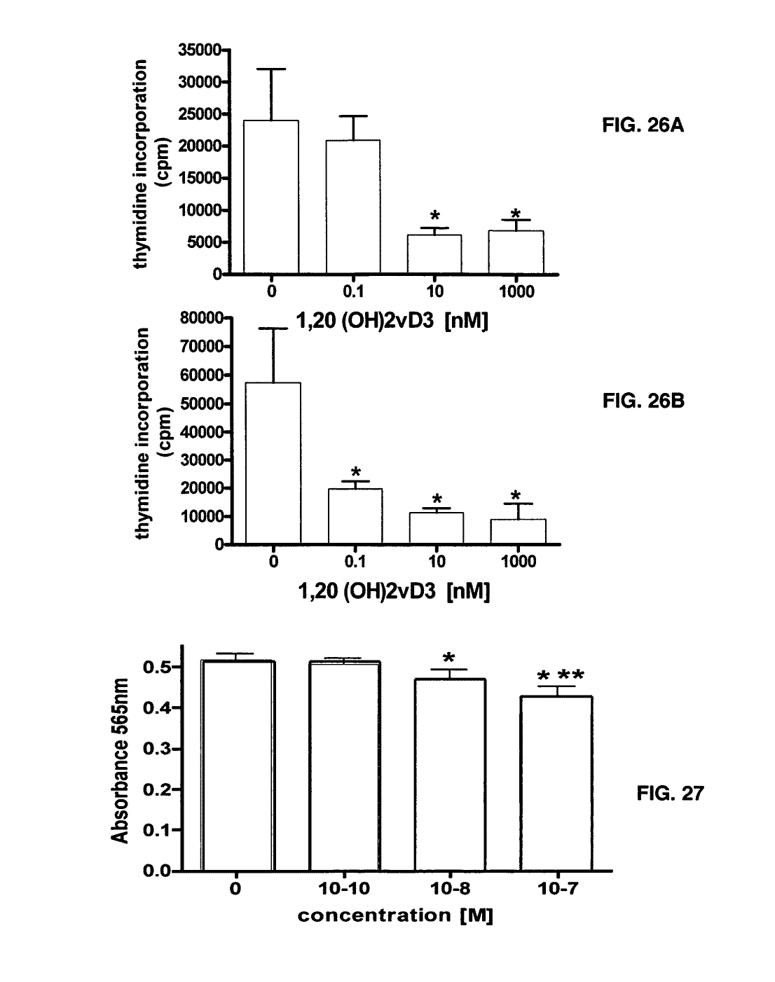

The NMR analysis is shown in FIG. 16A. A critical difference in the proton 1D NMR is the appearance of a triplet peak at 2.75 ppm (FIG. 16B). HSQC indicates that this is from is a methine group. Besides 3-CH and 23-CH, no additional CH bearing a hydroxyl group is present, ruling out possible hydroxylation at methylene groups. Since all four methyl groups are accounted for, the third hydroxyl group must exist as a tertiary alcohol from hydroxylation of a methine group. The candidates are 14-CH (2.0 ppm/57.7 ppm), 17-CH (1.64 ppm/62.0 ppm) and 25-CH (1.74 ppm/25.2 ppm), with their proton/carbon chemical shifts for the dihydroxy precursor indicated in parenthesis. Analysis of the 2D HSQC NMR clearly indicates that 25-CH is intact. Additional evidence include: 1) there are virtually no changes in chemical shifts for 24-CH2 (FIG. 16C) and 26/27-CH3 (FIGS. 14A-14C); and 2) hydroxylation at 25-CH is unlikely to cause 14 or 17 downshift to 2.75 ppm due to its remoteness. The third hydroxyl group is therefore in position 14 or 17.

The only COSY correlation detected from 2.75 ppm is to a 15-CH2 group at 1.53/21.9 ppm. Further analysis indicates that the 16-CH2 signals have shifted to 1.80 and 2.44 ppm (protons) and 32.0 ppm (carbon), as indicated by the COSY correlation between 1.53 ppm (15-CH2) to 2.44 ppm (one proton on 16-CH2). This strongly suggests that third hydroxylation occurs at position 17. Consistent with this assignment, the proton chemical shifts for both 18-Me (0.69 to 0.75) and 21-Me (1.36 to 1.39) have shifted downfield slightly. Finally, it was not possible to collect a workable HMBC spectrum which in theory should have unambiguously indicated the third hydroxylation position, due to the limited amount of trihydroxycholecalciferol available. Despite this, analysis of all the spectra collectively indicate that this trihydroxy metabolite is 17α,20,23-trihydroxycholecalciferol.

### Metabolism of 1α-hydroxycholecalciferol by P450scc

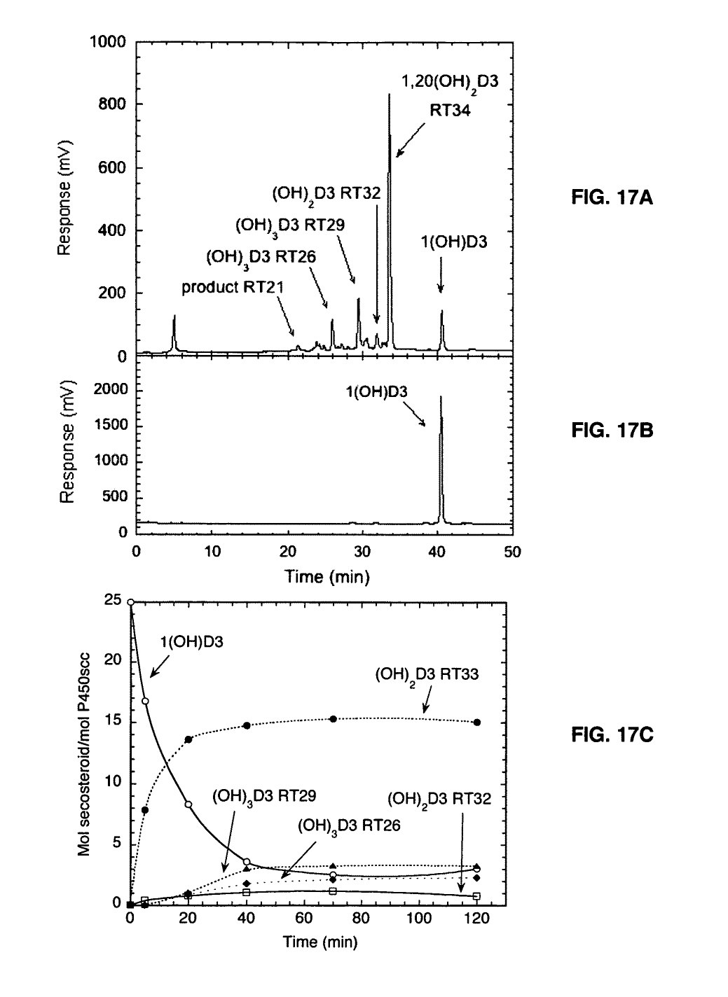

Incubation of 1α-hydroxycholecalciferol dissolved in 0.45% cyclodextrin, with P450scc, resulted in one major product and several minor ones (FIGS. 17A-17B). Four of the products were purified and subjected to mass spectral analysis with ESI. The major product (RT=33 min) gave the most abundant ion at m/z=439.7 (416.7+Na⁺) from which the sample was identified as dihydroxy vitamin D3 derivative. A major ion was also observed at 856.4 corresponding to Na⁺ complexed to two trihydroxy vitamin D3 derivative molecules. The electrospray mass specta for the products with RT=29 min and RT=26 min in FIGS. 1A-1B were similar, with m/z=455 (432+Na⁺) for the major ion. These products were therefore identified as trihydroxy vitamin D3 derivatives. A major ion was also observed at m/z=887 for both, which corresponds to Na⁺ complexed to two trihydroxy vitamin D3 derivative molecules. The electrospray mass spectrum for the product with RT=32 min in FIG. 12A-12D had m/z=439 (416+Na⁺) and was identified as another dihydroxy cholecalciferol derivative.

A time course for the metabolism of 1α-hydroxycholecalciferol in cyclodextrin is shown in FIG. 17C. The two monohydroxy products (RT=32 min and RT=33 min) appeared without a lag, consistent with them requiring only one P450scc-catalysed hydroxylation for their formation. The two trihydroxy products (RT=26 and 29 min) showed an initial lag before they appeared, suggesting that some accumulation of a dihydroxy product was required to serve as their substrate. The product with RT=21 min in FIGS. 16A-16B was not produced in sufficient quantities for reliable quantitation and was not included in the time course.

### P450scc Metabolic Pathway and Metabolite Structures

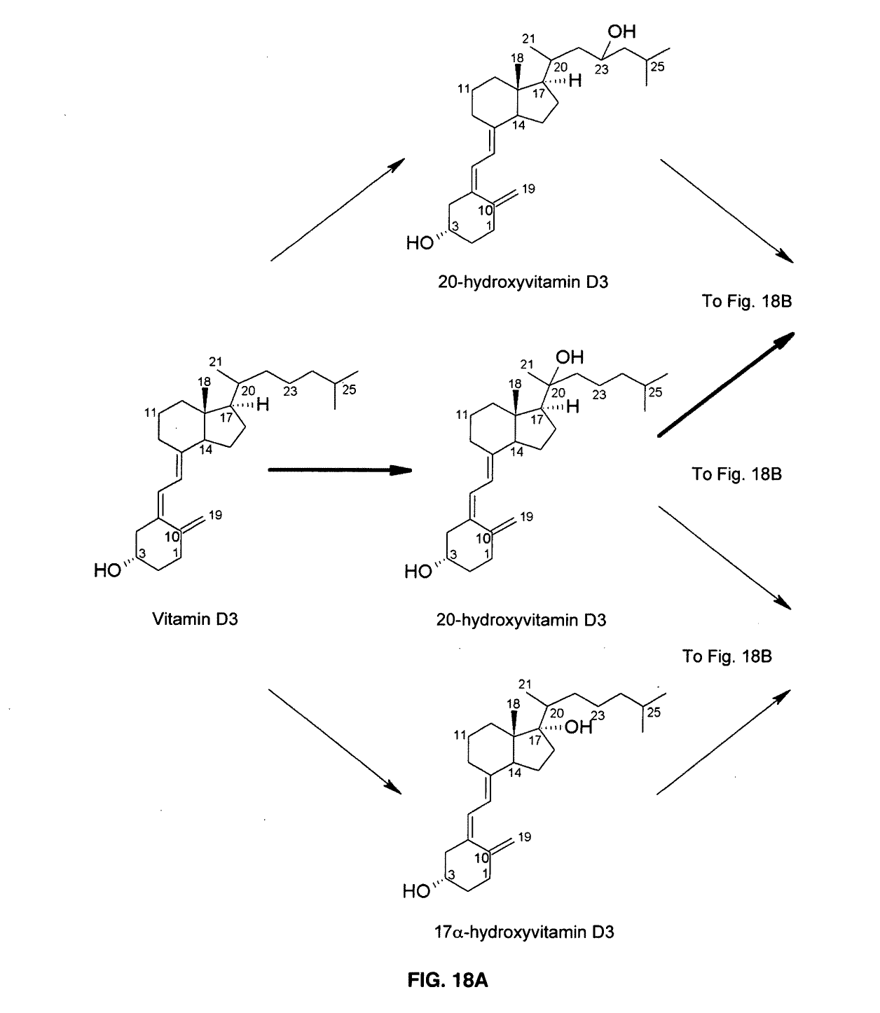

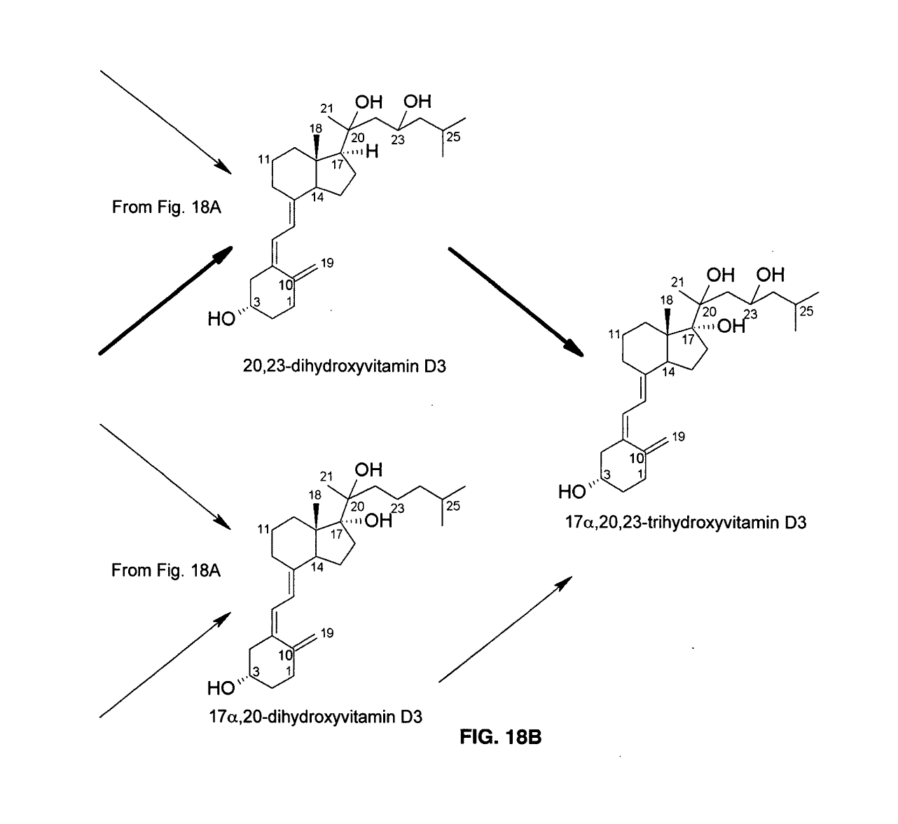

The six products seen for metabolism of vitamin D3 by P450scc in this study can be explained by the various possible combinations of the three hydroxylations that have been identified (FIG. 18). All products and/or intermediates can ultimately be converted to 17α,20,23-trihydroxycholecalciferol. Thus, minor products could be identified with NMR structures of only 20-hydroxyvitamin D3, 20,23-dihydroxycholecalciferol and 17α,20,23-trihydroxycholecalciferol. The major pathway of vitamin D3 metabolism by cytochrome P450scc involves initial hydroxylation at C20 followed by hydroxylations at C23 and C17, respectively (pathway in bold). There are additional minor pathways where the relative order of the three hydroxylations differ.

### EXAMPLE 5

### 20-hydroxycholecalciferol Activity

### Proliferation of Epidermal Keratinocytes is Inhibited

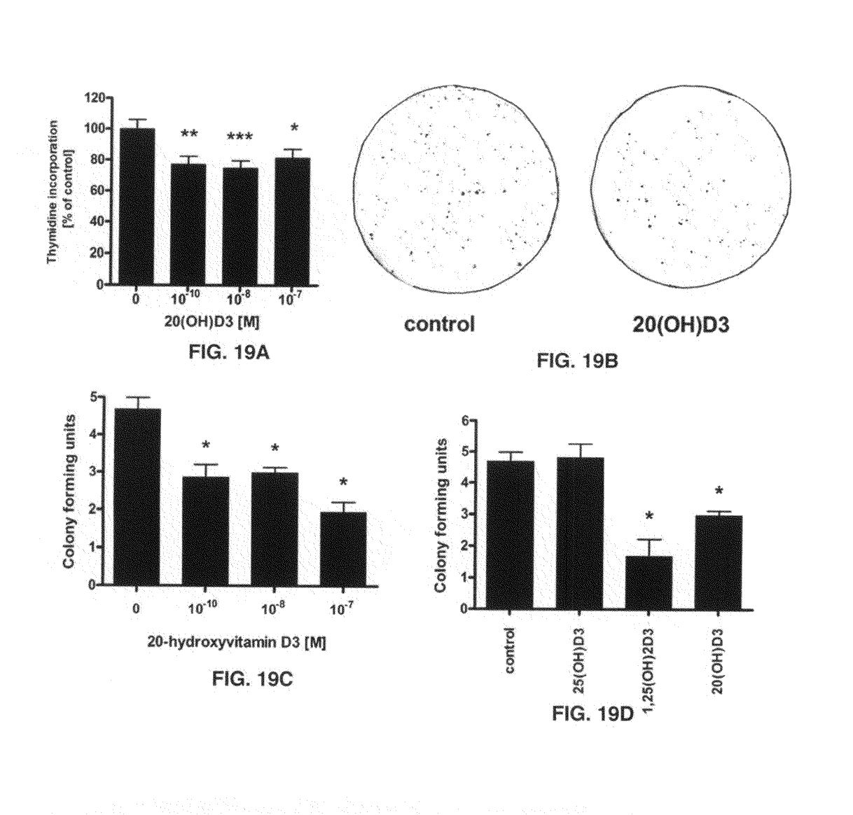

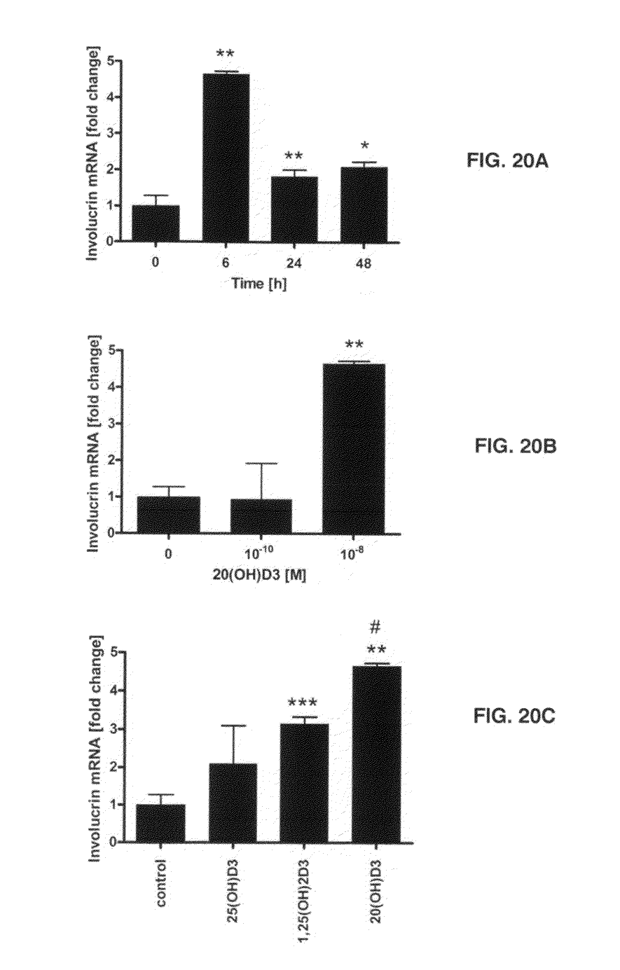

HaCaT keratinocytes were incubated for 48 hours in DMEM medium. DNA synthesis was then measured with a [³H]-thymidine assay. As shown in FIG. 19A, 20-hydroxycholecalciferol inhibited DNA synthesis at concentrations of 10⁻⁸ and at 10⁻⁷ M. To further define the antiproliferative effect of the ligands, HaCaT keratinocytes were incubated for 10 days in DMEM in the presence or absence of vitamin D3 hydroxy-derivatives and colony forming potential was measured. As shown in FIGS. 19B-19C, 20-hydroxycholecalciferol inhibited colony formation by HaCaT cells. 20(OH)D3 at 10⁻⁸ M inhibited colony formation by 36% and at 10⁻⁷ M by 58%. 1α,25-dihydroxycholecalciferol at 10⁻⁸ M inhibited colony formation by 64% while 25-hydroxycholecalciferol had no significant effect (FIG. 19D). Thus, 20-hydroxycholecalciferol shows antiproliferative potency comparable to but lower than 1α,25-dihydroxycholecalciferol.

The effect of 20-hydroxycholecalciferol was tested on normal epidermal keratinocytes using the technique of flow cytometry. The cells were seeded into Petri dishes and, after 48 h of treatment with 20-hydroxycholecalciferol or vehicle, were collected, fixed, stained with propidium iodide and submitted for flow cytometric analysis. Control cells were distributed as follows: 37±10% in G1/0, 38±14% in S and 25±6% in G2/M phase of the cell cycle (n=3). Treatment of cells for 24 h with 10 nM 1α,25-dihydroxycholecalciferol resulted in significant G1/0 (52±2%, P<0.05) and G2/M 35±5%, P<0.05) arrests (S phase: 13±7%, P<0.05). Similarly, treatment of cells for 48 h with 10 nM 20(OH)D3 resulted in G1/0 (52±5%) and G2/M (35±8%, P<0.05) arrests (S phase: 13±12%, p<0.05).

### Expression of Genes Involved in Keratinocyte Differentiation are Affected

The action of 20-hydroxycholecalciferol was compared with that of 1α,25-dihydroxycholecalciferol on the expression of involucrin and cytokeratin 14 genes in normal epidermal keratinocytes. 20-hydroxycholecalciferol inhibited expression of cytokeratin 14 and stimulated expression of involucrin in a dose- and time-dependent fashion. 20-hydroxycholecalciferol at 10⁻¹⁰ M inhibited expression of cytokeratin 14 mRNA. The effect was maximal 1 h after treatment, e.g. decreased to 45% of the control value, and started to fade at 6 h reaching 62% of the control. The inhibitory effect was significant at both 10⁻¹⁰ and 10⁻⁸ M concentrations. Of note, 20-hydroxycholecalciferol showed a significantly higher inhibitory effect on cytokeratin 14 mRNA expression than the 1α,25-dihydroxycholecalciferol. 20-hydroxycholecalciferol (at 10⁻⁸ but not at 10⁻¹⁰ M) stimulated expression of involucrin mRNA. The effect was maximal at 6 h where 4.7-fold stimulation was observed and started to fade by 24 h when stimulation was only 1.8-fold (FIG. 20A). Again, 20-hydroxycholecalciferol has higher potency in inhibiting involucrin mRNA expression in comparison to 1α,25-dihydroxycholecalciferol (˜4.7-fold stimulation vs ˜3.1-fold, P<0.05) (FIG. 20B). 25-hydroxycholecalciferol increased expression of involucrin ˜2.1-fold, however, the effect was statistically insignificant (FIG. 20C).

### Involucrin Expression is Stimulated and Keratinocyte Size and Granularity Increase

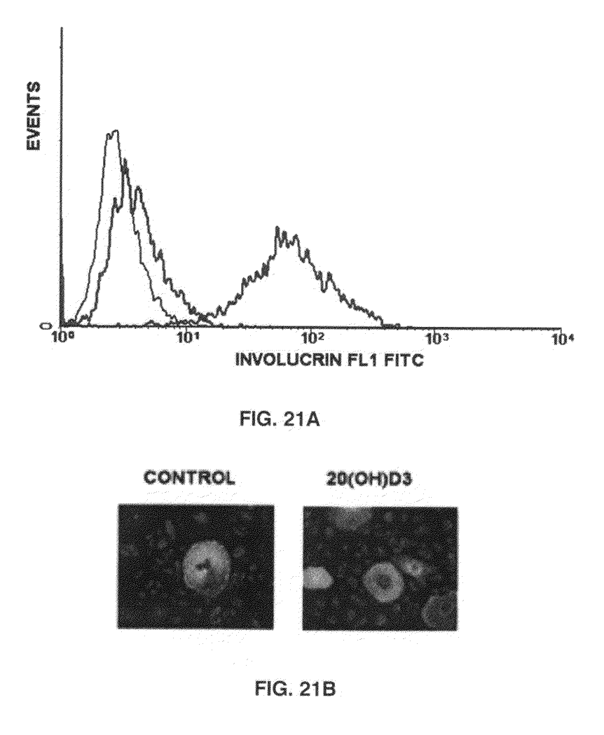

Having established that 20-hydroxycholecalciferol acts at the transcriptional level, it was determined if the changes in the gene expression were reflected in the keratinocyte differentiated phenotype. Expression of involucrin was measured using both flow cytometry and fluorescent microscopy. As shown in FIGS. 21A-21B, treatment of HaCaT keratinocytes with 20-hydroxycholecalciferol at 10⁻⁸ M for 24 h resulted in a ˜3.7 fold increase in the expression of involucrin (control dMFI 19±9, treatment dMFI: 71±8, n=4, P<0.05). Interestingly, effects of 1α,25-dihydroxycholecalciferol and 25-hydroxycholecalciferol were negligible. Moreover, the effects of 20-hydroxycholecalciferol on forward and side scatter of cells were measured. These parameters reflect cell size and granularity, respectively. Both parameters are known to increase during keratinocyte differentiation.

Mean signal intensity (MSI) of forward scatter in control cells was 235±7 and of side scatter was 175±7 (n=3). 20-hydroxycholecalciferol at 0.1 nM significantly increased both forward (MSI: 251±2, P<0.05) and side scatter of HaCaT keratinocytes (MSI: 210±6 P<0.05). 1α,25-dihydroxycholecalciferol acted similarly but only the effect on side scatter was statistically significant (MSI: 219±4, P<0.05). 25-hydroxycholecalciferol also increased both parameters, although only the effect on forward scatter was statistically significant (MSI: 254±0.06, p<0.05). This demonstrates for the first time that 20-hydroxycholecalciferol has similar effects on programmed keratinocyte differentiation to 1α,25-dihydroxycholecalciferol and also has comparable potency.

### 20-hydroxycholecalciferol Inhibits Expression of CYP27B1 and CYP27A1 Genes

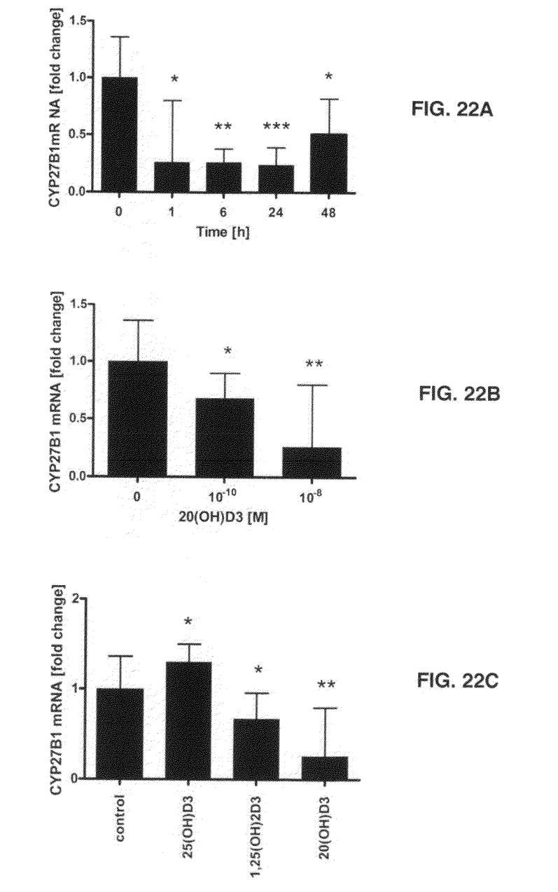

Since expression of CYP27B1 and CYP27A1 genes is inhibited by 1α,25-dihydroxycholecalciferol in the kidney and liver, respectively (11,15) the action of 20-hydroxycholecalciferol was compared with that of 1α,25-dihydroxycholecalciferol on the expression of these genes in normal epidermal keratinocytes. 20-hydroxycholecalciferol inhibited expression of CYP27B1 and CYP27A1 in a dose- and time-dependent fashion (FIGS. 22A-22B). 20-hydroxycholecalciferol at 10⁻⁸ M inhibited expression of CYP27B1-4-fold. The effect was detected at 1 h after treatment, maintained thereafter, and faded at 48 h when inhibition decreased to ˜2-fold (FIG. 16A). Interestingly, 20-hydroxycholecalciferol showed slightly higher potency than 1α,25-dihydroxycholecalciferol on CYP27B1 expression (e.g., 1α,25-dihydroxycholecalciferol at 10⁻⁸ M inhibited gene expression by ˜1.5-fold) (FIG. 22C). In contrast, 25-hydroxycholecalciferol increased expression of CYP27B1 ˜1.3-fold.

20-hydroxycholecalciferol at 10⁻⁸ M inhibited expression of CYP27A1 gradually. The effect was observable at 1 h after treatment with maximum inhibition of ˜3.5-fold occurring at 24 h. The effect started to fade at 48 h when inhibition decreased to ˜2.6-fold. Similarly to the effect on CYP27B1, the effect of 20-hydroxycholecalciferol on CYP27A1 expression was also higher than the effect of 1α,25-dihydroxycholecalciferol (˜2.9-fold at 24 h with 10⁻⁸ M 1α,25-dihydroxycholecalciferol). 25-hydroxycholecalciferol also decreased expression of CYP27A1 ˜1.9-fold. These results confirm the general actions reported for 1α,25-dihydroxycholecalciferol and 25-hydroxycholecalciferol on CYP27B1 and CYP27A1 (11,13,15) using adult human epidermal keratinocytes. and demonstrate for the first time that 20-hydroxycholecalciferol can act as a potent inhibitor of both genes involved in 1α,25-dihydroxycholecalciferol synthesis.

### 20-hydroxycholecalciferol has Significantly Lower Potency on CYP24 Transcription than 25-hydroxyvitamin D3 or 1α,25-dihydroxyvitamin D3

The action of 20-hydroxycholecalciferol was compared with the actions of 1α,25-dihydroxycholecalciferol and 25-hydroxycholecalciferol on the transcriptional activity of the CYP24 promoter in normal epidermal keratinocytes. Normal epidermal keratinocytes were transfected with either a luciferase reporter construct driven by CYP24 promoter or a promoterless luciferase construct. Transcriptional activity of the CYP24 promoter was stimulated ˜21-fold, ˜12-fold and only ˜2.5-fold by 1α,25-dihydroxycholecalciferol, 25-hydroxycholecalciferol, and 20-hydroxycholecalciferol, respectively. None of these substrates affected the activity of the promoterless (pLuc) construct.

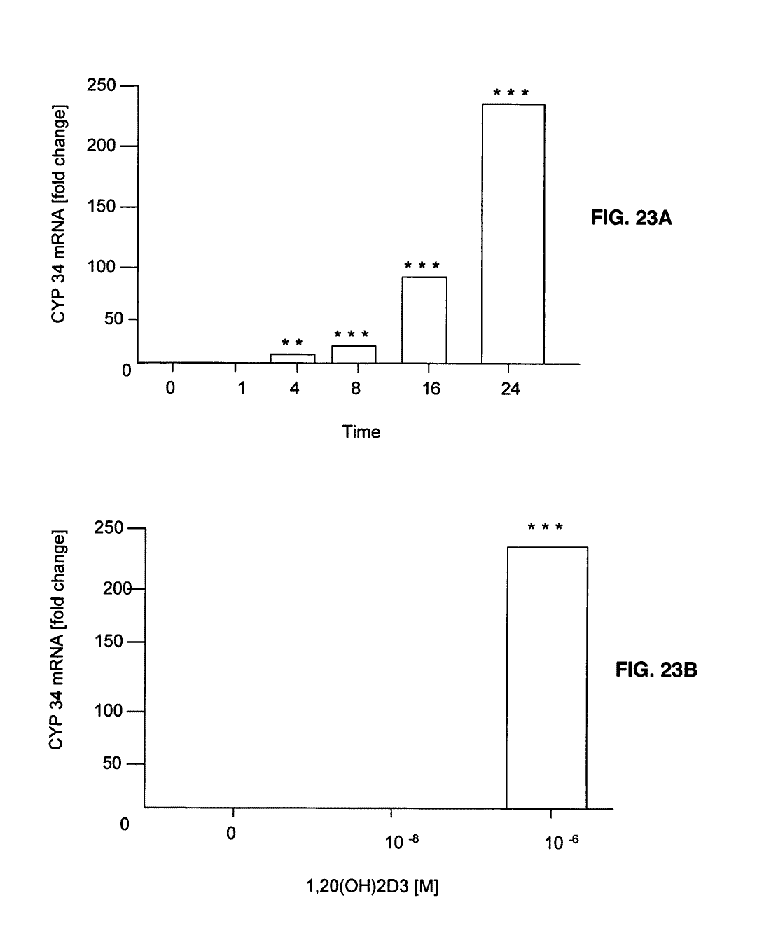

The effect of 20-hydroxycholecalciferol on the expression of CYP24 mRNA was tested. As shown in FIGS. 23A-23B, 10 nM 20-hydroxycholecalciferol at 24 h increased CYP24 mRNA levels only 1.3-fold but at 10⁻⁶ ˜247-fold stimulation was observed. Effect on CYP24 was thus significantly weaker since it required much higher concentration of 20-hydroxycholecalciferol. Active form of vitamin D3 (1α,25-dihydroxycholecalciferol) stimulates expression of CYP24 in kidney, in cultured human neonatal keratinocytes and in other skin and non-skin cells. These findings are confirmed herein and show for the first time that 20-hydroxycholecalciferol affects CYP24 transcriptional activity to a significantly lower degree in adult epidermal keratinocytes as compared to the potency of 1α,25-dihydroxycholecalciferol and 25-hydroxycholecalciferol. In regards to the effect at 10 nM, slight discrepancy between results obtained with reporter assay and real-time PCR can be explained by higher sensitivity of promoter-driven construct in the system. In separate experiments using HaCaT keratinocytes, 20-hydroxycholecalciferol failed to stimulate the CYP24 promoter activity, which indicates that the stimulatory effect depends on the type of cells used for the experiment.

It is contemplated that this novel compound, 20-hydroxycholecalciferol, may play a minor role in regulating the inactivation of the active forms of vitamin D3, which is in opposition to its inhibitory actions on expression of CYP27A1 and CYP27B1 genes (see above). The discrepancy between typical set of responses to 1α,25-dihydroxycholecalciferol and to 20-hydroxycholecalciferol can be explained by different conformations of ligand-receptor complex eliciting different cellular responses. This biological mechanism has been documented in case of PPAR gamma receptor and its different ligands. Furthermore 20-hydroxycholecalciferol can be metabolized to different compounds, including di- and tri-hydroxy-vitamin D3 that potentially may interact with the receptor.

### 20-hydroxyvitamin D3 Stimulates VDRE Through VDR in HaCaT Keratinocytes

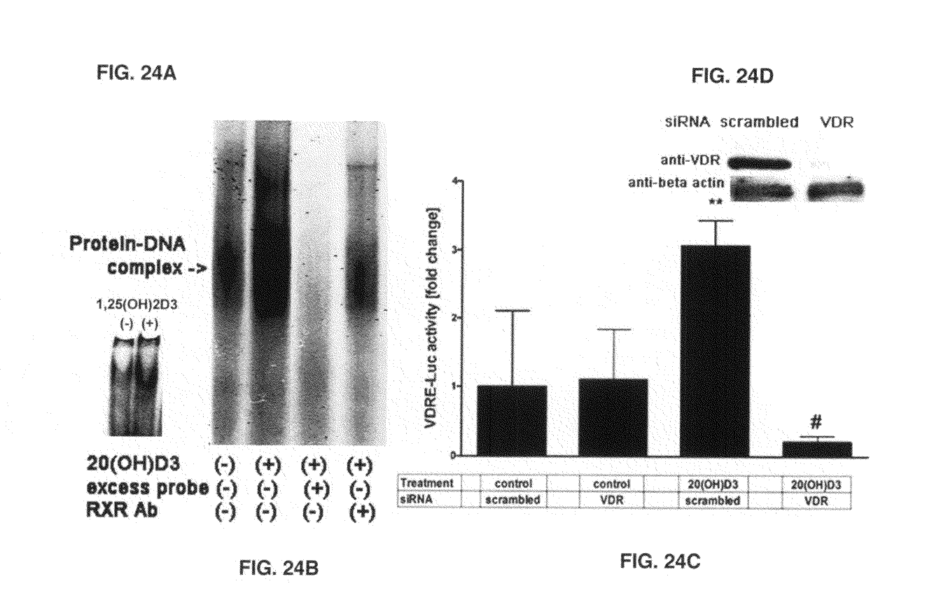

HacaT keratinocytes were stimulated for 24 h with 10 nM 20-hydroxycholecalciferol, then nuclear extracts were prepared and incubated with labeled VDRE probe. As shown in FIG. 24A, 20-hydroxycholecalciferol stimulated binding activity of protein complexes to VDRE probe. The binding was specific since excess of unlabelled VDRE caused complete disappearance of the signal. Moreover, addition of RXR antibody resulted in significant decrease in the signal, which evidences that RXR protein is part of 20-hydroxycholecalciferol-stimulated protein complex that binds to VDRE. As shown in the FIG. 24B, positive control, 1α,25-dihydroxyvitamin D3 stimulated binding of protein complexes to VDRE.

Cells were transfected with VDRE-Luc and with scrambled or VDR siRNA. As shown in the inset of FIG. 24D, VDR siRNA caused almost complete disappearance of VDR expression on protein level 24 h after transfection. Keratinocytes were then incubated with 10 nM 20-hydroxycholecalciferol or vehicle control for 24 hours. As shown on FIG. 24C, transfection of HaCaT keratinocytes with VDR siRNA had no effect on basal VDRE-driven transcriptional activity (cells transfected with scrambled siRNA). Treatment of cells transfected with scrambled siRNA with 10 nM 20-hydroxycholecalciferol resulted in ˜3-fold increase in VDRE-driven luciferase activity (p<0.005, versus control).

Transfection of keratinocytes with VDR siRNA decreased the 20-hydroxycholecalciferol-stimulated VDRE activity (p<0.00005). Of note, there was no statistical significance in transcriptional activity between cells transfected with scrambled siRNA and treated with vehicle and cells transfected with VDR siRNA and treated with 20-hydroxycholecalciferol. Above data indicate that 20-hydroxycholecalciferol acts through VDR and VDRE. However, it cannot be excluded that 20-hydroxycholecalciferol activates other receptors including membrane receptors, which may also be suggested by a rapid increase in mRNA levels. For example, 1α,25-dihydroxycholecalciferol can act through several membrane-associated receptors and respective downstream pathways including Annexin II/Phosphatidylinositol 3-kinase/Ras/MEK/Extracellular signal regulated kinase 1/2/ and c-Jun N-terminal kinase 1 pathway in the keratinocytes.

### S or G1/G0Arrest and Apoptosis in Human Cancer Cell Lines

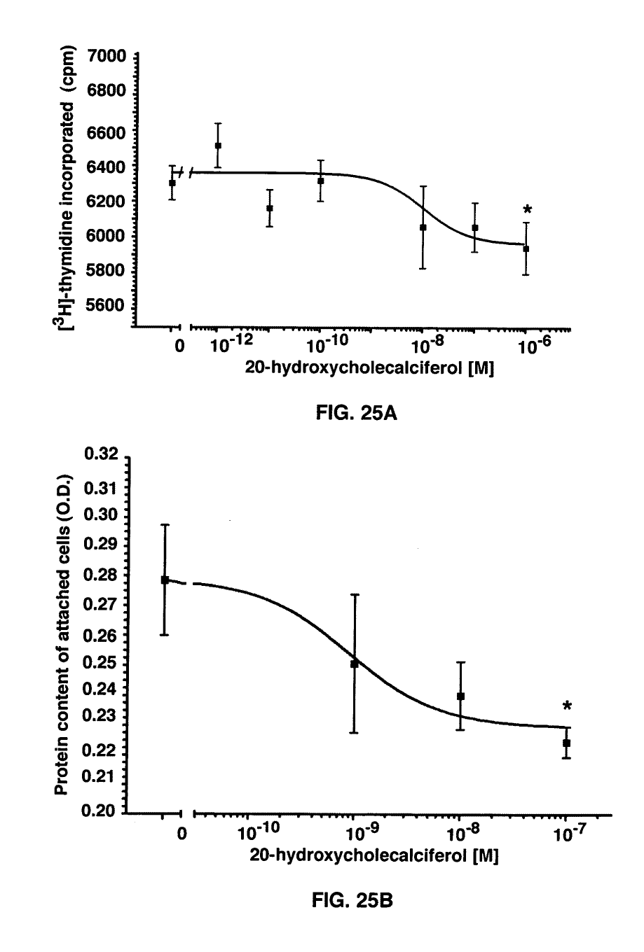

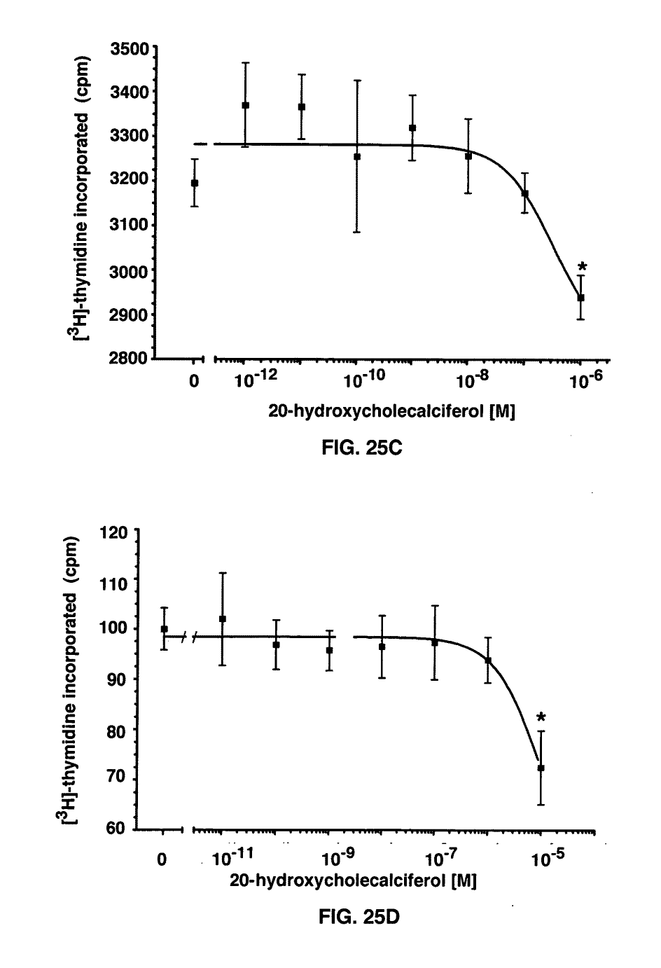

20-hydroxycholecalciferol induces S arrest and apoptosis in human breast carcinoma MD-MBA-231 cells (FIG. 25A), in human osteosarcoma MG-63 cells (FIG. 25B) and in human prostate carcinoma cells PC-3 (FIGS. 25C). 20-hydroxycholecalciferol induces S arrest and apoptosis in human radial growth phase amelanotic melanoma WM35 cells (FIG. 25D). In the presence of 20-hydroxycholecalciferol the EC₅₀ for MD-MBA-231 is 1×10⁻⁸ M, the EC₅₀ for MG-63 is 5.7×10⁻⁷ M and the EC₅₀ for PC-3 is 5.7×10⁻⁷ M. In the presence of 20-hydroxycholecalciferol WM35 cells viability started declining with concentrations greater than about 1×10⁻⁷M.

MD-MBA-231, MG-63, PC-3, and WM164 cells were seeded into Petri dishes and then incubated for 24 h with 20-hydroxycholecalciferol in DMEM medium containing 5% FBS. Then cells were fixed, DNA stained and samples read with flow cytometer as described (7). Data was analyzed with Cell Quest (BD Biosciences). Cell cycle phases were assessed in viable cell populations and subG1 contents were calculated within whole cell population. Results are given in Table 11.

TABLE 11

| | | | Cell cycle phases of | | |
|---|---|---|---|---|---|
| Cell line & | Viable | | viable cells | | |
| Compound | Cells | subG1 | G0/G1 | S | G2/M |
| MD-MBA-231 | | | | | |
| Control | 48 | 52 | 81% | 7% | 12 |
| 20(OH)D3 | 8 | 92 | 47% | 43% | 10 |
| MG-63 | | | | | |
| Control | 67 | 33 | 63% | 20% | 17 |
| 20(OH)D3 | 18 | 82 | 52% | 33% | 15 |
| PC-3 | | | | | |
| Control | 77 | 23 | 53% | 23% | 24 |
| 20(OH)D3 | 4 | 96 | 24% | 76% | 0 |
| WM164 | | | | | |
| Control | 98 | 2 | 63% | 27% | 10 |
| 20(OH)D3 | 87 | 13 | 70% | 24% | 7 |

### EXAMPLE 6

### 1α,20-dihydroxycholecalciferol Activity

### Real-Time RT PCR

The RNA from HaCaT keratinocytes treated with 1α,20-dihydroxycholecalciferol was isolated and RT PCR run as described in Example 1.

### Treating Cells with 1α,20-dihydroxycholecalciferol and [³H]-thymidine Incorporation

HaCaT keratinocytes were plated out in 24-well plates, 50,000 cells/well. Test compounds, 1α,20-dihydroxycholecalciferol and 1α,25-dihydroxycholecalciferol, were diluted from ethanol stocks into DMEM medium containing 5% charcoal-treated serum and added to an overnight culture of the cells to a final concentration of either 10⁻⁸ M or 10⁻¹⁰ M. The final concentration of ethanol vehicle was 10⁻⁶ M. After 20 and 44 h of incubation, [³H]-thymidine (specific activity 88.0 Ci/mmol; Amersham Biosciences, Picataway, N.Y., USA) was added at the concentration of 1.0 μCi/ml medium. After 4 h media were discarded, cells washed with cold phosphate-buffered saline and incubated in 10% trichloroacetic acid for 30 min. Cells were washed again with phosphate-buffered saline, 100 μl 1.0 M NaOH was added to each well and plates incubated for 30 min at 30° C. The supernatant was collected and the ³H-radioactivity measured by scintillation counting using a Direct Beta-Counter Matrix 9600 (Packard). The [³H]-thymidine incorporation into DNA was measured separately for each well and the results entered into the calculation as the mean of 6 wells for each condition in a series of six experiments (n=36). Data were analyzed with GraphPad Prizm Version 4.0 (GraphPad Software Inc., San Diego, Calif., USA) using t tests. Differences were considered significant when p<0.05.

### Cytotoxicity

Cells are treated with the test compound, washed, fixed and stained with the Sulphorhodamine B dye (SRB). The incorporated dye is then liberated from the cells in a tris-base solution. An increase or decrease in the number of cells (total biomass) results in a concomitant change in the amount of dye incorporated by the cells in the culture. Cells were seeded in growth medium at 10,000 per well in 96-well plates. After 12 h of culture the medium was changed to 5% charcoal-treated serum and cells cultured for a further 47 h with serial dilutions of 1α,20-dihydroxycholecalciferol (diluted as for as for thymidine incorporation). Acetic acid was then added to a final concentration of 20% from a 50% stock and cells incubated for 1 h. Cells were stained with SRB 0.4% (Sigma), washed with 1% acetic acid and dried. Trsi-HCl was added and the absorbance measured at 565 nm. The absorbance of blank medium only, was also measured also at 690 nm.

### Effects of 1α,20-dihydroxycholecalciferol on Keratinocyte Proliferation

Treatment of HaCaT keratinocytes with 1α,20-dihydroxycholecalciferol led to suppression of [³H]-thymidine incorporation into the DNA in a concentration dependent manner compared to the control which contained the ethanol vehicle (FIGS. 26A-26B). Concentrations of 0.1 nM and 10 nM were chosen for further testing the effects of 1α,20-dihydroxycholecalciferol on proliferation of keratinocytes and comparison to the effects of 1α,25-dihydroxycholecalciferol. The 1α,20-dihydroxycholecalciferol decreased DNA synthesis by 30% at a concentration of 0.1 nM and by 50% at 10 nM. These differences from the control were statistically significant (p<0.05). A similar decrease in DNA synthesis was seen following treatment of cells with equivalent concentrations of 1α,25-dihydroxycholecalciferol. There was no statistical significance between the results for treatments with 1α,20-dihydroxycholecalciferol and 1α,25-dihydroxycholecalciferol.

In vitro detection of any toxic effect of 1α,20-dihydroxycholecalciferol was determined using sulforhodamine B assay system which measures total biomass by staining cellular proteins with sulforhodamine B dye. As shown in FIG. 27, 1α,20-dihydroxycholecalciferol caused a decrease in number of viable cells and this effect was dose dependant.

### Effects on Expression of CYP24 mRNA in Keratinocytes

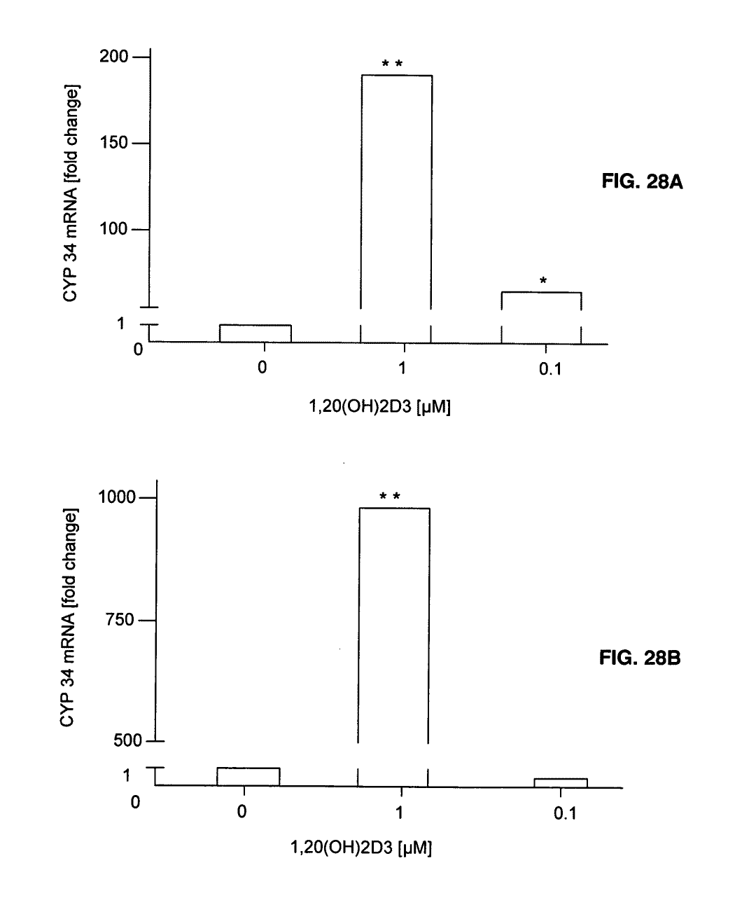

Since CYP24 is an important physiological target of 1α,25-dihydroxycholecalciferol in the kidney and peripheral tissues, including skin, the action of 1α,20-dihydroxycholecalciferol was tested on the CYP24 mRNA level in HaCaT keratinocytes. HaCaT keratinocytes were treated with 1α,20-dihydroxycholecalciferol at different concentrations for 6 h and 24 h. As shown in FIGS. 28A-28B, 1α,20-dihydroxycholecalciferol at a concentration of 1 μM markedly increased the Cyp 24 mRNA level. Modest stimulation was seen with 0.1 μM 1α,20-dihydroxycholecalciferol following 6 h of treatment but the effect was lost by 24 h. 1α,20-dihydroxycholecalciferol indeed exerts biological activity, although to much lesser degree than 1α,25-dihydroxycholecalciferol.

### EXAMPLE 7

### Biological Activity of 20,23-dihydroxycholecalciferol vs 1α,25-dihydroxycholecalciferol

### Proliferation of Keratinocytes is Inhibited

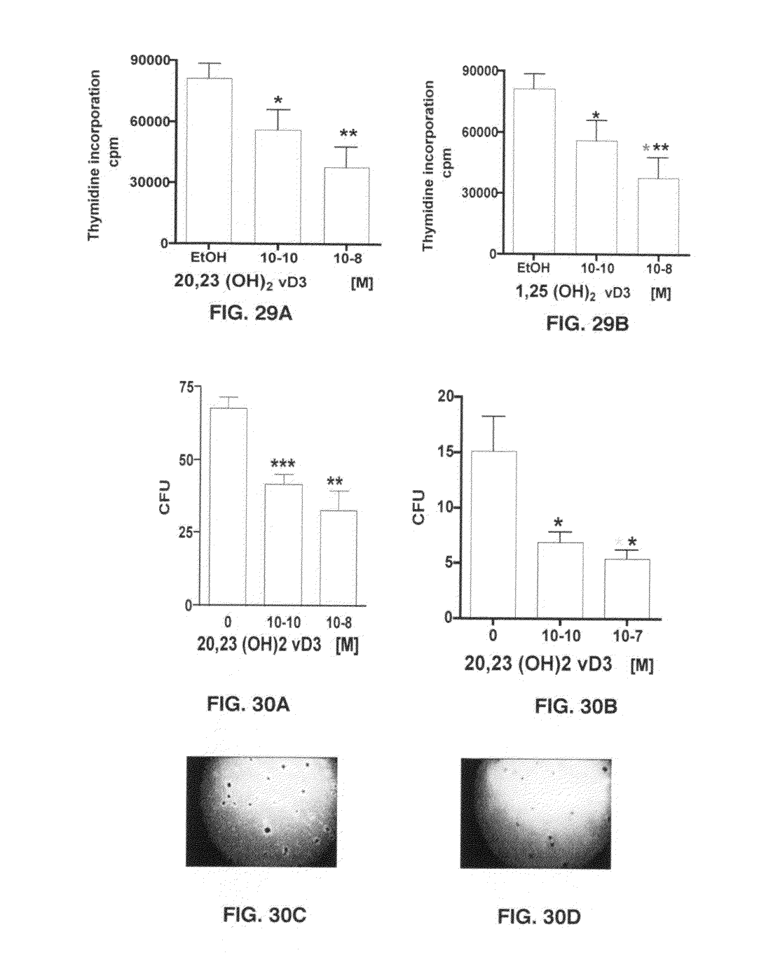

Treatment of keratinocytes with 20,23-dihydroxycholecalciferol (FIG. 29A) led to suppression of [³H]-thymidine incorporation in concentration dependent manner compared to control (ethanol treated, FIG. 29B). Concentrations of 0.1 nM and 10 nM were chosen for further testing of 20,23-dihydroxycholecalciferol on proliferation of keratinocytes compared to 1α,25-dihydroxycholecalciferol. Decrease in DNA synthesis by 30% at 0.1 nM concentration and by approximately 50% at 10 nM concentration has been observed in treatment with 20,23-dihydroxycholecalciferol, which is the same activity compared to 1α,25-dihydroxycholecalciferol. Difference was statistically significant (p<0.05). Decrease in DNA synthesis has been observed in cells treated with 20,23-dihydroxycholecalciferol and 1α,25-dihydroxycholecalciferol.

### Proliferation of Melanoma Cells is Inhibited

Treatment of melanoma cells human SK Mel 188 (FIG. 30A) and hamster AbC1 (FIG. 30B) with 20,23-dihydroxycholecalciferol led to suppression of cell survival in forced suspension culture. This was determined by assessing colony formation of melanoma cells in soft agar. As compared with control (ethanol treated cells; FIG. 30C), 20,23-dihydroxycholecalciferol treatments (FIG. 30D) during suspension decreased both the number and size of colonies. Decrease by 30% at 0.1 nM concentration and by approximately 50% at 10 nM concentration has been observed in AbC1 cells treated with 20,23-dihydroxycholecalciferol. Approximately 50% less colonies bigger than 0.5 mm appeared in Sk Mel 188 melanoma treated with 0.1 nM and 100 nM concentrated 20,23-dihydroxycholecalciferol. Differences were statistically significant (p<0.05).

### HaCaT Cells are Arrested at G0/G1 and G2/M Cell Cycle Phase

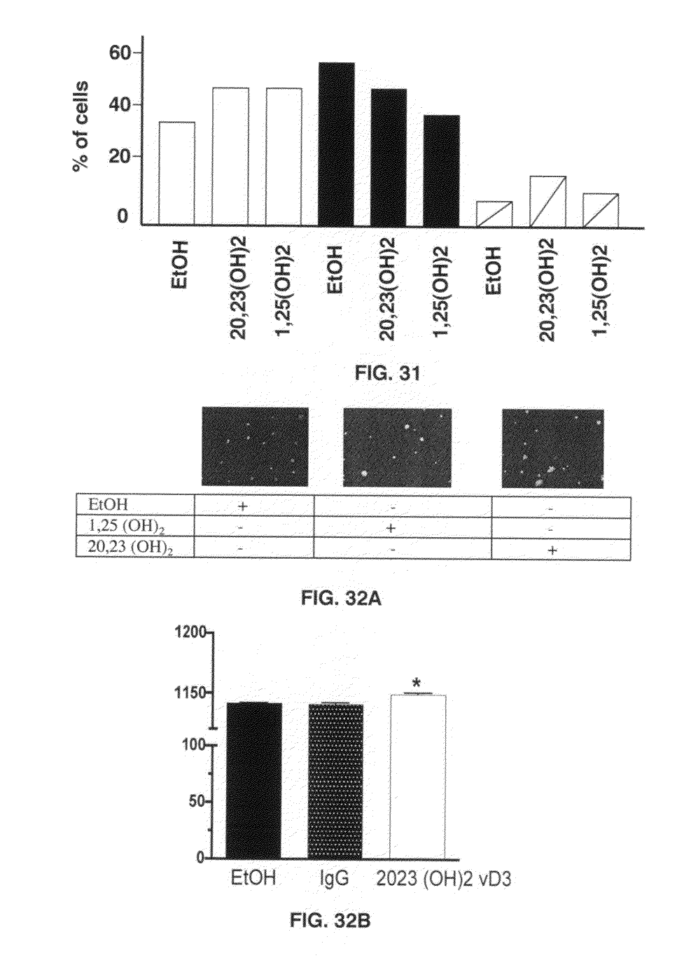

Cells were treated for 24 h with 20,23-dihydroxycholecalciferol and 1α,25-dihydroxycholecalciferol at 10 nM concentration. Then the cells were fixed, stained with PI and read with flow cytometer. Treatment of cells with 20,23-dihydroxycholecalciferol resulted in similar changes in the distribution of cells in different cell cycle phases compared to 1α,25-dihydroxycholecalciferol. Data is presented as mean±SD (n=3), p<0.05 between control and treatment (FIG. 31).

### Involucrin Expression is Stimulated and Keratinocyte Size and Granularity Increase

20,23-dihydroxycholecalciferol stimulated expression of involucrin gene similar with the action of 1α,25-dihydroxycholecalciferol (calcitriol). To measure the expression of involucrin both flow cytometry and microscopy were utilized. Cells were treated for 24 h with 20,23-dihydroxycholecalciferol and 1α,25-dihydroxycholecalciferol at 10 nM concentration. As shown in FIGS. 32A-32B, treatment of HaCaT keratinocytes resulted in the increase of expression of involucrin compared to control. Moreover, the effects of 20,23-dihydroxycholecalciferol on forward and side scatter were measured. These parameters reflect cell size and granularity, respectively. Both parameters are known to increase during keratinocyte differentiation. As shown in Table 12, 20,23-dihydroxycholecalciferol increased significantly both forward and side scatter of HaCaT keratinocytes. 1α,25-dihydroxycholecalciferol acted similarly, but only the effect on forward scatter was statistically significant.

TABLE 12

| HaCaT | Forward scatter | Side scatter |
|---|---|---|
| 10⁻⁸ [M] | [mean signal intensity] | [mean signal intensity] |
| 0 | 14.43 ± 6.79 | 143.6 ± 21 |
| 20,23(OH)₂D3 | 87.74 ± 29.18* | 154.4 ± 30* |
| 1,25(OH)₂D3 | 122.12 ± 23.98** | 143.46 ± 15.76 |

### Expression of CYP24 in Keratinocytes is Poorly Stimulated, but Stimulates Expression of VDRE

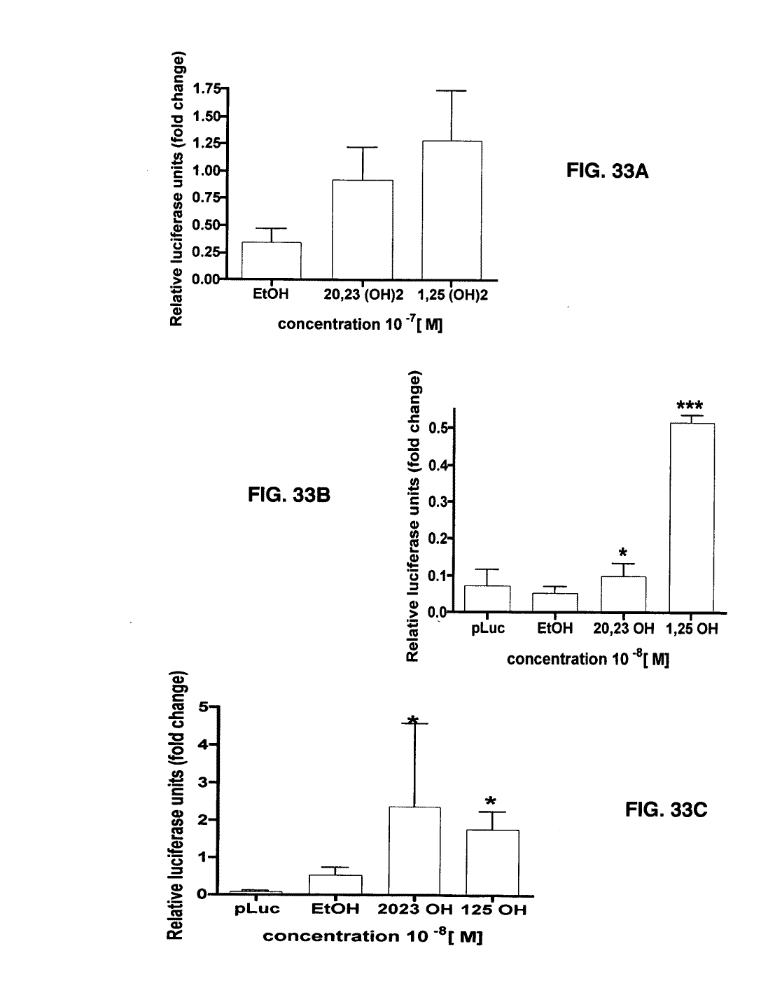

Since CYP24 is an important physiological target of 1α,25-dihydroxycholecalciferol in the kidney and peripheral tissues including skin the action of 20-hydroxycholecalciferol was compared with the action of calcitriol on the transcriptional activity of CYP24 promoter in HaCaT keratinocytes. HaCaT keratinocytes were transfected with either luciferase reporter construct driven by CYP24 promoter or promoterless luciferase construct pLuc, or vitamin D responsive element. As shown in FIGS. 33A-33C, 20,23-dihydroxycholecalciferol did not affect activity of promoterless (pLuc) construct. 1α,25-dihydroxycholecalciferol stimulated transcriptional activity of CYP24 promoter and 20,23-dihydroxycholecalciferol stimulated activity of CYP24 promoter poorly. But in excess of human vitamin D receptor this stimulation is increased. This documents that 20,23-dihydroxycholecalciferol has very low effect on 1α,25-dihydroxycholecalciferol metabolism in comparison to 1α,25-dihydroxycholecalciferol itself. Activation of VDRE indicates that mechanism of its action is in part mediated through vitamin D receptor.

### NFκB Binding Activity is Attenuated

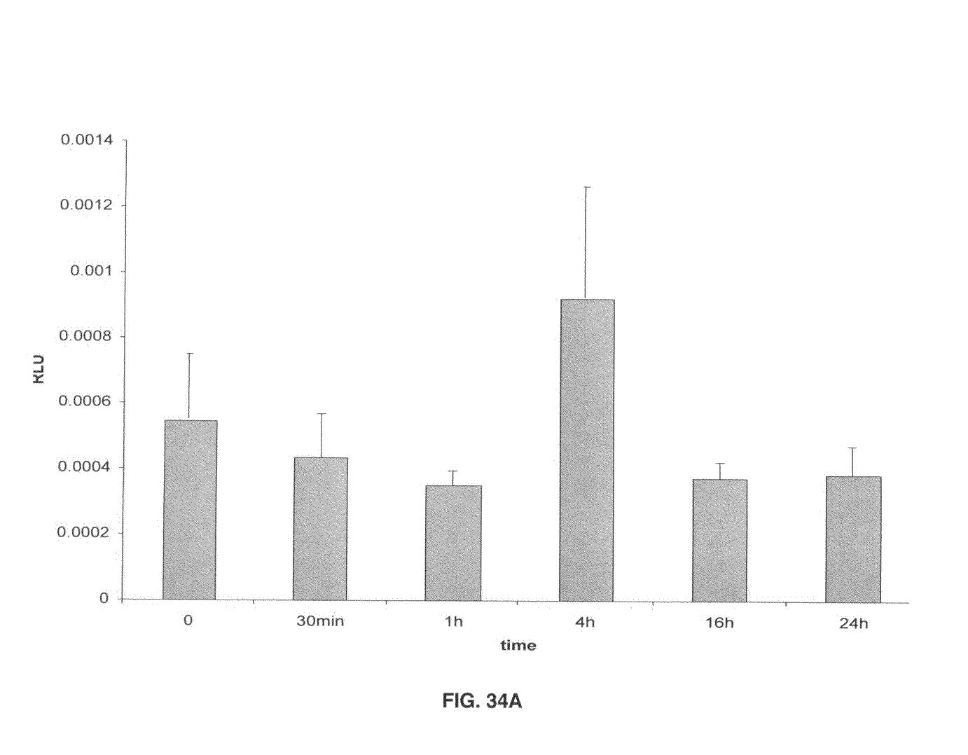

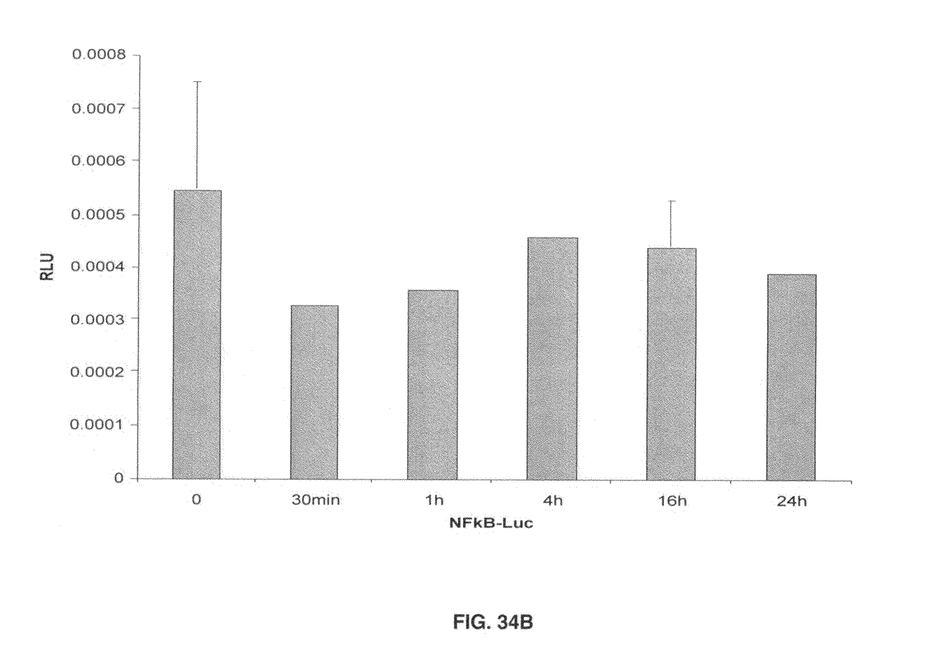

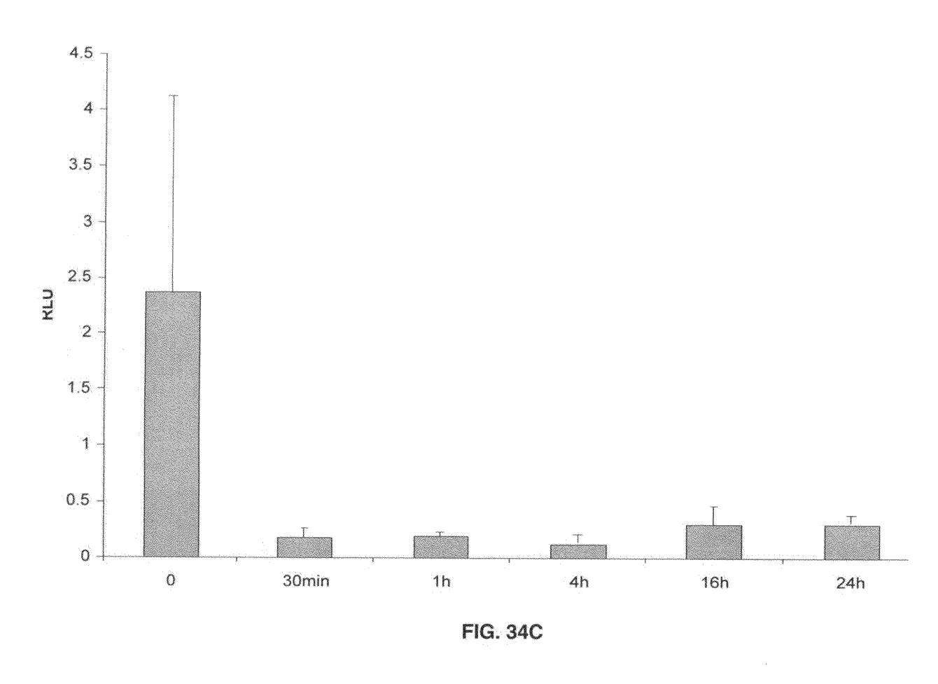

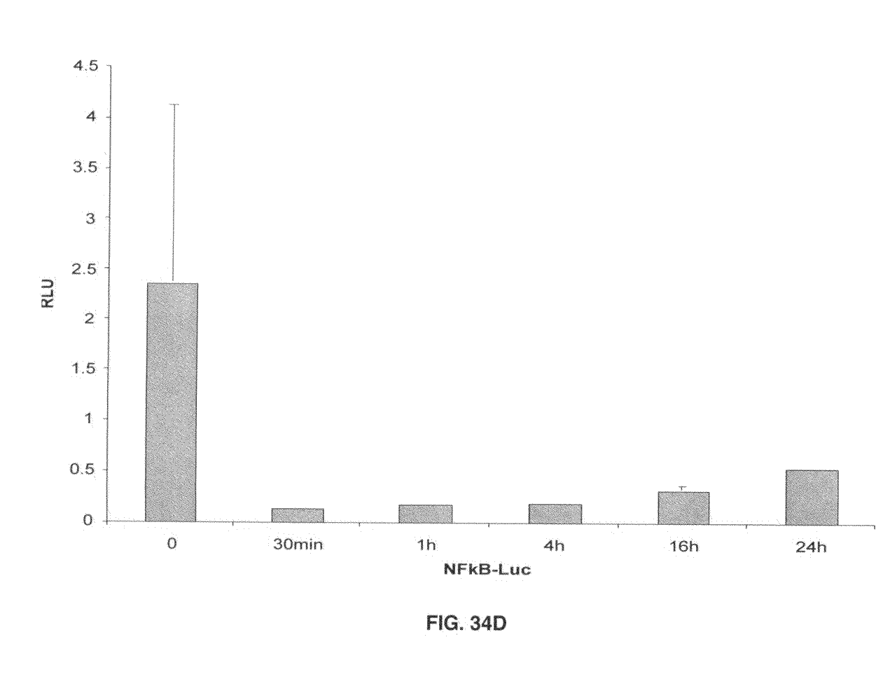

The DNA-binding activity of NFκB in HaCaT keratinocytes (FIG. 34A-34B) and normal human keratinocytes (FIG. 34C-34D) was measured by EMSA. Nuclear extracts from HaCaT cells treated with 20,23-dihydroxycholecalciferol were incubated with NFκB oligo and subjected to electrophoresis. 20,23-dihydroxycholecalciferol inhibited the NFκB binding activity in this assay. Confirmatory results showing inhibition by 20,23-dihydroxycholecalciferol were obtained using NFκB-Luc construct.

### NFκBI (IκBα) Protein Levels Increase in HaCaT and Normal Keratinocytes

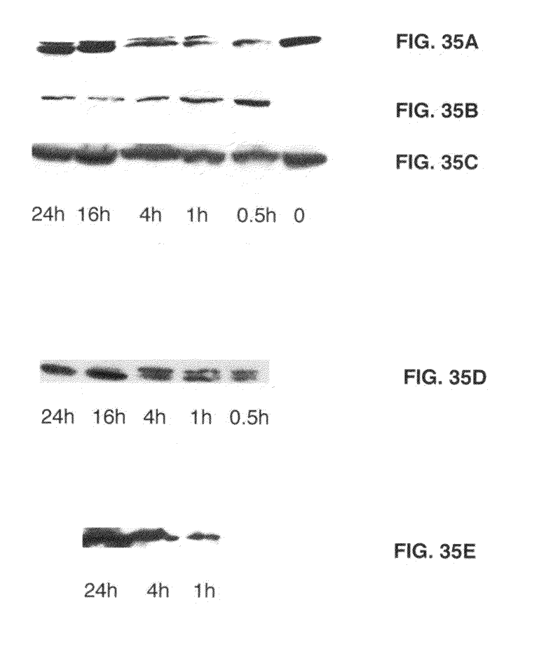

20,23-dihydroxycholecalciferol induced an increase in NFκBI (IκBα) protein levels in HaCaT and normal keratinocytes in a time dependant fashion, while the expression of NFκB activity remained unchanged. These actions are the same as with 1α,25-dihydroxycholecalciferol. The inhibitory effect of 20,23-dihydroxycholecalciferol on NFκB activity can be in part explained by stimulation of NFκB inhibitor (IκB) activity (FIGS. 35A-35E).

### EXAMPLE 8

### Effect of Various Compounds on Normal and Cancer Cells

### Hydroxy Derivatives of Cholecalciferol (Vitamin D3) on Keratinocyte Proliferation

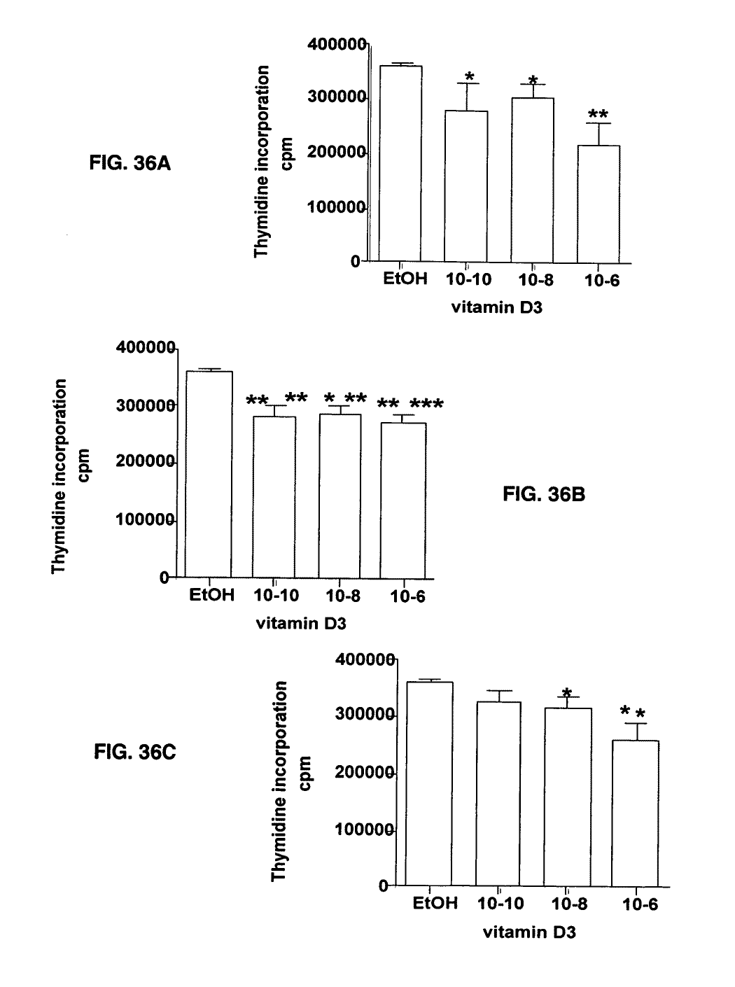

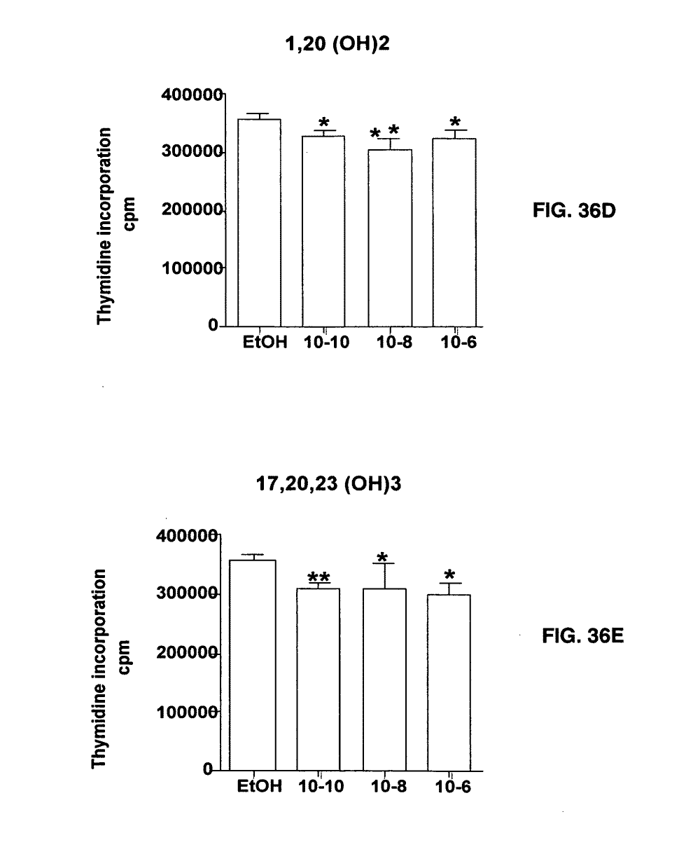

Inhibition of keratinocytes proliferation by 17α,20,23-trihydroxycholecalciferol (FIG. 35E) in comparison to 20-hydroxycholecalciferol (FIG. 36A), 20,23-dihydroxycholecalciferol (FIG. 36B), 1α,20-dihydroxycholecalciferol (FIG. 36D) and control compound 1α,25-dihydroxycholecalciferol (FIG. 36C) was examined. Normal human keratinocytes (HaCaT keratinocytes) were cultured in the presence of radioactive thymidine and after 48 hours DNA synthesis was measured as in Example 1. 17α,20,23-trihydroxycholecalciferol, 20-hydroxycholecalciferol, 20,23-dihydroxycholecalciferol and 1α,20-dihydroxycholecalciferol clearly inhibit keratinocyte proliferation more potently than the control compound 1α,25-dihydroxycholecalciferol.

### 20OH pD3 and 20OH pL3 Compounds

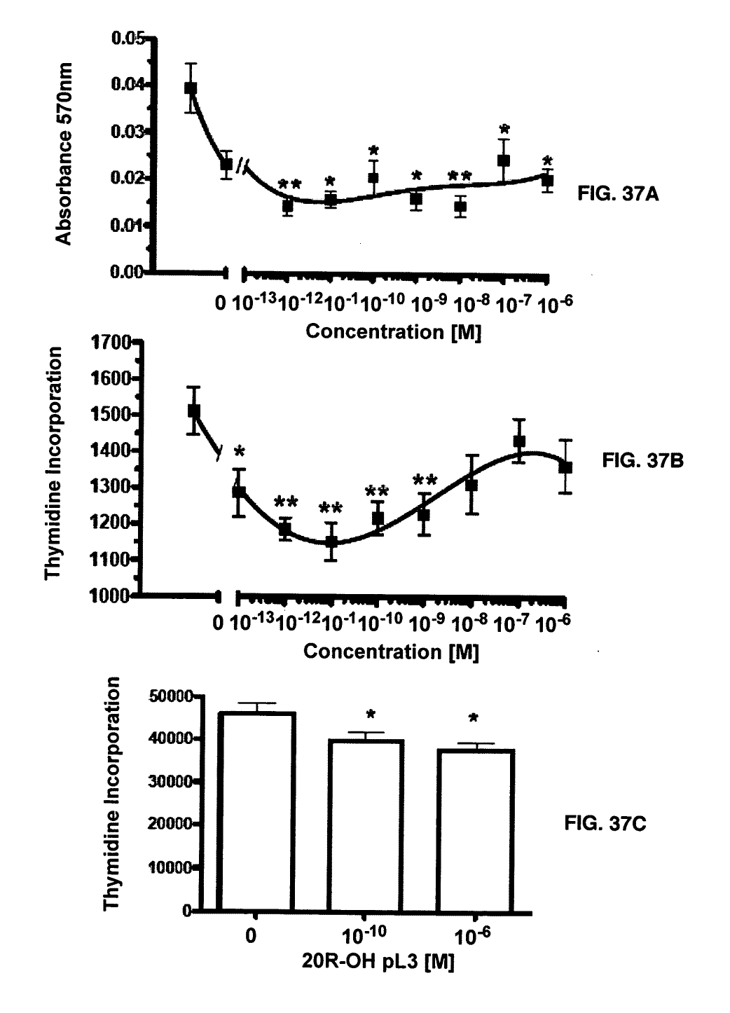

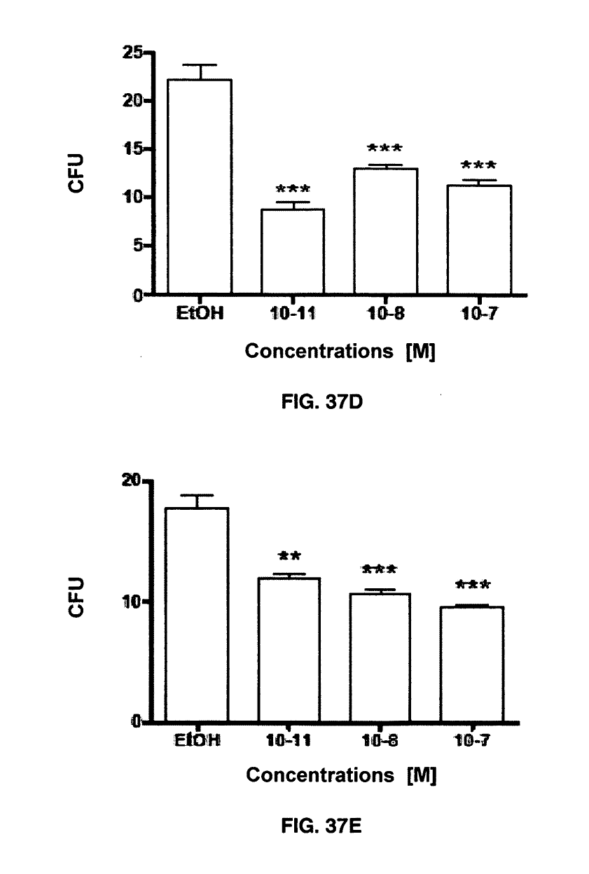

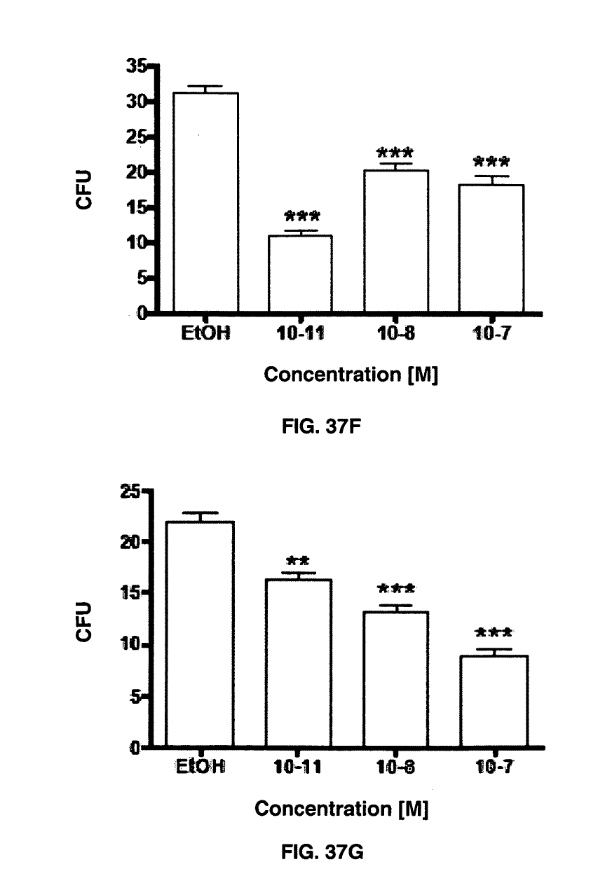

20OH pD3 and 20OH pL3 inhibit proliferation of SKMEL-188 human melanoma cells and epidermal HaCaT keratinocytes in a dose dependent manner as measured by MTT test after 48 hrs of culture and by DNA synthesis after 24 hrs (FIGS. 37A-37C), respectively. Also, 20OHpD3 and 20OHpL3 inhibit the growth on soft agar of AbC1 melanoma cells (FIGS. 37D-37G), respectively. A Colony Forming Unit Assay measuring colonies greater than 0.2 and 0.5 mm was performed after 3 weeks.

### pD3 and aD3 Compounds

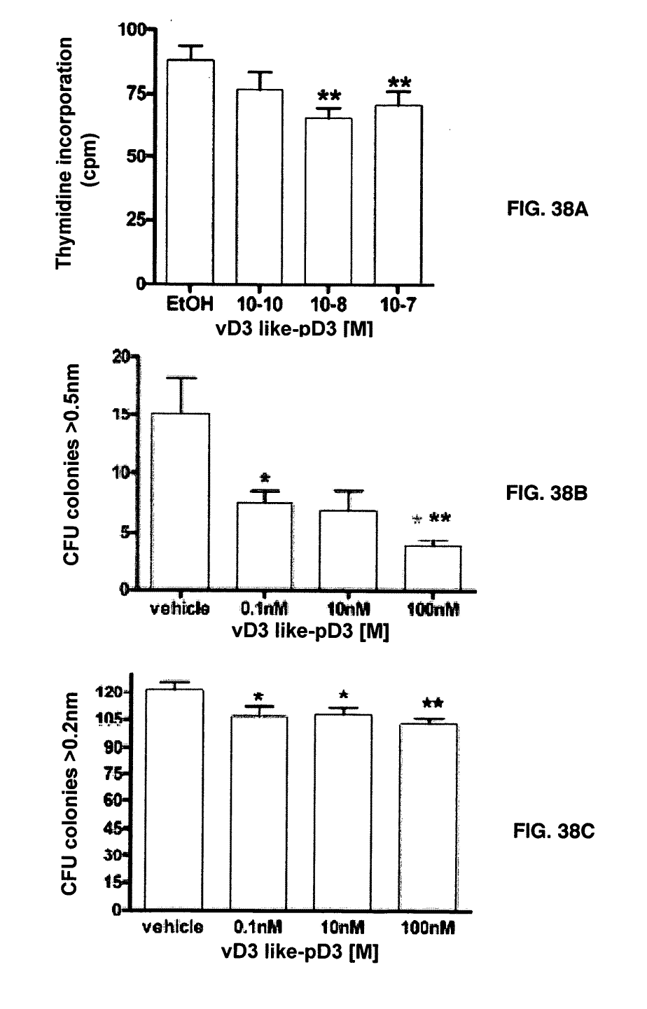

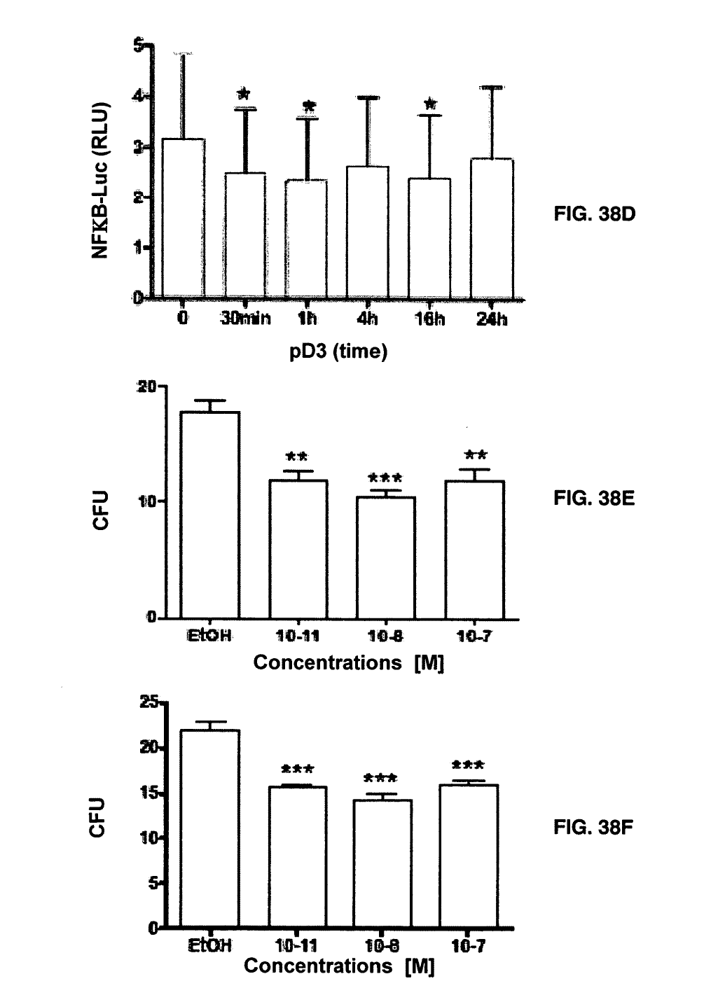

The vitamin D3-like compound pD3 inhibits proliferation, i.e., DNA synthesis of epidermal HaCaT keratinocytes after 48 hr of culture (FIG. 38A). Also, increasing concentrations of pD3 and aD3 suppress colony formation on soft agar, in a dose dependent manner, of SKMEL-1188 human melanoma cells (FIGS. 38B & 38D-38F), respectively, and PC3 human prostate cancer cells (FIG. 38C).

pD3 inhibits NFκB-Luc activity in HaCaT keratinocytes (FIG. 38D). HaCaT cells were transfected with luciferase construct NFκB-Luc. 24 h post-transfection cells were treated for the indicated period of time with ethanol as a vehicle and pD3 at 100 nM concentration. Then cells were lysed and luciferase activity was measured. Decreased activity of the construct indicates anti-inflammatory action, since NFκB is a positive regulator of immune activity.

### 17,20-diOHpL3 and 17,20-diOH pD3

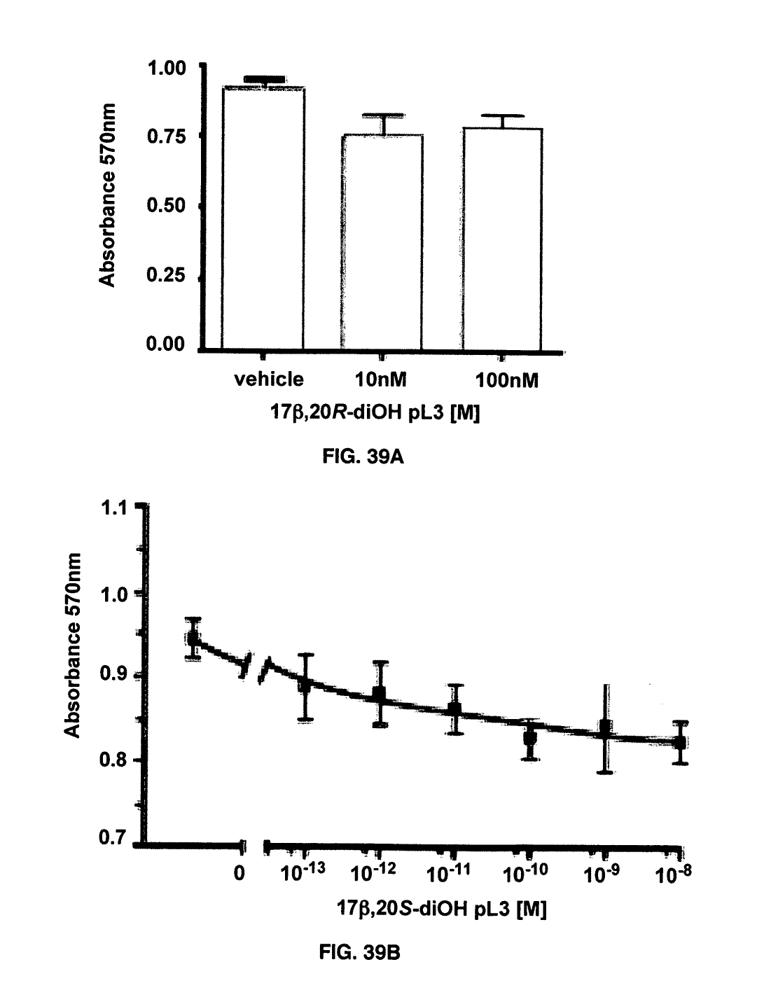

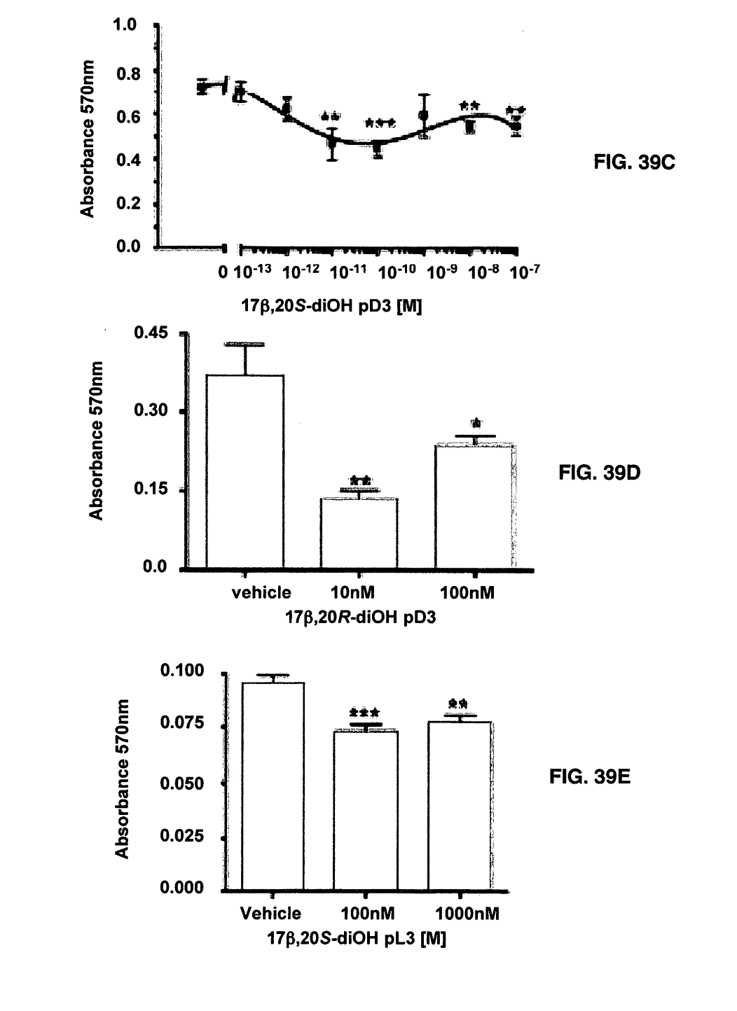

17,20-diOHpL3 and 17,20-diOHpD3 inhibit proliferation of epidermal HaCaT keratinocytes (FIGS. 39A-39E), respectively and melanoma cells (FIG. 39E). Cell proliferation was measured with an SRB assay after 48 hr of culture.

### 17-COOH Acid Inhibits DNA Synthesis

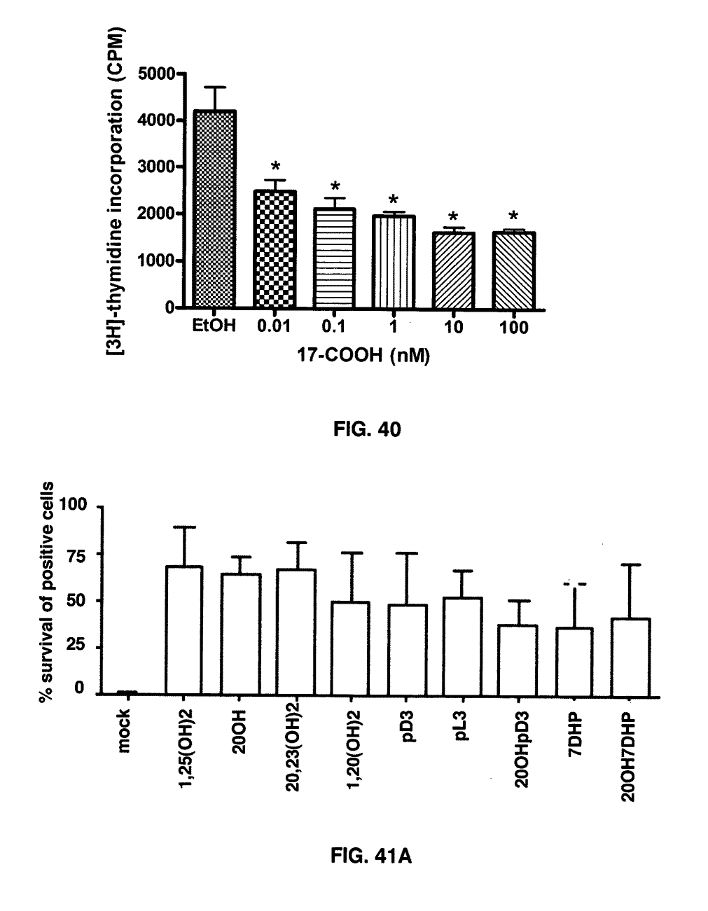

HaCaT keratinocytes were incubated for 72 hrs in DMEM medium containing 5% charcoal treated FBS and 17-COON at 0.01 nM, 0.1 nM, 1.0 nM, 10 nM, and 100 nM followed by [3H]-thymidine treatment for 4 hrs. DNA synthesis was inhibited at all concentrations compared to ethanol control (FIG. 40).

### Induction of Cell Differentiation

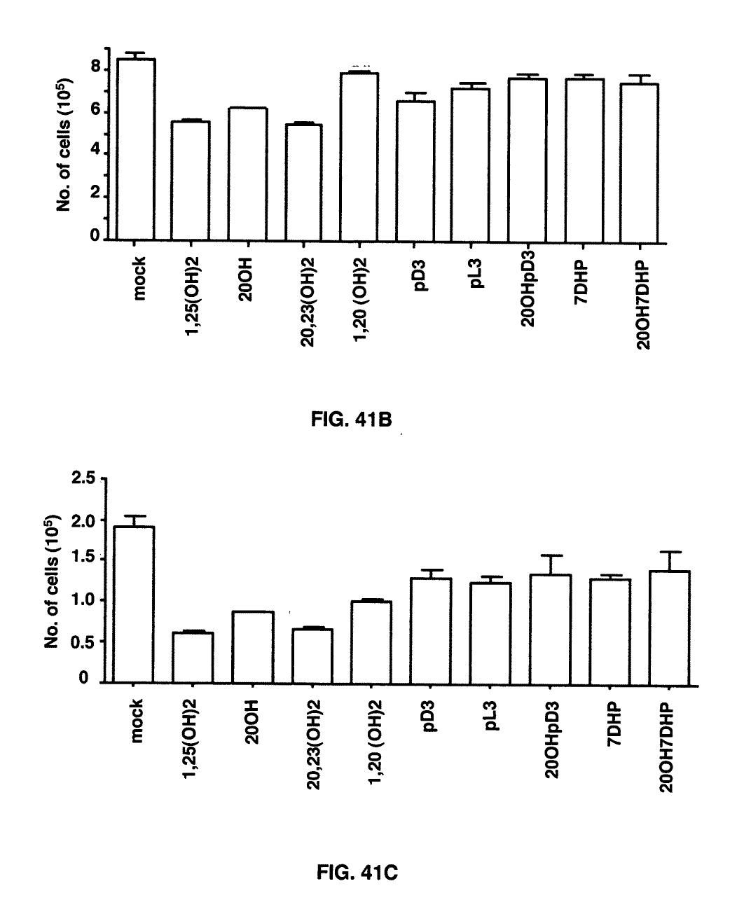

Human chronic myeloid leukemia cells (K562) and mouse erythroleukemia cells (Mel) were treated with compounds pD3, 20-HpL3 and 20-OHpD3 at 10-7 M concentration for 7 days and number of viable cells determined. pD3, 20-HpL3 and 20-OHpD3 induce differentiation of K562 human chronic myeloid leukemia cells (FIG. 41A) and inhibit proliferation of K562 cells (FIG. 41B) and mouse erytholeukemia cells (Mel) (FIG. 41C)

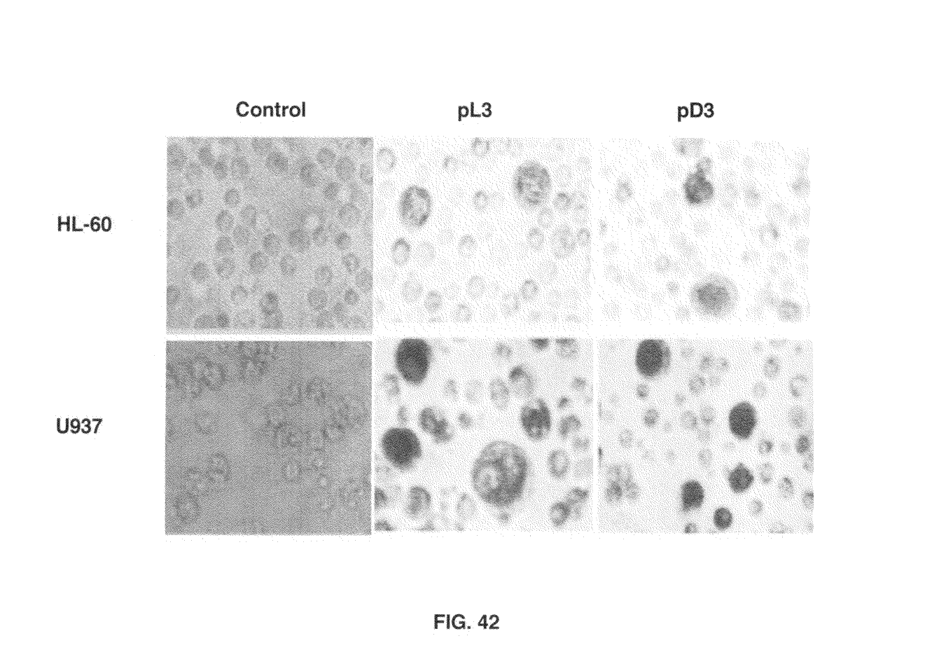

Also compounds pD3 and 20-HpL3 (FIG. 42) induce monocytic differention in HL-60 and U937 human leukemia cell lines as evidenced by the appearance of monocytic cells compared to control under the microscope. For monocytic determination an NBT staining was performed. NBT-positive cells (blue color) are visible after treatment compared to untreated control cells.

### EXAMPLE 9

### Effects of 20(OH)D2 on Arthritis and Scleroderma

### Type II Collagen-Induced Arthritis (CIA) Model of RA

The CIA model in DBA/1 Lac J mice has been widely studied as a model with some features of human RA, and has served as a reliable model to study various mediators and therapies of autoimmune arthritis. The immunization of DBA/1 Lac J mice with native bovine CII in complete Freund's adjuvant (CFA) is followed 10-14 days later by the onset of arthritis in the distal extremities. The arthritis, characterized by joint swelling and redness, is accompanied and largely induced by increases in serum antibodies to CII. The arthritis is characterized by acute, subacute and chronic inflammation that correlates with histologic changes in distal extremity joints that progressively worsens during the subsequent 10 to 40 day period. The arthritis is dependent on the generation of inflammatory mediators from activation of the complement cascade by anti-CH antibodies, the infiltration of neutrophils, monocytes and T cells into the joint resulting in liberation of inflammatory cytokines and various proteases (42).

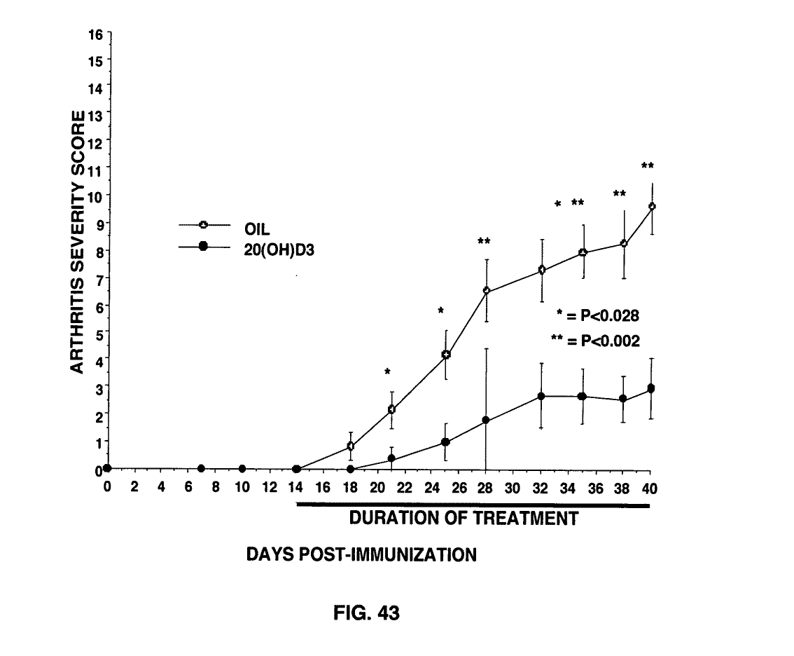

Twenty-four DBA/1 Lac J female mice 6 wks old were immunized with bovine CII in CFA. On day 14 post-immunization, 12 mice were given 50 μl sterile sesame oil i.p. (oil group 0) and 12 mice were given 50 μl sesame oil containing 50 ng 20(OH)D₃ every day till day 40 post-immunization. Arthritis severity was assessed every 3-4 days by two observers and each paw was given a score of 0=no swelling; 1=slight swelling and redness; 2=moderate swelling and redness; 3=marked swelling and redness; and 4=marked swelling and redness with deformity. Total maximum score per mouse being 16. FIG. 43 shows that 20(OH)D₃ given i.p. every day at 2.5 μg/kg dose beginning day 14 post CII immunization suppresses CIA in DBA/1 Lac J mice (FIG. 43).

### EXAMPLE 10

### Vitamin D Analogues 20(OH)D₃, 17,20(OH)₂D₃, 20,23(OH)₂D₃ Modulate Cytokine/Chemokine

### Production by Cultured Murine Spleen Cells

Vitamin D analogues were solublized in absolute alcohol (EtOH) and added at 1:100 dilution to cultures of spleen cells from normal DBA/1 Lac J mice (Table 13). There was down modulation of Th1 cytokines (IFNγ, GMCSF, IL-6 and Th17 and inflammatory cytokines G-CSF and IL-1α. Chemokines MCP-1, KC, and IP-10 were all down regulated to varying degrees. Th2 cytokine (IL-10) production was increased by 20(OH)D₃ and 20,23(OH)₂D₃. These data provide strong in vitro evidence to indicate immunomodulatory effects of these three selected novel vitamin D analogues will have in the CIA model. Viability was assessed by trypan blue exclusion of the cultured splenocytes at the time of harvest of the supernatants. The % viable cells were the same in wells using EtOH vehicle as in those with vitamin D analogues solubilized in the same volume of EtOH. Therefore, changes are not due to decreased cell viability. In additional studies using normal human peripheral blood mononuclear cell (PBMC) cultures, it was found that 20(OH)D₃ markedly reduced TNFα production induced by LPS (10 μg/ml) [vehicle=6002±1479 pg/ml; 20(OH)D3 10⁻⁸M=2609±1961 pg/ml p<0.01].

Table 13 shows the activity of vitamin D analogs to modulate cytokines and chemokines in anti-CD3 stimulated DBA/1 Lac J spleen cells in vitro. Spleen cells from three normal 8 wk old female DBA/1 Lac J mice were cultured in 96 well tissue culture plates in quadruplicate at 2×10⁶ cells/ml in RPMI 1640 medium containing 9% FCS with and without anti-CD3 (4 pg/ml) and with anti-CD3+10⁻⁷M natural vitamin D analogues for 5 days after which time supernatants were harvested and subjected to cytokine multiplex on a Luminex instrument using Milliplex Mouse Kit (values are pg/ml). The general trend in changes in cytokine levels were similar for each of the mice but some produced different levels of each cytokine for each culture additions. Data from one mouse are given and bold numbers indicate those that changed from anti-CD3 stimulated culture with vitamin D analogues added. There was no significant stimulation of IL-1g, IL-2, IL-4, IL-5, IL-7, IL-9, IL-12 p70, IL-13, IL-15, MIP-2, RANTES or TNFα with the anti-CD3 MOAB.

TABLE 13

| Spleen Cells + | | GM- | | | | | | | | |
|---|---|---|---|---|---|---|---|---|---|---|
| Additions | IFNγ | CSF | IL-6 | IL-17 | IL-10 | MCP-1 | KC | G-CSF | IP-10 | IL-1α |
| ETOH + PBS | 14 | 44 | 25 | 30 | 1 | 138 | 8 | 14 | 4 | 2 |
| ETOH + Anti-CD3 | 709 | 109 | 174 | 78 | 24 | 357 | 153 | 276 | 184 | 24 |
| 1,25(OH)₂D₃ 10⁻⁷M + | 0 | 7 | 12 | 1 | 1 | 197 | 3 | 11 | 5 | 1 |
| Anti-CD3 | | | | | | | | | | |
| 20(OH)D₃ 10⁻⁷M + | 494 | 51 | 55 | 59 | 37 | 100 | 39 | 10 | 83 | 8 |
| Anti-CD3 | | | | | | | | | | |
| 20,23(OH)₂D₃ 10⁻⁷M + | 271 | 47 | 40 | 57 | 56 | 182 | 47 | 103 | 96 | 11 |
| Anti-CD3 | | | | | | | | | | |
| 17,20S(OH)₂pD₃10⁻⁷M + | 0 | 41 | 13 | 111 | 16 | 102 | 5 | 12 | 6 | 4 |
| Anti-CD3 | | | | | | | | | | |

### EXAMPLE 11

### Secosteroid Inhibition of TGF-b-Induced Collagen and Hyaluronan Production by Fibroblasts

Human dermal fibroblasts grown from explant skin cultures at less than 10 subpassages were plated at 5×10⁴ cells per well in 24 well Costar tissue culture plates and were grown to confluency. Complete MEM was then changed to serum free Complete MEM without non-essential amino acids. After 24 hours, culture medium was changed (450 pl/well) to the same and secosteroids listed in Table 13 were added in 10 pl absolute alcohol (ETOH) to a final concentration of 10⁻⁹ and M, 3 replicate wells each. Vehicle control wells (n=6) contained 10 μl (ETOH). After 2 hour pre-incubation, hr TGF-31 (R and D systems) was added to each well except ETOH wells at a final concentration of 5 ng/ml. After 48 hours of culture, plate wells were paused with 1 μCI ³[H]-proline. After 24 hours, culture supernatants were harvested and collagenase sensitive protein was determined as previously described (44). Results in Table 14 shows that pD₃, 17,20(OH)₂7DHP, 17,20S(OH)₂pD₃, 17,20S(OH)₂pL₃, 20(OH)D₃ and 1,25(OH)₂D₃ inhibited TGF-b1 induced collagen protein production.

In a similar separate study using a different human dermal fibroblast line, it was that observed these same secosteroids and 1,25(OH)₂D₃ inhibited TGF-β1 induced hyaluronan synthesis at a concentration of 10⁻¹⁰ M (Table 13). Similar inhibition was observed at 10⁻⁹M of each secosteroid and 1,25(OH)₂D₃ but only data at 10⁻¹⁰ M are shown. There were found no significant differences in fibroblast numbers per well and no significant differences in trypan blue exclusion between control wells vs those with secosteroids range 95-100% (data not shown).

TABLE 14

| | Collagen CPM/Well | | Hyaluronan CPM/Well | |
|---|---|---|---|---|
| Condition | (mean ± SEM) × 10⁻⁵ | | (mean ± SEM) × 10⁻⁵ | |
| hrTGF-b1 5 ng/ml secosteroids at 10⁻¹⁰ M | FB | p Value | FB | p Value |
| PBS + ETOH | 248 ± 21 | | 646 ± 77 | |
| TGF-β1 + ETOH | 829 ± 17* | <0.001 | 1556 ± 51* | <0.001 |
| TGF-β1 + 7DHP | 292 ± 31^{†} | <0.001 | 230 ± 34^{†} | <0.001 |
| TGF-β1 + pD3 | 181 ± 14^{†} | <0.001 | 135 ± 25^{†} | <0.001 |
| TGF-β1 + 17,20 R(OH)₂7DHP | 173 ± 11^{†} | <0.001 | 607 ± 127^{†} | <0.001 |
| TGF-β1 + 17,20 R(OH)₂pD3 | 330 ± 17^{†} | <0.001 | 163 ± 8^{†} | <0.001 |
| TGF-β1 + 17,20 R(OH)₂ pL3 | 266 ± 6^{†} | <0.001 | 97 ± 46^{†} | <0.001 |
| TGF-β1 + 17,20 S(OH)₂7DHP | 213 ± 17^{†} | <0.001 | 227 ± 53^{†} | <0.001 |
| TGF-β1 + 17,20 S(OH)₂pD3 | 95 ± 10^{†} | <0.001 | 662 ± 189^{†} | 0.01 |
| TGF-β1 + 17,20 R(OH)₂ pL3 | 320 ± 3^{†} | <0.001 | 222 ± 13^{†} | <0.001 |
| TGF-β1 + 20 (OH) D3 | 222 ± 47^{†} | <0.001 | 234 ± 17^{†} | <0.001 |
| TGF-β1 + 1,25 (OH)2 D3 | 310 ± 100^{†} | 0.007 | 297 ± 18^{†} | <0.001 |

^{†}Significantly
*Significantly different (p < 0.001) from PBS + ETOH different (p < 0.001) from TGF-b1 + ETOH

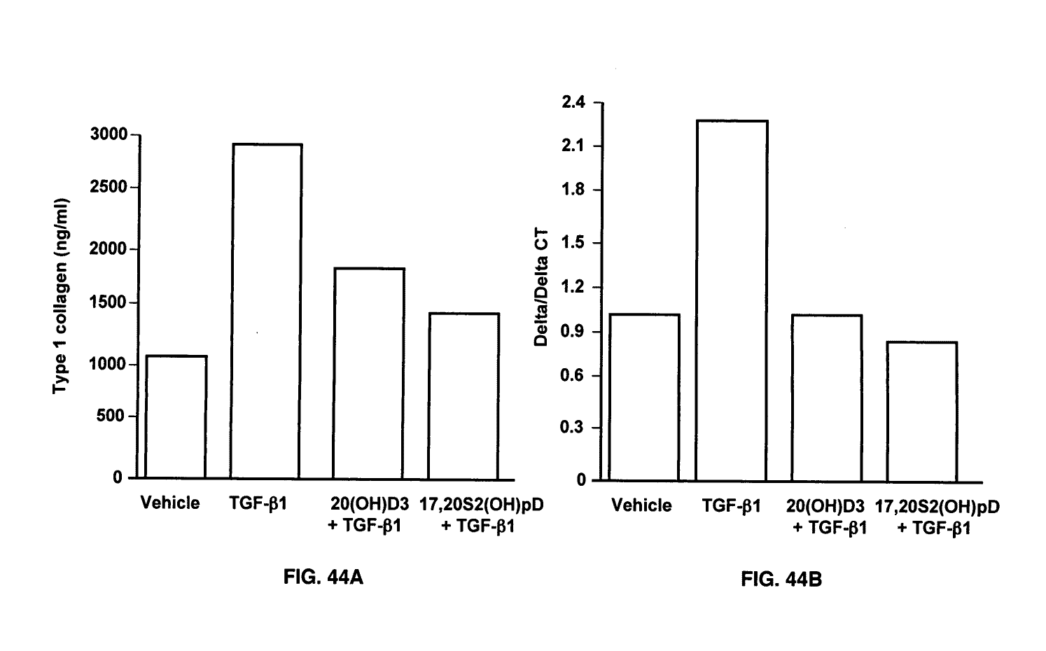

Additional studies using human skin fibroblasts were performed employing a type I collagen specific ELISA Chondrex, and real time RT PCR to quantitate type I collagen protein and Col1A1 mRNA expression in culture of human fibroblast stimulated by TGF-31 in the presence and absence of 17,20S(OH)₂pD₃ and/or 20(OH)D₃. These studies confirmed that Type I collagen protein production that was induced by TGF-β1 and that Col1A1 mRNA that was induced by TGF-β1 were suppressed by these analogues (FIGS. 44A-44B).

### EXAMPLE 12

### 20(OH)D₃ Prevents Bleomycin-Induced Scleroderma in Mice

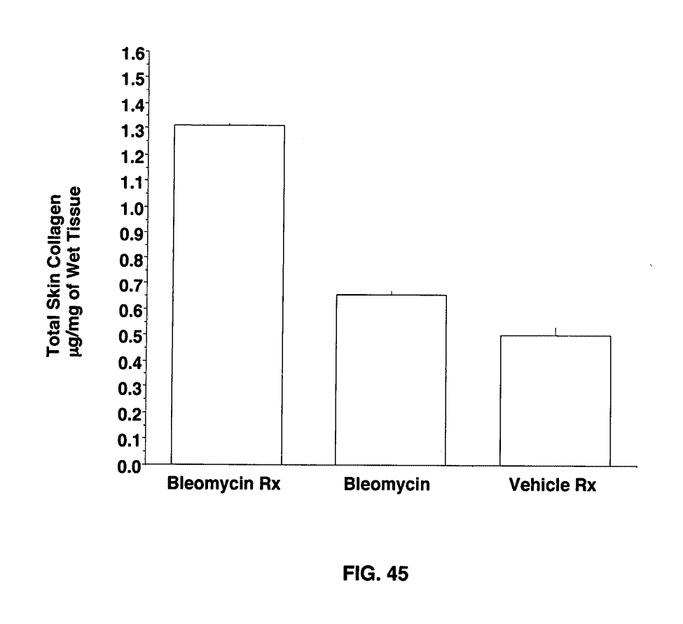

Groups of mice (5 each) were assigned to receive either: Vehicle (50 μl sesame oil i.p.100μl saline S.C.); bleomycin (180 μg/100 μl bleomycin+50 μl sesame oil i.p.); or bleomycin+20(OH)D₃ (180 μg/100 μl bleomycin+50 μg 20(OH)D₃/50 μl sesame oil i.p) daily for 21 days. The skin was injected S.C. with 20(OH)D₃ or vehicle daily within the same 1.5 cm² area. On day 22, all mice were euthanized and skin in the shaved area of the back was treated with a depilatory agent after which a biopsy of 1 cm circumference encompassing the S.C. injection site was taken to a depth to include the full thickness of the dermis. The skin samples from 5 randomly selected mice from each group were weighed and snap frozen in liquid nitrogen. Later, the skin samples were thawed and treated overnight with pepsin (0.1 mg/ml) of 0.5 M acetic acid at 4° C. with constant rocking to remove terminal non-helical telopeptides to release the collagen into solution. Total solubilized collagen was quantitative using a Sircol Collagen Assay kit using type I bovine collagen to obtain a standard curve. The collagen content of the skin samples was expressed as pg of collagen per mg tissue weight. Results for the groups were expressed as mean±SEM and analyzed by one way ANOVA with p values<0.05 considered to be statistically significant. As shown in FIG. 45 total collagen significantly increased more than 2 fold with bleomycin treatment and this was prevented with 20(OH)D₃ treatment at 3 μg/kg daily dose. The bleomycin treated mice weighed 21% less than the vehicle control group whereas the bleomycin treated mice that were treated with 20(OH)D₃ weighed 13.6% less than the vehicle control group.

### EXAMPLE 13

### Effect of Various Vitamin D2 Derivative or Analog Compounds on Normal and Cancer Cells

### 20(OH)D₂ Induces Differentiation of Normal Human Epidermal Keratinocytes

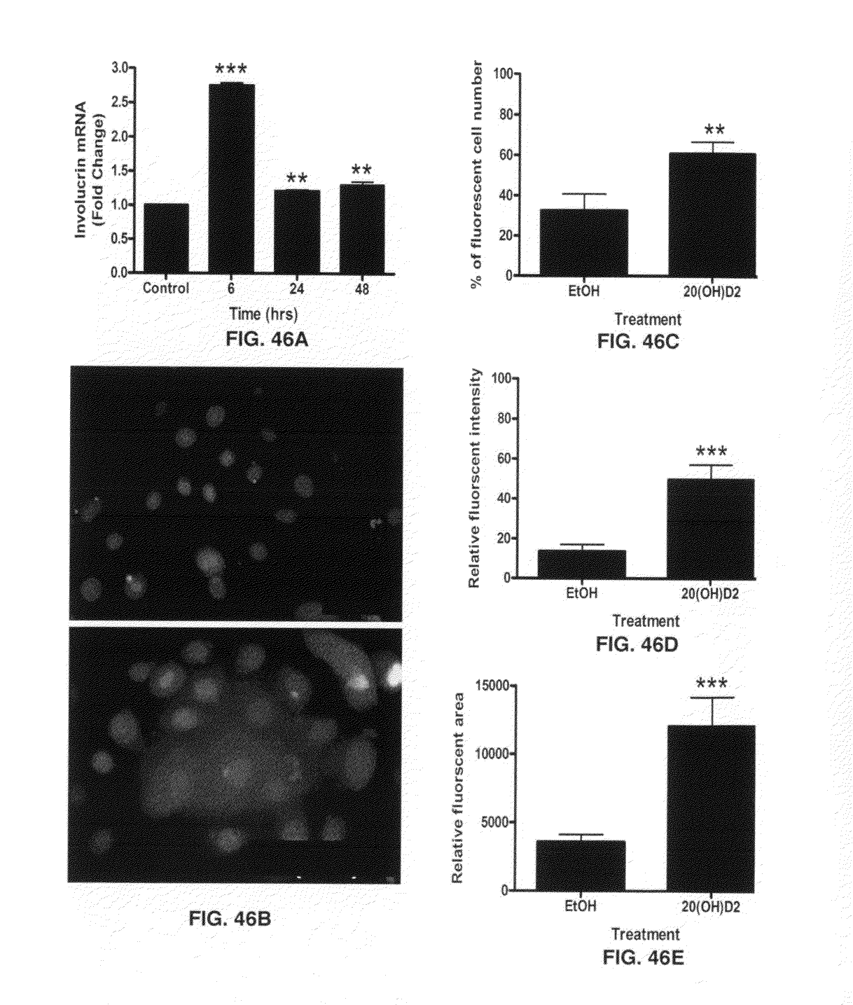

20(OH)D₂ (10⁻⁷ M) induced time dependant involucrin gene expression (FIG. 46A) that was accompanied by increased expression of involucrin protein (FIG. 46B; the top shows fluorescence in the presence of ethanol vehicle and the bottom shows fluorescence in the presence of 20(OH)D₂), which was significant in terms of increased number of cells expressing involucrin (FIG. 46C), increase in total relative fluorescence (FIG. 46D) and increased fluorescent area (FIG. 46E). Data are shown as mean±SD (n=10-20). **, p<0.01. ***, p<0.001.

### 20(OH)D₂ Inhibits Melanoma Cell Colony Formation Better than 1.25(OH)₂D₃

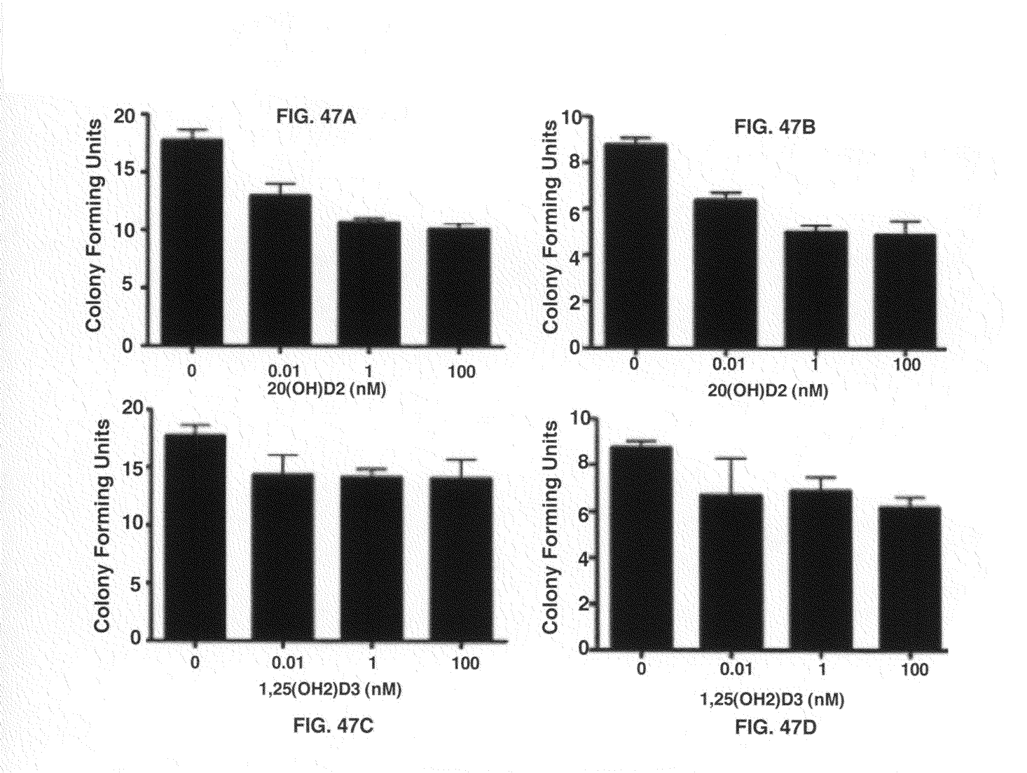

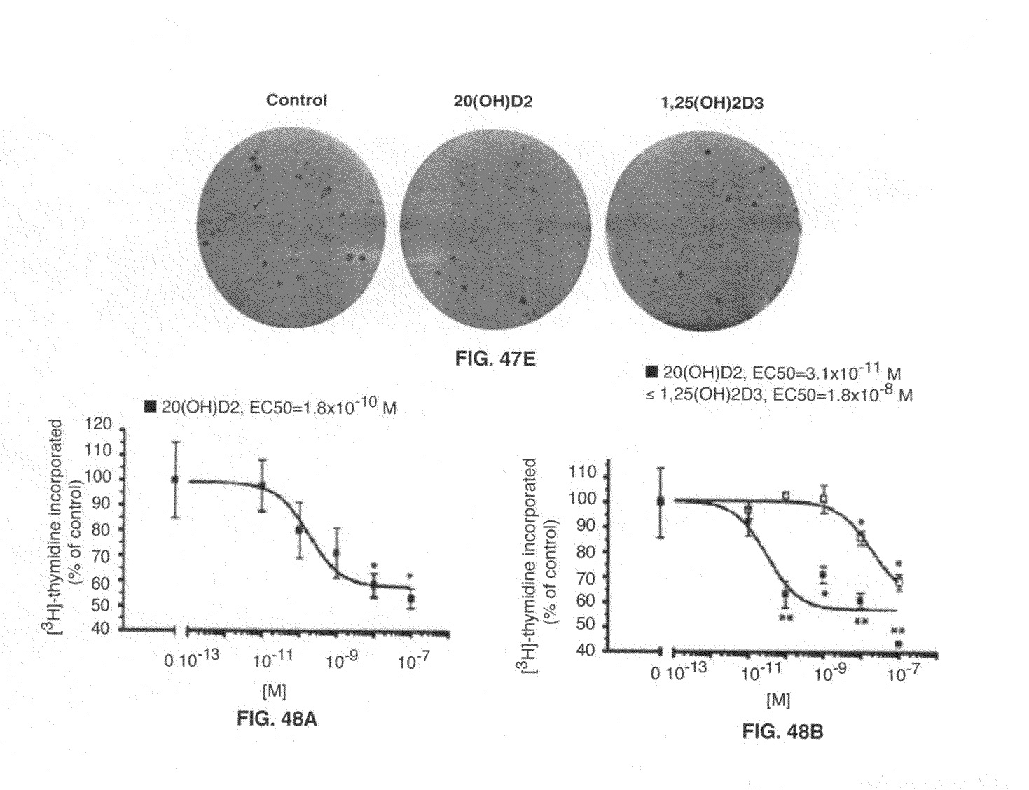

Inhibitory effects of 20(OH)D₂ (FIGS. 47A-47B) in comparison to 1,25(OH)₂D₃ (FIGS. 47C-47D) on the ability of human melanoma cells to form colonies was examined. After 7 days, colonies were stained with crystal violet and the numbers over 0.2 mm (FIGS. 47A, 47C) and over 0.5 mm (FIGS. 47B, 47D were counted. Data are shown as mean±SD (n≧3). *, p<0.05; **, p<0.01. ***, p<0.001. FIG. 47E shows representative plates of melanoma cells treated with vehicle (control) or 10⁻⁷ M 20(OH)D₂ or 1,25(OH)₂D₃.

### 20(OH)D₂ Derivatives Inhibit DNA Synthesis in Normal and Malignant Skin Cells

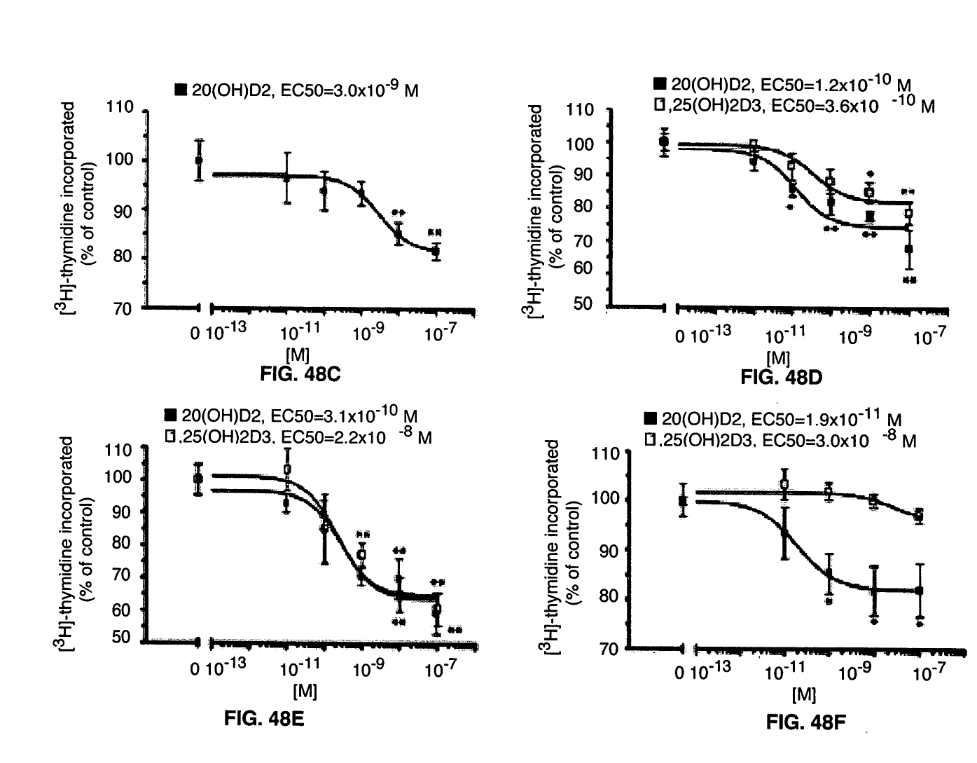

HaCaT keratinocytes were an incubated for 48 h (FIG. 48A) or 72 h (FIG. 48B) in the presence of graded concentrations of 20(OH)D₂ and/or 1,25(OH)₂D₃. Dose dependent inhibition of the proliferation of immortalized normal epidermal melanocytes (PIG1 line) (FIG. 48C), neonatal epidermal melanocytes (FIG. 48D), SKMEL-188 human (FIG. 48E) and AbC1 hamster (FIG. 48F) melanoma cells by 20(OH)D₂ or 1,25(OH)₂D₃ was measured after 72 h of exposure, with except for FIG. 48D, where it was measured after 48 hours. Data are shown as mean±SD (n 3); *, p<0.05; **, p<0.01.

The following references are provided herein:
1. Holick, M F and Clark, M B, (1978) Federation proceedings, 37:2567-2574.
2. Holick, M F, (2003) Journal of Cellular Biochemistry, 88:296-307.
3. Holick et al. (1995) PNAS (USA), 92:3124-3126.
4. Holick, M F, (2000) Clinics in Laboratory Medicine, 20:569-590.
5. Holick, M F, (2004) The American Journal of Clinical Nutrition, 80:1678S-1688S.
6. Guryev et al., (2003) Proc Natl Acad Sci USA, 100:14754-14759.
7. Slominski et al. (2006) Febs J, 273:2891-2901.
8. Slominski et al. (2005) Chem Biol, 12:931-939.
9. Slominski et al. (2005) Febs J, 272:4080-4090.
10. Slominski et al. (2004) Eur J Biochem, 271:4178-4188.
11. Feldman et al. Vitamin D, 2 ed. Elsevier Academic Press: Oxford, UK, 2005, 1952 pp.
12. Christakos et al. (2003) J Cell Biochem, 88:695-705.
13. Bikle (2006) www.endotext.com
14. Bikle (1986) J Clin Invest, 78:557-566.
15. Holick M F (2006) J Clin Invest, 116:2062-2072.
16. Lehmann B. (2005) Photochem Photobiol, 81:1246-1251.
17. Ebert (2006) Mol Cell Endocrino,I 248:149-159.
18. Ohyama Y. & Yamasaki T. (2004) Front Biosci, 9:3007-3018.
19. Bikle et al. (2004) J Steroid Biochem Mol Biol, 89-90:355-360.
20. Holick M F (2003) Recent Results Cancer Res, 164:3-28.
21. Wiseman H. (1993) FEBS Lett, 326:285-288.
22. Bikle et al. (2001) Mol Cell Endocrinol, 177:161-171.
23. Holick M F (2000) Clin Lab Med, 20:569-590.
24. Plum et al., PNAS (USA), 2004, 101:6900-6904.
25. Murari et al., Journal of Steroid Biochemistry, 1982, 17:615-619.
26. Slominski et al. (2003) J Cell Physiol, 196:144-153.
27. Vaisanen (2005) J Mol Biol, 350:65-77.
28. Pisarchik et al., (2004) Eur J Biochem, 271:2821-2830.
29. Slominski et al. (2004) Eur J Biochem, 271:4178-4188.
30. Zbytek et al. (2006) Mol Endocrinol, 20:2539-2547.
31. Marwah et al., Tetrahedron, 2003, 59:2273-2287.
32. Guo et al. (2003) Steroids, 68:31-42.
33. Siddiqui, Chemistry and Physics of Lipids, 1992, 63:115-129.
34. Webb et al. (1989) The Journal of Clinical Endocrinology and Metabolism, 68:882-887.
35. Fischer et al., Faseb J, 2006, 20:1564-6.
37. Bikle et al., J Steroid Biochem Mol Biol, 2005, 97:83-91.
38. MacLaughlin et al. (1982) Science, 216:1001-3.
39. Tuckey, R C & Stevenson, P M, (1984) Int. J. Biochem. 16:489-495.
40. Tuckey, R C & Stevenson, P M, (1984) Int. J. Biochem. 16:497-503.
41. Woods et al. (1998) Arch Biochem Biophys, 353:109-115.
42. Rosloniec et al. Collagen-induced arthritis. In: Current Protocols in Immunology, edited by Coico R, and Shevach E. New York: Wiley & Sons, 1997.
43. Raghow et al. (1987) J Clin Invest 79:1285-1288.

Any patents or publications mentioned in this specification are indicative of the level of those skilled in the art to which the invention pertains. Further, these patents and publications are incorporated by reference herein to the same extent as if each individual publication was specifically and individually incorporated by reference.

One skilled in the art would appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A steroidal compound that is an androsta-5,7-diene or a pregna-5,7-diene or an ultraviolet B (UVB) conversion product thereof or pharmaceutical compositions thereof.

2. The steroidal compound of claim 1, wherein the compound is identified in Table 1.

3. The steroidal compound of claim 2, wherein the compound of Table 1 is derivatized further to comprise an ether or an ester substituent.

4. The steroidal compound of claim 1, wherein the UVB conversion product is produced in vivo or in vitro.

5. A method for inhibiting proliferation of a cell, comprising:
contacting the cell with one or more steroidal compounds identified in one or both of Tables 1 or 2.

6. The method of claim 5, wherein the one or more steroidal compounds of Table 1 or Table 2 are derivatized further to comprise an ether or an ester substituent or are one or more of an androsta-5,7-diene or a pregna-5,7-diene, said compound converted in vivo to a corresponding ultraviolet B conversion compound after contacting the cell.

7. The method of claim 5, wherein the cell is a normally proliferating or abnormally proliferating adrenal cell, gonadal cell, keratinocyte or melanocyte, pancreatic cell, cell from the gastrointestinal tract, prostate cell, breast cell, lung cell, immune cell, hematologic cell, kidney cell, brain cell, cell of neural crest origin, skin cell, mesenchymal cell, leukemia cell, melanoma cell, or osteosarcoma cells.

8. The method of claim 5, wherein the cell is in vivo and is associated with a pathophysiological condition in a subject.

9. The method of claim 10, wherein the condition is associated with neoplastic cells.

10. The method of claim 11, wherein the condition is melanoma, squamous cell carcinoma, breast carcinoma, prostate carcinoma, lung carcinoma, sarcoma, carcinoma, lymphoma, leukemia, or brain tumor.

11. The method of claim 5, wherein the condition is a skin or mucosal disorder or a defect in cell differentiation.

12. The method of claim 11, wherein the skin disorder is a hyperproliferative skin disorder, a pigmentary skin disorder, an inflammatory skin disorder, or other skin disorder characterized by hair growth on legs, arms, torso, or face, or alopecia, or skin aging, skin damage or a pre-carcinogenic state.

13. The method of claim 12, wherein the hyperprofliferative skin disorder is psoriasis or a keloid or fibromatosis, the pigmentary skin disorder is vitiligo, the inflammatory or autoimmune skin disorder is pemphigus, bullous pemphigoid, allergic contact dermatitis, atopic dermatitis, or lupus erythematosus.

14. The method of claim 8, wherein the condition is associated with undifferentiated cells or defectively differentiated cells, said contact further inducing differentiation thereof.

15. The method of claim 14, wherein the condition results from an activity of NFkB directed against proliferating cells or immune cells.

16. The method of claim 15, wherein the condition is an autoimmune disease or an inflammatory process associated with NFκβ activity in keratinocytes, immunocompetent cells of the skin, the immune cells of the systemic immune system, or present in autoimmune diseases.

17. The method of claim 16, wherein the autoimmune disease or inflammatory process is scleroderma or morphea, keloid or fibromatosis, rheumatoid arthritis, multiple sclerosis, inflammatory bowel diseases, interstitial cystitis, diabetes, obesity atherosclerosis, vasculities, or gout.

18. The method of claim 8, wherein the condition is cosmetic, prophylaxis, or maintenance of healthy proliferating cells.

19. A method for producing one or more hydroxylated metabolites of (5Z,7E)-9,10-secocholesta-5,7,10(19)-trien-3β-ol (cholecalciferol) or (5Z,7E,22E)-9,10-secoergosta-5,7,9(10),22-tetraene-3β-ol (ergocalciferol), comprising:
hydroxylating a substrate of one or both of a cytochrome P450scc (CYP11A1) or CYP27B1 enzyme system in at least one position, said substrate enzymatically convertible to the hydroxylated cholecalciferol metabolite, said hydroxylase comprising a plant or animal hydroxylase having an activity that hydroxylates position C20 of a secosteroid or its 5,7-dieneal precursor.

20. The method of claim 19, wherein the substrate is cholecalciferol, ergocalciferol, (5Z,7E)-9,10-secopregna-5,7,10(19)-triene-1α,3β-diol or (5Z,7E)-9,10-secopregna-5,7,10(19)-triene-3β,20-diol.

21. The method of claim 19, wherein the hydroxylated cholecalciferol is (5Z,7E)-9,10-secocholesta-5,7,10(19)-triene-3β,20-diol, 9,10-secocholesta-5,7,10(19)-triene-1α,3β,20-triol, (5Z,7E)-9,10-secocholesta-5,7,10(19)-triene-3β,20,23-triol, 9,10-secocholesta-5,7,10(19)-triene-1α,3β,20,23-tetrol, (5Z,7E)-9,10-secocholesta-5,7,10(19)-triene-3β,17α,20,23-tetrol, or 9,10-secocholesta-5,7,10(19)-triene-1α,3β,17α,20,23-pentol.

22. The method of claim 19, wherein the hydroxylated ergocalciferol is (6E)-9,10-secocholesta-5(10),6,8-triene-3β,20α-diol, (6E)-9,10-secocholesta-5(10),6,8-triene-3β,20β-diol, 9β,10α-cholesta-5,7-diene-3β,20α-diol, 9β,10α-cholesta-5,7-diene-3β,20β-diol, cholesta-5,7-diene-3β,20α-diol, cholesta-5,7-diene-β,20β-diol, (5Z,7E,22E)-9,10-secoergosta-5,7,9(10),22-tetraene-3β,20α-diol, (5Z,7E,22E)-9,10-secoergosta-5,7,9(10),22-tetraene-β,20β-diol, (6E,22E)-9,10-secoergosta-5(10),6,8,22-tetraene-3β,20α-diol, (6E,22E)-9,10-secoergosta-5(10),6, 8, 22-tetraene-3β,20β-diol, 9b,10a-ergosta-5,7,22-triene-3β,20α-diol, 9b,10a-ergosta-5,7,22-triene-β,20β-diol, ergosta-5,7,22-triene-3β,20α-diol, or ergosta-5,7,22-triene-β,20β-diol.

23. The method of claim 19, wherein the cytochrome P450scc enzyme system is an in vitro system, comprising:
cytochrome P450scc enzyme;
adrenodoxin;
adrenodoxin reductase; and
NADPH.

24. The method of claim 19, wherein the enzyme system(s) has an in vitro or in vivo mammalian cell comprising an adrenal cell, a gonadal cell, a placental cell, a cell from the gastrointestinal tract, a kidney cell, a brain cell, or a skin cell, a plant cell, an insect cell, a yeast cell, a bacteria or other eukaryotic or prokaryotic cell.

25. The method of claim 24, wherein the enzyme system(s) is a recombinant system in the cell.

26. A hydroxylated cholecalciferol or ergocalciferol derivative or analog compound that is (5Z,7E)-9,10-secocholesta-5,7,10(19)-triene-3β,20-diol, (5Z,7E)-9,10-secocholesta-5,7,10(19)-triene-3β,20,23-triol, (5Z,7E)-9,10-secocholesta-5,7,10(19)-triene-3β,17α,20,23-tetrol, (5Z,7E)-9,10-secochalesta-5,7,10(19)-triene-1α,3β,20,23-tetrol, or (5Z,7E)-9,10-secocholesta-5,7,10(19)-triene-1α,3β,17α,20,23-pentol, (6E)-9,10-secocholesta-5(10),6,8-triene-3β,20α-diol, (6E)-9,10-secocholesta-5(10),6,8-triene-3β,20β-diol, 9β,10α-cholesta-5,7-diene-3β,20α-diol, 9β,10α-cholesta-5,7-diene-3β,20β-diol, cholesta-5,7-diene-3β,20α-diol, cholesta-5,7-diene-β,20β-diol, (5Z,7E,22E)-9,10-secoergosta-5,7,9(10),22-tetraene-3β,20°-diol, (5Z,7E,22E)-9,10-secoergosta-5,7,9(10),22-tetraene-β,20β-diol, (6E,22E)-9,10-secoergosta-5(10),6,8,22-tetraene-3β,20α-diol, (6E,22E)-9,10-secoergosta-5(10),6,8,22-tetraene-3β,20β-diol, 9β,10α-ergosta-5,7,22-triene-3β,20α-diol, 9β,10α-ergosta-5,7,22-triene-β,20β-diol, ergosta-5,7,22-triene-3β,20α-diol, or ergosta-5,7,22-triene-β,20β-diol.
